SEQUENCE LISTING (113 sequences)

SEQ ID NO: 1 - protein, 304 aa, synthetic construct
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 2 - protein, 304 aa, Sus scrofa
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGRITGTVKRKLTSRL

SEQ ID NO: 3 - protein, 304 aa, Papio hamadryas
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVIHSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 4 - protein, 304 aa, synthetic construct
MAHYRNDYKKNDEVEFVRTGYGKDMIKVLHIQRDGKYHSIKEVATSVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 5 - protein, 304 aa, Sus scrofa; identical to SEQ ID NO: 2

SEQ ID NO: 6 - protein, 290 aa, synthetic construct
  REGION 1..290: note = Modified Arthrobacter globiformis Uricase, modified N-terminus, SGD, 2-Cys, C-terminal truncation (SGD V1 C2)
MATAETSTGCKVVLGQNQYGKAEVRLVKVTRCTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 7 - protein, 290 aa, synthetic construct
  REGION 1..290: note = Modified Arthrobacter globiformis Uricase, modified N-terminus, RGD, 2-Cys, C-terminal truncation (RGD V1 C2)
MATAETSTGCKVVLGQNQYGKAEVRLVKVTRCTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 8 - protein, 290 aa, synthetic construct
  REGION 1..290: note = Modified Arthrobacter globiformis Uricase, modified N-terminus, RGD variants, 2-Cys, C-terminal truncation (RGD variants of V1 C2)
  VARIANT 48: note = X is either R or any natural amino acid except C
  VARIANT 49: note = X is either G or any natural amino acid except C
  VARIANT 50: note = X is either D or any natural amino acid except C
MATAETSTGCKVVLGQNQYGKAEVRLVKVTRCTARHEIQDLNVTSQLXXXFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 9 - protein, 301 aa, synthetic construct
  REGION 1..301: note = Genus sequence, with optional N-terminal modification, 4 possible cysteines, R/SGD, optionally with or without C-terminal truncation
  REGION 1..301: note = MISC_FEATURE - From at least one, two, three, or four cysteines are included in the sequence
  VARIANT 2: note = X is either present or absent, and if present is T
  VARIANT 11: note = X is either T or C
  VARIANT 33: note = X is either N or C
  VARIANT 49: note = X is either R or S
  VARIANT 119: note = X is either N or C
  VARIANT 142: note = X is either S or C
  VARIANT 292..301: note = One or more amino acids in the C-terminus are optional
MXATAETSTGXKVVLGQNQYGKAEVRLVKVTRXTARHEIQDLNVTSQLXGDFEAAHTAGDNAHVVATDTQKNTVYAFARDGFATTEEFLLRLGKHFTEGFDWVTGGRWAAQQFFWDRIXDHDHAFSRNKSEVRTAVLEISGXEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGF

SEQ ID NO: 10 - protein, 300 aa, synthetic construct
  REGION 1..300: note = Genus sequence, with modified N-terminus, with 4 possible cysteines, SGD, optionally with or without C-terminal truncation
  REGION 1..300: note = MISC_FEATURE - From at least one, two, three, or four cysteines are included in the sequence
  VARIANT 10: note = X is either T or C
  VARIANT 32: note = X is either N or C
  VARIANT 118: note = X is either N or C
  VARIANT 141: note = X is either S or C
  VARIANT 291..300: note = One or more amino acids in the C-terminus are optional
MATAETSTGXKVVLGQNQYGKAEVRLVKVTRXTARHEIQDLNVTSQLSGDFEAAHTAGDNAHVVATDTQKNTVYAFARDGFATTEEFLLRLGKHFTEGFDWVTGGRWAAQQFFWDRIXDHDHAFSRNKSEVRTAVLEISGXEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGF

SEQ ID NO: 11 - protein, 292 aa, synthetic construct
  REGION 1..292: note = Genus sequence, with truncated N-terminus, 4 possible cysteines, SGD, optionally with or without C-terminal truncation
  REGION 1..292: note = MISC_FEATURE - From at least one, two, three, or four cysteines are included in the sequence
  VARIANT 2: note = X is either T or C
  VARIANT 24: note = X is either N or C
  VARIANT 110: note = X is either N or C
  VARIANT 133: note = X is either S or C
  VARIANT 283..292: note = One or more amino acids in the C-terminus are optional
MXKVVLGQNQYGKAEVRLVKVTRXTARHEIQDLNVTSQLSGDFEAAHTAGDNAHVVATDTQKNTVYAFARDGFATTEEFLLRLGKHFTEGFDWVTGGRWAAQQFFWDRIXDHDHAFSRNKSEVRTAVLEISGXEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGF

SEQ ID NO: 12 - protein, 301 aa, synthetic construct
  REGION 1..301: note = Genus sequence, with optional N-terminal modification, 9 possible cysteines, R/SGD, optionally with or without C-terminal truncation
  VARIANT 1..301: note = From at least one, two, three, or four cysteines are included in the sequence
  VARIANT 2: note = X is either present or absent, and if present is T
  VARIANT 11: note = X is either T or C
  VARIANT 33: note = X is either N or C
  VARIANT 49: note = X is either R or S
  VARIANT 119: note = X is either N or C
  VARIANT 120: note = X is either D or C
  VARIANT 142: note = X is either S or C
  VARIANT 196: note = X is either E or C
  VARIANT 238: note = X is either P or C
  VARIANT 286: note = X is either E or C
  VARIANT 289: note = X is either R or C
  VARIANT 292..301: note = One or more amino acids in the C-terminus are optional
MXATAETSTGXKVVLGQNQYGKAEVRLVKVTRXTARHEIQDLNVTSQLXGDFEAAHTAGDNAHVVATDTQKNTVYAFARDGFATTEEFLLRLGKHFTEGFDWVTGGRWAAQQFFWDRIXXHDHAFSRNKSEVRTAVLEISGXEQAIVAGIEGLTVLKSTGSEFHGFPRDKYTTLQETTDRILATDVSARWRYNTVXVDFDAVYASVRGLLLKAFAETHSLALQQTMYEMGRAVIETHXEIDEIKMSLPNKHHFLVDLQPFGQDNPNEVFYAADRPYGLIEATIQRXGSXADHPIWSNIAGF

SEQ ID NO: 13 - protein, 301 aa, synthetic construct; identical to SEQ ID NO: 12
  REGION 1..301: note = Genus sequence, with optional N-terminal modification, 9 possible cysteines, XGD, optionally with or without C-terminal truncation
  REGION 1..301: note = MISC_FEATURE - From at least one, two, three, or four cysteines are included in the sequence
  VARIANT 2: note = X is either present or absent, and if present is T
  VARIANT 11: note = X is either T or C
  VARIANT 33: note = X is either N or C
  VARIANT 49: note = X is any naturally occurring amino acid except C
  VARIANT 119: note = X is either N or C
  VARIANT 120: note = X is either D or C
  VARIANT 142: note = X is either S or C
  VARIANT 196: note = X is either E or C
  VARIANT 238: note = X is either P or C
  VARIANT 286: note = X is either E or C
  VARIANT 289: note = X is either R or C
  VARIANT 292..301: note = One or more amino acids in the C-terminus are optional

SEQ ID NO: 14 - protein, 292 aa, synthetic construct
  REGION 1..292: note = Genus sequence, with N-terminal truncation, 9 possible cysteines, XGD, optionally with or without C-terminal truncation
  REGION 1..292: note = MISC_FEATURE - From at least one, two, three, or four cysteines are included in the sequence
  VARIANT 2: note = X is either T or C
  VARIANT 24: note = X is either N or C
  VARIANT 40: note = X is any naturally occurring amino acid except C
  VARIANT 110: note = X is either N or C
  VARIANT 111: note = X is either D or C
  VARIANT 133: note = X is either S or C
  VARIANT 187: note = X is either E or C
  VARIANT 229: note = X is either P or C
  VARIANT 277: note = X is either E or C
  VARIANT 280: note = X is either R or C
  VARIANT 283..292: note = One or more amino acids in the C-terminus are optional
MXKVVLGQNQYGKAEVRLVKVTRXTARHEIQDLNVTSQLXGDFEAAHTAGDNAHVVATDTQKNTVYAFARDGFATTEEFLLRLGKHFTEGFDWVTGGRWAAQQFFWDRIXXHDHAFSRNKSEVRTAVLEISGXEQAIVAGIEGLTVLKSTGSEFHGFPRDKYTTLQETTDRILATDVSARWRYNTVXVDFDAVYASVRGLLLKAFAETHSLALQQTMYEMGRAVIETHXEIDEIKMSLPNKHHFLVDLQPFGQDNPNEVFYAADRPYGLIEATIQRXGSXADHPIWSNIAGF

SEQ ID NO: 15 - protein, 301 aa, synthetic construct; identical to SEQ ID NO: 12
  REGION 1..301: note = Genus sequence, with optional N-terminal modification, 9 possible conjugation sites, XGD, optionally with or without C-terminal truncation
  REGION 1..301: note = MISC_FEATURE - From at least one, two, three, or four cysteines are included in the sequence
  VARIANT 2: note = X is either present or absent, and if present is T
  VARIANT 11: note = X is either T or any natural or unnatural amino acid used for site-specific conjugation
  VARIANT 33: note = X is either N or any natural or unnatural amino acid used for site-specific conjugation
  VARIANT 49: note = X is any naturally occurring amino acid except C
  VARIANT 119: note = X is either N or any natural or unnatural amino acid used for site-specific conjugation
  VARIANT 120: note = X is either D or any natural or unnatural amino acid used for site-specific conjugation
  VARIANT 142: note = X is either S or any natural or unnatural amino acid used for site-specific conjugation
  VARIANT 196: note = X is either E or any natural or unnatural amino acid used for site-specific conjugation
  VARIANT 238: note = X is either P or any natural or unnatural amino acid used for site-specific conjugation
  VARIANT 286: note = X is either E or any natural or unnatural amino acid used for site-specific conjugation
  VARIANT 289: note = X is either R or any natural or unnatural amino acid used for site-specific conjugation
  VARIANT 292..301: note = One or more amino acids in the C-terminus are optional

SEQ ID NO: 16 - protein, 292 aa, synthetic construct; identical to SEQ ID NO: 14
  REGION 1..292: note = Genus sequence, with N-terminal truncation, 9 possible conjugation sites, XGD, optionally with or without C-terminal truncation
  REGION 1..292: note = MISC_FEATURE - From at least one, two, three, or four cysteines are included in the sequence
  VARIANT 2: note = X is either T or any natural or unnatural amino acid used for site-specific conjugation
  VARIANT 24: note = X is either N or any natural or unnatural amino acid used for site-specific conjugation
  VARIANT 40: note = X is any naturally occurring amino acid except C
  VARIANT 110: note = X is either N or any natural or unnatural amino acid used for site-specific conjugation
  VARIANT 111: note = X is either D or any natural or unnatural amino acid used for site-specific conjugation
  VARIANT 133: note = X is either S or any natural or unnatural amino acid used for site-specific conjugation
  VARIANT 187: note = X is either E or any natural or unnatural amino acid used for site-specific conjugation
  VARIANT 229: note = X is either P or any natural or unnatural amino acid used for site-specific conjugation
  VARIANT 277: note = X is either E or any natural or unnatural amino acid used for site-specific conjugation
  VARIANT 280: note = X is either R or any natural or unnatural amino acid used for site-specific conjugation
  VARIANT 283..292: note = One or more amino acids in the C-terminus are optional

SEQ ID NO: 17 - protein, 303 aa, synthetic construct
  REGION 1..303: note = Modified Arthrobacter globiformis Uricase C1 construct (T11C mutation, SGD, optional N-terminal His tag and optional short linker (first Uricase residue corresponds to Thr2))
  SITE 1..13: note = Optional N-terminal His tag and optional linker
MGSHHHHHHGARQTATAETSTGCKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 18 - protein, 290 aa, synthetic construct
  REGION 1..290: note = Modified Arthrobacter globiformis Uricase C1 construct (variant 1), with tag eliminated, deletion of Thr2 (to avoid partial N-term Met cleavage) and Cys at position 11
MATAETSTGCKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 19 - protein, 287 aa, synthetic construct
  REGION 1..287: note = Modified Arthrobacter globiformis Uricase C1 construct (variant 2 - N-term truncation) with tag eliminated, deletion of Thr2-Ala5 and Cys at position 11, expect complete retention of N-term Met
METSTGCKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 20 - protein, 283 aa, synthetic construct
  REGION 1..283: note = Modified Arthrobacter globiformis Uricase C1 construct (variant 3 - N-term truncation) with tag eliminated, deletion of Thr2-Thr9, Cys at position 11, expect processing of N-term met
MGCKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 21 - protein, 303 aa, synthetic construct
  REGION 1..303: note = Modified Arthrobacter globiformis Uricase with SGD, and PEGylation available sites at T11C, N33C, S142C, optional N-terminal His tag and optional short linker
  SITE 1..13: note = Optional N-terminal His tag and optional linker
MGSHHHHHHGARQTATAETSTGCKVVLGQNQYGKAEVRLVKVTRCTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGCEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 22 - protein, 303 aa, synthetic construct
  REGION 1..303: note = Modified Arthrobacter globiformis Uricase, NH2-terminal truncated, SGD, PEGylation available sites at T11C and N33C 2-Cys (SGD His C2) with optional N-terminal His tag and optional short linker
  SITE 1..13: note = Optional N-terminal His tag and optional linker
MGSHHHHHHGARQTATAETSTGCKVVLGQNQYGKAEVRLVKVTRCTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 23 - protein, 290 aa, synthetic construct
  REGION 1..290: note = Modified Arthrobacter globiformis Uricase (C-term truncation with SGD)
MATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 24 - protein, 289 aa, synthetic construct
  REGION 1..289: note = Modified Arthrobacter globiformis Uricase (processed form - Met cleaved at N-term., SGD, and C-term truncation)
ATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 25 - protein, 303 aa, synthetic construct
  REGION 1..303: note = Modified Arthrobacter globiformis Uricase (contains optional N-terminal His tag and optional short linker, contains SGD instead of RGD) (C-term truncation with his tag and SGD)
  SITE 1..13: note = Optional N-terminal His tag and optional linker
MGSHHHHHHGARQTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 26 - protein, 303 aa, synthetic construct
  REGION 1..303: note = Modified Arthrobacter globiformis Uricase (contains optional N-terminal His tag and optional short linker) (C-term truncation with his tag)
  SITE 1..13: note = Optional N-terminal His tag and optional linker
MGSHHHHHHGARQTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 27 - protein, 291 aa, synthetic construct
  REGION 1..291: note = Modified Arthrobacter globiformis Uricase (0 cysteines) (truncated the C-terminal 11 amino acids to eliminate the Cys) (C-term truncation)
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 28 - protein, 291 aa, synthetic construct
  REGION 1..291: note = Modified Arthrobacter globiformis Uricase (0 cysteines) (RGD variants, truncated the C-terminal 11 amino acids to eliminate the Cys) (C-term truncation)
  VARIANT 49: note = X is either R or any natural amino acid except C
  VARIANT 50: note = X is either G or any natural amino acid except C
  VARIANT 51: note = X is either D or any natural amino acid except C
MTATAETSTGTKVVLGQNQYGKAEVRLVKVTRNTARHEIQDLNVTSQLXXXFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 29 - protein, 301 aa, synthetic construct
  REGION 1..301: note = Modified Arthrobacter globiformis Uricase (0 cysteines) (truncated the C-terminal aa to eliminate the cysteine)
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGF

SEQ ID NO: 30 - protein, 301 aa, synthetic construct
  REGION 1..301: note = Modified Arthrobacter globiformis Uricase (0 cysteines) (RGD variants, truncated the C-terminal aa to eliminate the cysteine)
  VARIANT 49: note = X is either R or any natural amino acid except C
  VARIANT 50: note = X is either G or any natural amino acid except C
  VARIANT 51: note = X is either D or any natural amino acid except C
MTATAETSTGTKVVLGQNQYGKAEVRLVKVTRNTARHEIQDLNVTSQLXXXFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGF

SEQ ID NO: 31 - protein, 302 aa, synthetic construct
  REGION 1..302: note = Modified Arthrobacter globiformis Uricase (RGD variants) (contains the C-terminal 11 amino acids)
  VARIANT 49: note = X is either R or any natural amino acid
  VARIANT 50: note = X is either G or any natural amino acid
  VARIANT 51: note = X is either D or any natural amino acid
MTATAETSTGTKVVLGQNQYGKAEVRLVKVTRNTARHEIQDLNVTSQLXXXFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 32 - protein, 302 aa, Arthrobacter globiformis
MTATAETSTGTKVVLGQNQYGKAEVRLVKVTRNTARHEIQDLNVTSQLRGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRADHPIWSNIAGFC

SEQ ID NO: 33 - protein, 293 aa, Deinococcus geothermalis
MTQTQQNQQPKVKARLGANNYGKAEVNLMKVKRDSERHEIRELQVRVALIGDFAAAHEQGDNTDLLATDTVRNTIYGLAKEGFQASPEAFGKELISHFVTTGPKVTGGFMEFTEYLWERIQVGGEGHNHAFVRQMPQRTGRVESEDGKTFKITSGLQNLYVLKTTESGWANYLLNERFTTLPETHERLMASFVTAKWEYNEDQVDYDDVWPRVYRQLQETFTDHYSPSLQRTLFLMGQAVLTRCPEMSRIWLQMPNKHHLQYNLERFGLDNNLEIFHVDPEPYGLMEAWVERA

SEQ ID NO: 34 - protein, 298 aa, Deinococcus radiodurans
MMTGTQQPGTQPKVKVRLGENNYGKAEVQLMKIKRGTPRHELREAKVRVAMYGDFGAAHSEGDNTDLVATDTVRNTVYGLAKEGFESSIEEFGKELLTHFVKVGPRVTGGFAEFTEHLWERVQTPAQPQGHDHAFVRQMPKRTARVETQDGRRFTVTSGIEELYVLKTTESGWENYLLDERFTTLPETHDRVMATFVTAKWEYAVESCDYDAVWERVYRQIQHTFTDHYSPSLQRTLYLMGEAVLSVCPEISRIWFQMPNKHHLVYNLGRFGLENNNEILHVDPEPYGLMEAWVERAE

SEQ ID NO: 35 - protein, 287 aa, Granulicella tundricola
MAELTDAKFEIVANRYGKSKVRLLKVTRAEGRSDVHEWTVQVLLRGDFETAHTVGDNSKIVTTDTMKNTVYSLARWSSATTMEEFAEELIEHLLRRNEQVSSVRVHIEAALWKRLTVDGKEHPDTFMRGSNEVQTATVEQARAGEKKFIAGFANLQLLKTANSAFSGFQRDELTTLPETRDRVFGTAVDAKWTYSGPVEFAAMRKAAREVMLKVFADHMSESVQHTLYAMADAALEAVAEITEIELAMPNKHCLLVDLSKFGQDNPNQIFVPTDEPHGYIEARVRRK

SEQ ID NO: 36 - protein, 287 aa, Candidatus Solibacter usitatus
MERFASGWKQNYYGKGDVIVYRLNRDGVVPQGCCPVFGANVKMLLYGDAFWPTYTTGDNTNLVATDSMKNFIQRETCNFTGYDLESYCDFLARKFMATYPHTAGIQLSARQAPYSGVAEGKVAFAPSGPDVATACVELRRNGEALESVEASSGIHGFRLLRLGGSAFQGFLRDQYTTLPDIHNRPLHMWLDLEWHYIAPEAALTGGEVTAQVRRLVHEGFHSFESGSIQQVIYQLGTKMLADIPTISEVRLEANNRTWDTIVEQGDRLGVYTDARPPYGCLGLTLRR

SEQ ID NO: 37 - protein, 280 aa, Terriglobus saanensis
MAKLIDSRYGKARVRVMKLDRSQPQHQLLEWTVRVLLEGDFETAHTVGDNSNILPTDTMKNTVYSRAKESKAETPEEFAIELAEFLLGRNPQVHTVEVKIETAMWKRLVVDGKPHGSSFMRGSDELGTVLHHATRETKTMVCGVENMVILKSQNSSFEGYIQDDLTTLKPTADRLFATAMTADWDYTDGGSAFAARREAIREAMLKAFAEHDSKSVQQTLYAMAEAAMAAVPAVNRVHMVMPNKHCLLVDLKHFGQENNNEIFVPTEDPHGYIEATVVRE

SEQ ID NO: 38 - protein, 326 aa, Kyrpidia tusciae
MIMTGTMTSGTDQRTMYYGKGDVWVYRSYAKPLRGLGQIPESAFAGRPNVIFGMNVQMAVEGEAFLPSFTEGDNSMVVATDSMKNFILRQAGAFEGATAEGFLEFVAGKFLEKYAHVSGVRLFGRQIPFDELPVPEQEGFRPGELVFRYSMNEYPTAFVAVRRGPEGPVVVEHAGGVAGLKLIKIKGSSFYGYIHDEYTTLPEAQDRPLFIYLYIKWKYEHPEDFRAEHPERYVAAEQVRDIAHTVFHELTSPSIQNLIYHIGRRVLTRFPQLLEVSFEANNRTWETVLEEVEDLAGKRAEAKVYTEPRPPYGFQGFTVTRKDLEE

SEQ ID NO: 39 - protein, 304 aa, synthetic construct
  REGION 1..304: note = Consensus Uricase sequence from alignment
MTATAETSTGTKIVLGQNQYGKAEVRVVKITRDGDTHHIKDLNVSVALSGDMDAVHLSGDNANVLPTDTQKNTVYAFAKEHGIGSAEQFGIRLARHFVTSQEPIHGARIRIEEYAWERIETSHDHSFVRKGQETRTAQITYDGDWEVVSGLKDLTVLNSTGSEFWGYVKDKYTTLPETYDRILATDVSARWRYNWTDDQPMPDWDKSYEQVRKHLLEAFAETYSLSLQQTLYQMGSRVLEARPEIDEIRFSLPNKHHFLVDLEPFGLDNDNEVYFAADRPYGLIEATVLRDGAEPRIPVDMTNL

SEQ ID NO: 40 - protein, 299 aa, synthetic construct
  REGION 1..299: note = Synthetic Pig-KS-DeltaN polypeptide
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 41 - protein, 298 aa, synthetic construct
  REGION 1..298: note = Synthetic Pig-KS-DeltaN without starting Met polypeptide
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 42 - protein, 296 aa, synthetic construct
  REGION 1..296: note = Synthetic PBC-DeltaNC polypeptide
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLS

SEQ ID NO: 43 - protein, 295 aa, synthetic construct
  REGION 1..295: note = Synthetic PBC-DeltaNC without starting Met polypeptide
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLSLSRVPEIEDMEISLPNIHYFNIDMSKMGLINKEEVLLPLDNPYGKITGTVKRKLS

SEQ ID NO: 44 - protein, 304 aa, Papio hamadryas
MADYHNNYKKNDELEFVRTGYGKDMVKVLHIQRDGKYHSIKEVATSVQLTLSSKKDYLHGDNSDIIPTDTIKNTVHVLAKFKGIKSIEAFGVNICEYFLSSFNHVIRAQVYVEEIPWKRLEKNGVKHVHAFIHTPTGTHFCEVEQLRSGPPVITSGIKDLKVLKTTQSGFEGFIKDQFTTLPEVKDRCFATQVYCKWRYHQCRDVDFEATWGTIRDLVLEKFAGPYDKGEYSPSVQKTLYDIQVLSLSRVPEIEDMEISLPNIHYFNIDMSKMGLINKEEVLLPLDNPYGKITGTVKRKLSSRL

SEQ ID NO: 45 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 46 - protein, 290 aa, synthetic construct; identical to SEQ ID NO: 6

SEQ ID NO: 47 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 48 - protein, 20 aa, synthetic construct
ASPAAPAPASPAAPAPSAPA

SEQ ID NO: 49 - DNA, 60 bp, synthetic construct
  source 1..60: mol_type = other DNA
gcctctccagctgcacctgctccagcaagccctgctgcaccagctccgtctgctcctgct

SEQ ID NO: 50 - protein, 20 aa, synthetic construct
AAPASPAPAAPSAPAPAAPS

SEQ ID NO: 51 - DNA, 60 bp, synthetic construct
  source 1..60: mol_type = other DNA
gctgctccggcttccccggctccggctgctccgtccgctccggctccggctgctccgtcc

SEQ ID NO: 52 - protein, 20 aa, synthetic construct
APSSPSPSAPSSPSPASPSS

SEQ ID NO: 53 - DNA, 60 bp, synthetic construct
  source 1..60: mol_type = other DNA
gctccgtcctccccgtccccgtccgctccgtcctccccgtccccggcttccccgtcctcc

SEQ ID NO: 54 - protein, 20 aa, synthetic construct
SSPSAPSPSSPASPSPSSPA

SEQ ID NO: 55 - DNA, 60 bp, synthetic construct
  source 1..60: mol_type = other DNA
tcctccccgtccgctccgtccccgtcctccccggcttccccgtccccgtcctccccggct

SEQ ID NO: 56 - protein, 24 aa, synthetic construct
AASPAAPSAPPAAASPAAPSAPPA

SEQ ID NO: 57 - DNA, 72 bp, synthetic construct
  source 1..72: mol_type = other DNA
gccgcttctccagcagctccttctgctccaccagcagctgcaagccctgctgcaccaagcgcacctcctgct

SEQ ID NO: 58 - protein, 20 aa, synthetic construct
ASAAAPAAASAAASAPSAAA

SEQ ID NO: 59 - DNA, 60 bp, synthetic construct
  source 1..60: mol_type = other DNA
gcctctgctgcagcacctgcagcagcaagcgcagctgcatctgctccatctgcagctgct

SEQ ID NO: 60 - protein, 200 aa, synthetic construct
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPA

SEQ ID NO: 61 - protein, 400 aa, synthetic construct
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPA

SEQ ID NO: 62 - protein, 600 aa, synthetic construct
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPA

SEQ ID NO: 63 - protein, 507 aa, synthetic construct
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

SEQ ID NO: 64 - protein, 707 aa, synthetic construct
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

SEQ ID NO: 65 - protein, 907 aa, synthetic construct
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

SEQ ID NO: 66 - protein, 733 aa, synthetic construct
MTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSAGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEAAHHHHHH

SEQ ID NO: 67 - protein, 507 aa, synthetic construct
MASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 68 - protein, 707 aa, synthetic construct
MASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 69 - protein, 907 aa, synthetic construct
MASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 70 - protein, 744 aa, synthetic construct
MGSSHHHHHHSAGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEAASTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 71 - protein, 6 aa, synthetic construct
HQHQHQ

SEQ ID NO: 72 - protein, 12 aa, synthetic construct
HNHNHNHNHNHN

SEQ ID NO: 73 - protein, 19 aa, synthetic construct
KDHLIHNVHKEEHAHAHNK

SEQ ID NO: 74 - protein, 425 aa, synthetic construct
AGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEA

SEQ ID NO: 75 - protein, 1119 aa, synthetic construct
MGSSHHHHHHSSGASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

SEQ ID NO: 76 - protein, 1319 aa, synthetic construct
MGSSHHHHHHSSGASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

SEQ ID NO: 77 - protein, 519 aa, synthetic construct
MGSSHHHHHHSSGASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 78 - protein, 919 aa, synthetic construct
MGSSHHHHHHSSGASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 79 - protein, 735 aa, synthetic construct
MSAGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEAASTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 80 - protein, 719 aa, synthetic construct
MGSSHHHHHHSSGASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSHHHHHH

SEQ ID NO: 81 - DNA, 60 bp, synthetic construct
  source 1..60: mol_type = other DNA
gctagccccgcggcgccggcgccggcttctcctgctgcgccggcgccgagcgcgccggcg

SEQ ID NO: 82 - DNA, 60 bp, synthetic construct
  source 1..60: mol_type = other DNA
gcgagcccggcggcgccggcgccggcttctcctgctgcgccggcgccgagcgcgccggcg

SEQ ID NO: 83 - DNA, 60 bp, synthetic construct
  source 1..60: mol_type = other DNA
gcttctcctgccgcgccggcgccggctagtcctgcagctcctgctcctagcgcgccggcg

SEQ ID NO: 84 - DNA, 60 bp, synthetic construct
  source 1..60: mol_type = other DNA
gctagtcctgcagcgccggcgccggctagccctgcggctcctgctcccagcgcgccggcg

SEQ ID NO: 85 - DNA, 60 bp, synthetic construct
  source 1..60: mol_type = other DNA
gctagccctgcggcgccggcgccggcctctcccgctgctcctgctccaagcgcgccggcg

SEQ ID NO: 86 - DNA, 60 bp, synthetic construct
  source 1..60: mol_type = other DNA
gcctctcccgctgcgccggcgccggccagtcccgccgctcctgctccgtctgcgccggcg

SEQ ID NO: 87 - DNA, 60 bp, synthetic construct
  source 1..60: mol_type = other DNA
gcttctcccgccgcgccggcgccggccagccctgctgctcctgctccgagcgcgccggcg

SEQ ID NO: 88 - DNA, 60 bp, synthetic construct
  source 1..60: mol_type = other DNA
gccagtcccgcagcgccggcgccggcatctcccgcggctcctgccccgagcgcgccggcg

SEQ ID NO: 89 - DNA, 60 bp, synthetic construct
  source 1..60: mol_type = other DNA
gcatctcccgcggccccggcgccggcttctcccgcggctcctgcaccgagcgcgccggcg

SEQ ID NO: 90 - DNA, 60 bp, synthetic construct
  source 1..60: mol_type = other DNA
gcgtctcctgcggctcctgcgccggcttctccagctgctcctgcgccttctgcgccggcg

SEQ ID NO: 91 - DNA, 60 bp, synthetic construct
  source 1..60: mol_type = other DNA
gctagtcccgcggctccggcgccggcttctccagccgctcctgcgcctagtgcgccggcg

SEQ ID NO: 92 - DNA, 60 bp, synthetic construct
  source 1..60: mol_type = other DNA
gctagccccgcggcgccggcgccggcaagtccagctgctcccgcaccttctgcgccggcg

SEQ ID NO: 93 - DNA, 60 bp, synthetic construct
  source 1..60: mol_type = other DNA
gcctctccagctgcgccggcgccggcaagccctgctgctcctgcgcctagcgcgccggcg

SEQ ID NO: 94 - DNA, 60 bp, synthetic construct
  source 1..60: mol_type = other DNA
gccagccctgctgccccggcgccggctagtccagccgctcccgcacctagtgcgccggcg

SEQ ID NO: 95 - DNA, 60 bp, synthetic construct
  source 1..60: mol_type = other DNA
gccagtccagccgcgccggcgccggcgtctcctgcggcccctgcgccctctgcgccggcg

SEQ ID NO: 96 - DNA, 60 bp, synthetic construct
  source 1..60: mol_type = other DNA
gccagccccgctgctccggcgccggctagtcccgcggcacctgcgcccagtgcgccggcg

SEQ ID NO: 97 - DNA, 60 bp, synthetic construct
  source 1..60: mol_type = other DNA
gcatctccagcagcgccggcgccggcgagtcctgcggctcccgcacctagcgctccggcg

SEQ ID NO: 98 - DNA, 60 bp, synthetic construct
  source 1..60: mol_type = other DNA
gcttctccagcggctccggcgccggcctctccagcggcccctgcgcccagcgcgccggcg

SEQ ID NO: 99 - DNA, 60 bp, synthetic construct
  source 1..60: mol_type = other DNA
gcaagtccagcggccccggcgccggcctctcctgcggctcccgccccttctgcgccggcg

SEQ ID NO: 100 - DNA, 60 bp, synthetic construct
  source 1..60: mol_type = other DNA
gcaagtcccgcggcacccgcgccggcttctccagcggcacctgcgccaagcgcgccggcg

SEQ ID NO: 101 - DNA, 60 bp, synthetic construct
  source 1..60: mol_type = other DNA
gcaagccctgctgcaccggcgccggcttctccggctgctcctgcgccatctgcgccggcg

SEQ ID NO: 102 - DNA, 60 bp, synthetic construct
  source 1..60: mol_type = other DNA
gcaagccccgctgccccggcgccggccagtccagcggcacccgctccatctgctccggcg

SEQ ID NO: 103 - DNA, 60 bp, synthetic construct
  source 1..60: mol_type = other DNA
gcttctcccgcggcaccagcgccggcttctcctgcggctcccgctccatctgccccggcg

SEQ ID NO: 104 - DNA, 60 bp, synthetic construct
  source 1..60: mol_type = other DNA
gcttctcctgcggcccccgcgccggcttctccggccgctcctgcgccaagtgcgccggcg

SEQ ID NO: 105 - DNA, 60 bp, synthetic construct
  source 1..60: mol_type = other DNA
gcgtctcccgcggcaccggctccggcttctccagcggcgcctgcgccgtctgctccggcg

SEQ ID NO: 106 - DNA, 60 bp, synthetic construct
  source 1..60: mol_type = other DNA
gctagtccagcggcaccggcgccggctagtccggctgctcccgctccatctgcaccggcg

SEQ ID NO: 107 - DNA, 60 bp, synthetic construct
  source 1..60: mol_type = other DNA
gcttctccggctgccccggcgccggcatctccagcggcgcccgcacctagcgccccggcg

SEQ ID NO: 108 - DNA, 60 bp, synthetic construct
  source 1..60: mol_type = other DNA
gcttctccggccgccccggcgccggctagcccagcggcgcctgcgccgagtgcgccggcg

SEQ ID NO: 109 - DNA, 60 bp, synthetic construct
  source 1..60: mol_type = other DNA
gcgagtcctgcggccccagcgccggcttctccggcagctcctgcgccaagcgctccggcg

SEQ ID NO: 110 - DNA, 60 bp, synthetic construct
  source 1..60: mol_type = other DNA
gctagtcctgcggctcccgcgccggctagtccagcggcgcccgcccctagtgcgccggcg

SEQ ID NO: 111 - DNA, 60 bp, synthetic construct
  source 1..60: mol_type = other DNA
gcgagccctgctgctcccgcgccggctagcccggctgctcctgcgccgtctgccccggcg

SEQ ID NO: 112 - protein, 1153 aa, synthetic construct
MSAGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEAASTYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLAGSPTPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPTAEAAGGTAEAA

SEQ ID NO: 113 - protein, 1107 aa, synthetic construct
MASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPATYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRLGSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAHHHHHH

(19) United States
(12) Patent Application Publication
(10) Pub. No.: US 2026/0201346 A1
(43) Pub. Date: Jul. 16, 2026

(54) URICASE CONJUGATES AND METHODS OF USE THEREOF

(71) Applicant: Insmed Incorporated, Bridgewater, NJ (US)

(72) Inventors: Patrick WIENCEK, Bridgewater, NJ (US); Claire GODBERSEN-PALMER, Bridgewater, NJ (US); Hongki SONG, Bridgewater, NJ (US); Karl GRISWOLD, Bridgewater, NJ (US); Nicholas ROSENTHAL, Bridgewater, NJ (US)

(21) Appl. No.: 19/136,669

(22) PCT Filed: Dec. 8, 2023
(86) PCT No.: PCT/US2023/083137

§ 371 Date: Jun. 6, 2025

Related U.S. Application Data
(60) Provisional application No. 63/386,660, filed on Dec. 8, 2022.

(51) Int. Cl. C12N 9/06; A61K 9/00; A61K 38/00

(52) U.S. Cl. CPC C12N 9/0048; A61K 9/0019; C12Y 107/03003; A61K 38/00; C07K 2319/00

(57) ABSTRACT

The present invention provides novel uricase conjugates comprising a first domain and a second domain. The first domain comprises a uricase polypeptide and the second domain is an unstructured, random coil polypeptide domain comprising at least about 100 amino acids. Methods for using the uricase conjugates of the invention, for example, in the treatment of tumor lysis syndrome and/or gout, are also provided.

### CROSS REFERENCE TO RELATED APPLICATION

This application claims priority from U.S. Provisional Application Ser. No. 63/386,660, filed Dec. 8, 2022, the disclosure of which is incorporated by reference herein in its entirety.

### REFERENCE TO AN ELECTRONIC SEQUENCE LISTING

The contents of the electronic sequence listing (INMD_186_01WO_SeqList_ST26.xml; Size: 184,960 bytes; and Date of Creation: Dec. 7, 2023) are herein incorporated by reference in its entirety.

### BACKGROUND OF THE INVENTION

In humans, uric acid is produced following breakdown of purines. Accumulation of uric acid in the blood (hyperuricemia) is manifested in diseases, such as gout and tumor lysis syndrome.

Gout is a common and complex form of arthritis characterized by sudden, severe attacks of pain, swelling, redness and tenderness in one or more joints. Gout is caused by accumulation of uric acid crystals in the joints, leading to inflammation of intense pain. Uric acid crystals are formed when high levels of uric acid are present in the blood.

Tumor lysis syndrome is a complication from the treatment of cancer, e.g., lymphomas, leukemias, including non-Hodgkin lymphoma, acute myeloid leukemia, and acute lymphoblastic leukemia. Tumor lysis syndrome occurs as large numbers of tumor cells are lysed, releasing their contents into the bloodstream. Tumor lysis syndrome is characterized by high blood uric acid (hyperuricemia), as well as high blood potassium (hyperkalemia), high blood phosphate (hyperphosphatemia), low blood calcium (hypocalcemia), and higher than normal levels of blood urea nitrogen (BUN). The metabolic abnormalities seen in tumor lysis syndrome can ultimately result in serious complications, such as acute uric acid nephropathy, acute kidney failure, seizures, cardiac arrhythmias, and death.

Uricase, sometimes referred to as uric oxidase, is the enzyme that catalyzes the oxidation of uric acid to a more soluble product, allantoin, a purine metabolite which is more readily excreted. Because humans do not produce enzymatically active uricase, due to several mutations in the gene for uricase acquired during the evolution of higher primates, exogenously administered uricase provides a therapy for diseases manifesting hyperuricemia, such as gout and tumor lysis syndrome.

The present invention addresses the need for effective treatments of gout, tumor lysis syndrome, and other diseases associated with hyperuricemia, by providing novel uricase conjugates and methods for using them in the treatment of the aforementioned diseases.

### SUMMARY OF THE INVENTION

In one aspect of the invention, a uricase conjugate comprising a first domain and a second domains is provided. The first domain comprises a uricase polypeptide, or an amino acid variant thereof, and the second domain is a first random coil polypeptide domain comprising at least about 100 amino acids.

In one embodiment of the uricase conjugate provided herein, the uricase conjugate is a fusion protein of the first domain and the second domain. In a further embodiment, the first domain (uricase polypeptide) is C-terminal to the second domain (the first random coil polypeptide).

In one embodiment of a uricase fusion protein, an amino acid linker, for example, an amino acid linker comprising from about 2 to about 5 amino acids, is present between the first domain and the second domain. In a further embodiment, the linker is Gly-Ser.

In another embodiment, the uricase conjugate is a uricase fusion protein comprising three domains: a uricase polypeptide domain (first domain); a first random coil polypeptide domain (second domain) and a second random coil polypeptide domain (third domain). The two random coil polypeptide domains can be the same or different. For each of the (first and second) random coil polypeptide domains, at least about 100 amino acids are present within the respective domain.

A random coil polypeptide domain, in embodiments described herein, comprises from about 100 amino acids to about 600 amino acids, e.g., from about 11 amino acids to about 300 amino acids.

The random coil polypeptide domain, in one embodiment, comprises a Pro-Ala-Ser (PAS) polypeptide. In a further embodiment, the PAS polypeptide has the amino acid sequence set forth in SEQ ID NO: 60. In another embodiment, the PAS polypeptide has the amino acid sequence set forth in SEQ ID NO: 61. In yet another embodiment, the PAS polypeptide has the amino acid sequence set forth in SEQ ID NO: 62.

In yet even another embodiment, the PAS polypeptide has an amino acid sequence set forth in SEQ ID NO: 48, SEQ ID NO: 50, SEQ ID NO: 52, SEQ ID NO: 54, SEQ ID NO: 56 or SEQ ID NO: 58. In a further embodiment, the PAS polypeptide has the amino acid sequence set forth in SEQ ID NO: 48. In one embodiment, the amino acid sequence of SEQ ID NO: 48 is encoded by a nucleotide sequence selected from SEQ ID NOs: 81-111.

The random coil polypeptide domain, in one embodiment, comprises an extended recombinant (XTEN) polypeptide. In a further embodiment, the XTEN polypeptide has the amino acid sequence set forth in SEQ ID NO: 74.

The uricase domain of the uricase conjugate described herein, in one embodiment, comprises the amino acid sequence set forth in SEQ ID NO: 1.

In another embodiment, the uricase domain of the uricase conjugate comprises the amino acid sequence set forth in SEQ ID NO: 2.

In another embodiment, the uricase domain of the uricase conjugate comprises the amino acid sequence set forth in SEQ ID NO: 3.

In even another embodiment, the uricase domain of the uricase conjugate comprises the amino acid sequence set forth in SEQ ID NO: 4.

In yet even another embodiment, the uricase domain of the uricase conjugate comprises the amino acid sequence set forth in SEQ ID NO: 5.

The uricase domain of the uricase conjugate described herein, in another embodiment, comprises an amino acid sequence selected from SEQ ID NO: 6-39.

The uricase domain of the uricase conjugate described herein, in another embodiment, comprises an amino acid sequence selected from SEQ ID NO: 40-44.

The uricase domain of the uricase conjugate described herein, in yet another embodiment, comprises the amino acid sequence set forth in SEQ ID NO: 40.

The uricase domain of the uricase conjugate described herein, in yet another embodiment, comprises the amino acid sequence set forth in SEQ ID NO: 41.

The uricase domain of the uricase conjugate described herein, in yet even another embodiment, comprises the amino acid sequence set forth in SEQ ID NO: 45.

The uricase domain of the uricase conjugate described herein, in even yet another embodiment, comprises the amino acid sequence set forth in SEQ ID NO: 46.

In another embodiment, the uricase domain of the uricase conjugate comprises the amino acid sequence set forth in SEQ ID NO: 47.

In yet even another embodiment, the uricase domain is an amino acid variant of SEQ ID NO: 40. In a further embodiment, the amino acid variant comprises from about 10 to about 20 amino acid substitutions. In even a further embodiment, the amino acid variant comprises from about 10 to about 16 or from about 10 to about 15 amino acid substitutions.

In yet even another embodiment, the uricase domain is an amino acid variant of SEQ ID NO: 41. In a further embodiment, the amino acid variant comprises from about 10 to about 20 amino acid substitutions. In even a further embodiment, the amino acid variant comprises from about 10 to about 16 or from about 10 to about 15 amino acid substitutions.

In one embodiment of a uricase conjugate described herein, the uricase conjugate is a fusion protein. In a further embodiment, the fusion protein, upon expression, forms a homotetratmer.

In another embodiment of a uricase fusion protein described herein, the uricase fusion protein has an amino acid sequence selected from SEQ ID NO:63, 64, 65, 66, 67, 68, 69, 70, 75, 76, 77, 78, 79, 80, 112, and 113.

In another aspect of the invention, a nucleic acid molecule is provided that encodes a uricase conjugate, or a domain thereof, as described herein. The nucleic acid molecule, in one embodiment, is present in a vector to allow for in vitro expression of the uricase conjugate.

In yet another aspect of the invention, a method of treatment is provided comprising administering to a subject in need of treatment, an effective amount of one of the uricase conjugates described herein, or a pharmaceutical composition comprising the same. The method of treatment in one embodiment, is a method of treating hyperuricemia. As such, in one embodiment provided herein, uric acid levels in the subject, e.g., in the blood or plasma of the subject, are reduced when administered an effective amount of a uricase conjugate of the invention, or a pharmaceutical composition comprising the same.

In another embodiment, the method of treatment is a method of treating gout. In a further embodiment, the gout is refractory gout. In a further embodiment, uric acid levels in the subject, e.g., in the blood or plasma of the subject, are reduced when administered an effective amount of a uricase conjugate of the invention, or a pharmaceutical composition comprising the same.

In even another embodiment, the method of treatment is a method of treating tumor lysis syndrome. In a further embodiment, uric acid levels in the subject, e.g., in the blood or plasma of the subject, are reduced when administered an effective amount of a uricase conjugate of the invention, or a pharmaceutical composition comprising the same.

In one embodiment of a method of treating a subject provided herein, the subject is an adult human subject.

In one embodiment of a method provided herein, the method comprises parenteral administration to the subject. In a further embodiment, the parenteral administration is intravenous administration. In yet another embodiment, the method comprises subcutaneous administration to the subject.

In another aspect, the present disclosure provides a method of recombinantly producing a uricase conjugate (e.g., a recombinant uricase fusion protein) disclosed herein. The method includes (i) culturing a host cell comprising a nucleic acid vector comprising a nucleic acid sequence encoding the uricase conjugate (e.g., the recombinant uricase fusion protein) disclosed herein, wherein the nucleic acid sequence is operatively linked to a heterologous promoter under conditions to allow for expression of the nucleic acid sequence encoding the uricase conjugate (e.g., the recombinant uricase fusion protein) and recombinant production of the uricase conjugate (e.g., the uricase fusion protein) by the host cell; and (ii) isolating the recombinantly produced uricase conjugate (e.g., recombinant uricase fusion protein).

### BRIEF DESCRIPTION OF THE DRAWINGS

FIG. 1 is a schematic showing various configurations of uricase fusion proteins of the invention.

FIG. 2A is an image of an SDS-PAGE gel showing bands representing monomeric NPAS20 h-CPB41 uricase-CPAS20h, NPAS20 h-CPB41 uricase-CPAS30h, and pegloticase in lanes 1, 2, and 3, respectively, detected by Coomassie Brilliant Blue staining.

FIG. 2B is an image of an SDS-PAGE gel showing bands representing monomeric CPB40 uricase-CPAS20 h and NPAS20 h-CPB41 uricase-CPAS20 h in lanes 1 and 2, respectively, detected by Coomassie Brilliant Blue staining.

FIG. 3A is a graph showing plasma concentrations of the endogenous uricase as well as uric acid in untreated control Wistar rats (n=3) at the corresponding pre-dose timepoint (at time 0) and at various corresponding post-dose timepoints up to 96 h in pharmacokinetic (PK) study 1. Each data point is presented as mean±SD.

FIG. 3B is a graph showing plasma concentrations of pegloticase and uric acid pre-dose (at time 0) and at various timepoints up to 96 h post-dose in Wistar rats (n=3) intravenously administrated with a single dose of 1 mg/kg body weight pegloticase in PK study 1. Each data point is presented as mean±SD.

FIG. 3C is a graph showing plasma concentrations of CPB40 uricase-CPAS10 h and uric acid pre-dose (at time 0) and at various timepoints up to 96 h post-dose in Wistar rats (n=3) intravenously administrated with a single dose of 1.51 mg/kg body weight CPB40 uricase-CPAS10 h in PK study 1. Each data point is presented as mean±SD.

FIG. 3D is a graph showing plasma concentrations of CPB40 uricase-CPAS20 h and uric acid pre-dose (at time 0) and at various timepoints up to 96 h post-dose in Wistar rats (n=3) intravenously administrated with a single dose of 1.99 mg/kg body weight CPB40 uricase-CPAS20 h in PK study 1. Each data point is presented as mean±SD.

FIG. 3E is a graph showing plasma concentrations of CPB40 uricase-CPAS30 h and uric acid pre-dose (at time 0) and at various timepoints up to 96 h post-dose in Wistar rats (n=3) intravenously administrated with a single dose of 2.47 mg/kg body weight CPB40 uricase-CPAS30 h in PK study 1. Each data point is presented as mean±SD.

FIG. 3F is a graph showing plasma concentrations of CPB40 uricase-CXTENh and uric acid pre-dose (at time 0) and at various timepoints up to 96 h post-dose in Wistar rats (n=3) intravenously administrated with a single dose of 2.16 mg/kg body weight CPB40 uricase-CXTENh in PK study 1. Each data point is presented as mean±SD.

FIG. 4A is a graph showing plasma concentrations of the endogenous uricase as well as uric acid in untreated control Wistar rats (n=3) at the corresponding pre-dose timepoint (at time 0) and at various corresponding post-dose timepoints up to 168 h in PK study 2. Each data point is presented as mean±SD.

FIG. 4B is a graph showing plasma concentrations of pegloticase and uric acid pre-dose (at time 0) and at various timepoints up to 168 h post-dose in Wistar rats (n=3) intravenously administrated with a single dose of 1 mg/kg body weight pegloticase in PK study 2. Each data point is presented as mean±SD.

FIG. 4C is a graph showing plasma concentrations of NPAS20 h-CPB41 uricase-CPAS20 h and uric acid pre-dose (at time 0) and at various timepoints up to 168 h post-dose in Wistar rats (n=3) intravenously administrated with a single dose of 2.99 mg/kg body weight NPAS20 h-CPB41 uricase-CPAS20 h in PK study 2. Each data point is presented as mean±SD.

FIG. 4D is a graph showing plasma concentrations of NPAS20 h-CPB41 uricase-CPAS30 h and uric acid pre-dose (at time 0) and at various timepoints up to 168 h post-dose in Wistar rats (n=3) intravenously administrated with a single dose of 3.47 mg/kg body weight NPAS20 h-CPB41 uricase-CPAS30 h in PK study 2. Each data point is presented as mean±SD.

FIG. 5A is a graph showing plasma concentrations of the endogenous uricase as well as uric acid in untreated control Wistar rats (n=3) at the corresponding pre-dose timepoint (at time 0) and at various corresponding post-dose timepoints up to 240 h in PK study 3. Each data point is presented as mean±SD.

FIG. 5B is a graph showing plasma concentrations of CPB40 uricase-CPAS20 h and uric acid pre-dose (at time 0) and at various timepoints up to 240 h post-dose in Wistar rats (n =3) intravenously administrated with a single dose of 1.99 mg/kg body weight CPB40 uricase-CPAS20 h in PK study 3. Each data point is presented as mean±SD.

FIG. 5C is a graph showing plasma concentrations of NPAS20 h-CPB41 uricase-CPAS20 h and uric acid pre-dose (at time 0) and at various timepoints up to 240 h post-dose in Wistar rats (n=3) intravenously administrated with a single dose of 2.99 mg/kg body weight NPAS20 h-CPB41 uricase-CPAS20 h in PK study 3. Each data point is presented as mean±SD.

### DETAILED DESCRIPTION OF THE INVENTION

The present invention, in one aspect, relates to a novel conjugate molecule comprising two domains: (i) a uricase enzyme conjugated to a (ii) random coil polypeptide. Conjugation, as described in preferred embodiments, is accomplished at the DNA level via operably linking a uricase DNA sequence to a DNA sequence encoding a random coil polypeptide, followed by recombinant expression of the continuous DNA sequence. The linking of the two domains at the DNA level obviates the need for in vitro coupling or modification steps to achieve conjugate synthesis, which is needed, for example, for the coupling of polyethylene glycol (PEG) to uricase in the case of the approved uricase molecule, KRYSTEXXA® (pegloticase).

In preferred embodiments described herein, the random coil polypeptide comprises the three amino acids, proline (Pro), alanine (Ala) and serine (Ser). In a further embodiment, the random coil polypeptide consists of the three amino acids, proline (Pro), alanine (Ala) and serine (Ser). Where the amino acid residues in a polypeptide are all Pro, Ala and Ser, or substantially all the amino acids are Pro, Ala and Ser in a polypeptide, such a polypeptide is referred to herein as a “PAS polypeptide”.

In another embodiment, the random coil polypeptide is an extended recombinant (XTEN) polypeptide. The XTEN polypeptide, in one embodiment, is one of the polypeptides disclosed in U.S. Patent Application Publication No. 2015/0037359, the content of which is incorporated by reference in its entirety for all purposes. The XTEN polypeptide, in one embodiment, is at least about 800 amino acids in length consisting of six hydrophilic chemically stable amino acids Ala, Asp, Gly, Pro, Ser, and Thr, in a nonrepetitive manner. In one embodiment, the XTEN polypeptide is 864 residues long. In another embodiment, the XTEN polypeptide is a fragment of the 864 aa XTEN polypeptide. In one embodiment, the XTEN polypeptide comprises the amino acid sequence set forth in SEQ ID NO:74. XTEN polypeptides may be conjugated to the uricase enzymes described herein via chemical conjugation or produced as a fusion protein with the uricase enzyme.

In embodiments where the uricase conjugate is produced via recombinant expression of a single DNA sequence, the uricase conjugate is referred to herein as a uricase “fusion protein”. Specifically, a “fusion protein” refers to a protein composed of a plurality of polypeptide components, that while typically unjoined in their native state, are joined by their respective N-terminus and C-terminus through a peptide linkage to form a single continuous polypeptide. Uricase fusion proteins may be a combination of two, three or four or more different proteins. Uricase fusion proteins can also include fusions with heterologous and homologous leader sequences, with or without N-terminal methionine residues; as well as fusion proteins that include additional sequences for purification of the fusion protein (e.g., a polyhistidine tag).

The uricase conjugate, in one embodiment, comprises at least two domains. The first domain of the at least two domains comprises a uricase enzyme and the second domain of the at least two domains comprises a random coil polypeptide comprising at least about 100 amino acid residues, e.g., a PAS polypeptide or an XTEN polypeptide. Without wishing to be bound by theory, the random coil conformation mediates an increased in vivo and/or in vitro stability of the uricase enzyme. Moreover, without wishing to be bound by theory, because the random coil polypeptide domain is thought to not adopt a stable structure or function by itself, the biological activity mediated by the uricase to which it is conjugated is essentially preserved.

In another aspect of the invention, a nucleic acid molecule is provided. The nucleic acid molecule, in one embodiment, encodes the uricase conjugate (i.e., the uricase fusion protein). Also provided, in some embodiments, are nucleic acid vectors and cells comprising the nucleic acid molecule encoding the uricase fusion protein. Other aspects of the invention relate to compositions comprising the conjugates of the invention as well as specific uses of the compositions, e.g., to treat gout and tumor lysis syndrome.

As used herein, the term “domain” relates to any region/part of an amino acid sequence that is capable of autonomously adopting a specific structure and/or function. In the context of the present invention, accordingly, a “domain” may represent a functional domain or a structural domain. As described herein, the proteins of the present invention comprise at least one uricase domain and at least one domain/part forming random coil conformation (e.g., the PAS polypeptide domain). The uricase conjugates of the invention also may include more than two domains. For example, as provided herein, because uricase exists as a homotetramer, in one embodiment, a polypeptide conjugate of the present invention comprises four uricase domains and four random coil domains. Moreover, the fusion proteins of the present invention may comprise e.g., an additional linker structure between the herein defined two domains/parts or another domain/part like, e.g. a protease sensitive cleavage site, an affinity tag such as the polyhistidine tag or the Strep-tag, a signal peptide, retention peptide, a targeting peptide like a membrane translocation peptide or additional effector domains like antibody fragments for tumor targeting associated with an anti-tumor toxin or an enzyme for prodrug-activation etc. In another embodiment of a uricase fusion protein, a uricase domain is a uricase monomer and the fusion protein further comprises a random coil domain C-terminal to the uricase domain and a random coil domain N-terminal to the uricase domain. In a further embodiment, the random coil domains comprise PAS polypeptides.

Uricase (EC 1.7.3.3) exists in microorganisms (such as Bacillus fastidiosus, Candida mycoderma and Aspergillus flavus), plants (such as beans and chickpeas), and animals (such as pigs, cows, dogs, and baboons) (Suzuki Ket al., J. Biosci. Bioeng., 2004, 98:153-158). The enzyme initiates a series of reactions that convert uric acid (UA) to the more soluble and easily excreted product, allantoin. Briefly, uricase catalyzes the reaction of UA with O₂ and water (H₂O) to form 5-hydroxy-isourate (HIU) and the release of hydrogen peroxide (H₂O₂). HIU then undergoes non-enzymatic hydrolysis to 2-oxo-4-hydroxy-4-carboxy-5-ureidoimidazoline (OHCU) which then decarboxylates spontaneously to form allantoin. Nyborg et al. (2016). PLoS One 11 (12): e0167935. Doi: 10.1371/journal.pone.0167935; Ramazzina et al. (2006). Nature Chemical Biology 2 (2), pp. 144-148.

Humans lack a functional uricase due to three mutations that result in complete silencing of the human uricase gene. Although it was hypothesized that the lack of uricase was beneficial from an evolutionary perspective, in modern humans, high uric acid may have negative consequences due to urate deposition, and an increase in gout. As such, the uricase conjugates described herein are provided as therapeutics in one aspect of the invention, to treat patients having elevated UA levels.

The active uricase is a tetrameric protein with four identical subunits (i.e., uricase is a homotetramer), each having molecular weight of about 34 kD and consisting of 301-304 amino acids. Uricase has maximum enzymatic activity at pH 8.0 (Bayol A et al., Biophys. Chem. 1995, 54:229-235). Among all origins, uricase has the highest activity from Aspergillus flavus, which is up to 27 IU/mg: the second highest from Bacillus fastidiosus with 13 IU/mg (Huang S H et al., Eur. J. Biochem., 2004, 271:517-523). Additionally, bean-origined uricases have activities of merely 2-6 IU/mg. As for recombinantly expressed mammal uricases, the activity of pig uricase can reach 5 IU/mg, and papio uricase only 1 IU/mg (Michael Het al., 2006, U.S. Pat. No. 7,056,713, incorporated by reference herein in its entirety); while human uricase has no activity.

The present invention is not limited by the source or the amino acid sequence of the uricase enzyme domain of the conjugate molecule. For example, the uricase provided in the conjugate of the invention is a wild type uricase enzyme or an amino acid variant thereof. For example, the uricase, in one embodiment, is a dog, pig, cow, goat or baboon uricase, or is derived from a dog, pig, cow, goat or baboon uricase. In one embodiment, the uricase provided in a conjugate of the invention, is an amino acid variant of a dog, pig, cow, goat or baboon uricase. Uricase amino acid variants, as used herein, differ in amino acid sequence from the counterpart wild type uricase enzyme, but still retain uricase activity.

In one embodiment, the uricase is an amino acid variant of a known uricase enzyme. In a further embodiment, the uricase amino acid variant has an amino acid sequence with at least about 75%, for example, at least about 75%, at least about 80%, at least about 85%, at least about 90%, at least about 95%, at least about 96%, at least about 97%, at least about 98%, or at least about 99%, including all values and subranges that lie therebetween, identity to the amino acid sequence of the known uricase enzyme. In even a further embodiment, the known uricase enzyme has the amino acid sequence set forth in SEQ ID NO:40 or SEQ ID NO:41. Each of the uricase enzymes of SEQ ID NO:40 and SEQ ID NO:41 is a chimeric pig-baboon uricase comprising amino acids (aa) 8-266 of porcine uricase (SEQ ID NO:2) and aa 267-304 of baboon uricase (SEQ ID NO:3). Hence, the uricase enzymes of SEQ ID NO:40 and SEQ ID NO: 41 are also referred to as “CPB40 uricase” and “CPB41 uricase,” respectively, in the present application. Except for lacking the N-terminal methionine, the amino acid sequence of SEQ ID NO:41 is otherwise identical to the amino acid sequence of SEQ ID NO:40. KRYSTEXXA® (pegloticase) is a hyper-PEGylated homotetrameric protein and each monomer is a single polypeptide chain comprised of the chimeric pig-baboon uricase having the amino acid sequence of SEQ ID NO:40 or 41.

In one embodiment, the uricase is a uricase amino acid variant. The uricase amino acid variant comprises from about 10 to about 20 amino acid substitutions, for example, about 10, about 11, about 12, about 13, about 14, about 15, about 16, about 17, about 18, about 19 or about 20 amino acid substitutions. In another embodiment, the uricase amino acid variant comprises from about 10 to about 20 amino acid substitutions, for example, about 10 to about 18, about 10 to about 16, about 10 to about 14, about 10 to about 13, or about 10 to about 12 amino acid substitutions.

The uricase amino acid variant, in one embodiment, is an amino acid variant of a pig-baboon chimeric uricase. In a further embodiment, the uricase amino acid variant is a variant of the uricase polypeptide sequence set forth in SEQ ID NO:40. In even a further embodiment, the uricase amino acid variant of SEQ ID NO:40 comprises from about 10 to about 20 amino acid substitutions, for example, about 10 to about 18, about 10 to about 16, about 10 to about 14, about 10 to about 13, or about 10 to about 12 amino acid substitutions. In another embodiment, the uricase amino acid variant is a variant of the uricase polypeptide sequence set forth in SEQ ID NO:41. In a further embodiment, the uricase amino acid variant of SEQ ID NO: 41 comprises from about 10 to about 20 amino acid substitutions, for example, about 10 to about 18, about 10 to about 16, about 10 to about 14, about 10 to about 13, or about 10 to about 12 amino acid substitutions.

The uricase conjugate provided herein, in preferred embodiments, is a recombinant uricase fusion protein. “Recombinant protein”, as used herein, refers to any artificially produced protein and is distinguished from naturally produced proteins (i.e., proteins are produced in tissues of an animal that possesses only the natural gene for the specific protein of interest). As described herein, the recombinant uricase fusion protein comprises a uricase domain bonded via a peptide bond (directly or through an amino acid linker) to one or more random coil polypeptide domains.

The uricase domain, in one embodiment, comprises a recombinant molecule which includes segments of porcine and baboon liver uricase proteins. In another embodiment, the uricase domain comprises a recombinant modified baboon uricase. In one embodiment, the uricase domain comprises a chimeric pig-baboon uricase (PBC uricase, SEQ ID NO:1)) which includes amino acids (aa) 1-225 of porcine uricase (SEQ ID NO:2) and aa 226-304 of baboon uricase (SEQ ID NO:3). In another embodiment, the uricase is a chimeric pig-baboon uricase which includes aa 1-288 of porcine uricase and aa 289-304 of baboon uricase (PKS uricase, SEQ ID NO: 4), or a variant of one of the foregoing, or a truncated version of one of the foregoing.

The uricase in one embodiment, is a chimeric pig-baboon uricase.

The uricase in one embodiment, is a pig uricase having an amino acid sequence set forth in SEQ ID NO: 5.

In one embodiment, the uricase is a humanized uricase, e.g., the uricase can comprise amino acids from non-active human uricase substituted into the amino acid sequence of a non-human mammal uricase, to retain the original activity as well as to improve the homology with human uricase, thus reducing immunogenicity in human body. Such humanized uricase enzymes are disclosed in U.S. Pat. No. 8,586,535, the disclosure of which is incorporated by reference in its entirety.

In one embodiment, the uricase is an Aspergillus flavus uricase or is derived from Aspergillus flavus uricase.

In one embodiment, the uricase is an Arthrobacter globiformis uricase (NCBI Accession no. DOVWQ1), Deinococcus geothermalis uricase (NCBI Accession no. WP_011525965), Deinococcus radiodurans uricase (NCBI Accession no. WP_010887803), Granulicella tundricola uricase (NCBI Reference Sequence: WP_013581210.1), Solibacter usitatus uricase (NCBI Accession no. WP_011682147), Terriglobus saanensis uricase (NCBI Accession no. WP_013569963), a Kyrpidia tusciae uricase (NCBI Accession no. ADG06709), or an amino acid variant of one of the foregoing.

Uricase genes and proteins have been identified in several mammalian species, for example, pig, baboon, rat, rabbit, mouse, and rhesus monkey. The sequences of various uricase proteins are described herein by reference to their public data base accession numbers, as follows: gi|50403728|sp|P25689; gi|20513634|dbj|BAB91555.1; gi|176610|AAA35395.1; gi|20513654|dbj|BAB91557.1; gi|47523606|ref|NP_999435.1; gi|6678509|ref|NP_033500.1; gi|57463|emb|CAA31490.1; gi|20127395|ref|NP_446220.1; gi|137107|sp|P11645; gi|51458661|ref|XP_497688.1; gi|207619|gb|AAA42318.1; gi|26340770|dbj|BAC34047.1; and gi|57459|emb|CAA30378.1. Each of these sequences and their annotations in the public databases accessible through the National Center for Biotechnology Information (NCBI) is incorporated by reference in its entirety for all purposes.

In an embodiment of the invention, the uricase domain of the uricase conjugate comprises a mammalian uricase, or an amino acid variant thereof. In a further embodiment, the mammalian uricase comprises the amino acid sequence of porcine, bovine, ovine or baboon liver uricase. In an embodiment of the invention, the uricase is a chimeric uricase of two or more mammalian uricases. In a further embodiment, the mammalian uricases of the chimeric uricase are selected from porcine, bovine, ovine, or baboon liver uricase.

In an embodiment, the uricase domain of the uricase conjugate comprises a fungal or microbial uricase. In a further embodiment, the fungal or microbial uricase is an Aspergillus flavus, Arthrobacter globiformis or Candida utilis uricase. In yet another embodiment, the uricase domain of the uricase conjugate comprises an invertebrate uricase. In a further embodiment, the invertebrate uricase is a Drosophila melanogaster or a Drosophila pseudoobscura uricase.

In another embodiment, the uricase domain of the uricase conjugate comprises a Candida utilis uricase.

In even another embodiment, the uricase domain of the uricase conjugate comprises a plant uricase. In a further embodiment, the plant uricase is Glycine max uricase of root nodules.

In even another embodiment, the uricase portion of the conjugate has an amino acid sequence selected from one of SEQ ID NOS: 6-39, or an amino acid variant thereof (Table 1).

TABLE 1

| | SEQ |
|---|---|
| | ID |
| Uricase Enzyme | NO |
| Modified Arthrobacter globiformis uricase | 6 |
| Modified Arthrobacter globiformis uricase | 7 |
| Modified Arthrobacter globiformis uricase | 8 |
| Uricase genus sequence | 9 |
| Uricase genus sequence | 10 |
| Uricase genus sequence | 11 |
| Uricase genus sequence | 12 |
| Uricase genus sequence | 13 |
| Uricase genus sequence | 14 |
| Uricase genus sequence | 15 |
| Uricase genus sequence | 16 |
| Modified Arthrobacter globiformis uricase | 17 |
| Modified Arthrobacter globiformis uricase | 18 |
| Modified Arthrobacter globiformis uricase | 19 |
| Modified Arthrobacter globiformis uricase | 20 |
| Modified Arthrobacter globiformis uricase | 21 |
| Modified Arthrobacter globiformis uricase | 22 |
| Modified Arthrobacter globiformis uricase | 23 |
| Modified Arthrobacter globiformis uricase | 24 |
| Modified Arthrobacter globiformis uricase | 25 |
| Modified Arthrobacter globiformis uricase | 26 |
| Modified Arthrobacter globiformis uricase | 27 |
| Modified Arthrobacter globiformis uricase | 28 |
| Modified Arthrobacter globiformis uricase | 29 |
| Modified Arthrobacter globiformis uricase | 30 |
| Modified Arthrobacter globiformis uricase | 31 |
| Arthrobacter globiformis uricase | 32 |
| Deinococcus geothermalis uricase | 33 |
| Deinococcus radiodurans uricase | 34 |
| Granulicella tundricola uricase | 35 |
| Acidic Bacteria Solibacter usitatus uricase | 36 |
| Terriglobus saanensis Acidobacterium uricase | 37 |
| Kyrpidia tusciae uricase | 38 |
| Consensus uricase | 39 |

In yet even another embodiment, the uricase is a synthetic uricase generated from the consensus uricase amino acid sequence derived from the alignment of 50 uricase sequences that shared the greatest identity to Arthrobacter globiformis uricase (SEQ ID NO: 39).

In one embodiment, the uricase enzyme can be a wild type uricase enzyme or an engineered variant thereof. For example, in one embodiment, the uricase enzyme is a recombinant mammalian uricase.

In one embodiment, the uricase has an amino acid sequence disclosed in U.S. Pat. No. 10,731,139, the content of which is incorporated by reference in its entirety for all purposes.

Methods for mutating amino acids are well-known in the art, and such methods can be used to mutate one or more amino acids of a wild type uricase enzyme to produce a uricase amino acid variant. For example, an Arg-Gly-Asp (RGD), a tripeptide reported to mediate cell adhesion through integrin binding, can be mutated to a Ser-Gly-Asp (SGD) sequence such that the uricase domain does include an RGD sequence.

In one embodiment, the uricase portion of the conjugate is a truncated uricase. A “truncated uricase”, as used herein, refers to a uricase molecule having a shortened primary amino acid sequence compared to a known uricase enzyme's amino acid sequence (e.g., a wild type enzyme). The truncation, in one embodiment, is a truncation at or around the N- and/or C-terminus of the uricase. In one embodiment, the uricase is truncated at the N-terminus. In a further embodiment, the N-terminal truncation begins at position 1, 2, 3, 4, 5 or 6. In one embodiment, the amino terminal truncation begins at position 2, thereby leaving out the amino terminal methionine (Met). The amino terminal Met may be removed, in one embodiment, by post-translational modification. In another embodiment, the amino terminal Met is removed after the uricase is produced as one of the fusion proteins described herein. In a further embodiment, the Met is removed by endogenous bacterial aminopeptidase.

In an embodiment of a truncated uricase, the uricase is truncated by 4-13 amino acids at its N-terminus. In another embodiment, the uricase is truncated by 4-13 amino acids at its C-terminus. In an embodiment of a truncated uricase, the uricase is truncated by 4-13 amino acids at both its C-terminus and N-terminus. In another embodiment, the uricase is truncated by 6 amino acids at its N-terminus. In even another embodiment, the uricase for use in one of the conjugates, e.g., fusion proteins described herein, is truncated by 6 amino acids at its C-terminus. In an embodiment of the invention, the uricase for use in one of the conjugates, e.g., fusion proteins described herein, is truncated by 6 amino acids at both its carboxy and amino termini.

In one embodiment of the uricase conjugate described herein, the uricase domain of the conjugate comprises the amino acid sequence set forth in a sequence selected from SEQ ID NO: 40-44. Like the uricase enzymes of SEQ ID NO:40 and SEQ ID NO:41, the uricase enzymes of SEQ ID NO:42 and SEQ ID NO:43 are each a chimeric pig-baboon uricase comprising aa 8-220 of porcine uricase (SEQ ID NO:2) and aa 221-301 of baboon uricase (SEQ ID NO:3). Except for lacking the N-terminal methionine, the amino acid sequence of SEQ ID NO:43 is otherwise identical to the amino acid sequence of SEQ ID NO:42.

In a further embodiment, the uricase protein comprises the amino acid sequence set forth in SEQ ID NO: 1. In another embodiment, the uricase protein comprises the amino acid sequence set forth in SEQ ID NO: 40 or 41.

In one embodiment, the uricase protein comprises the amino acid sequence set forth in SEQ ID NO: 7. In yet another embodiment, the uricase protein comprises the amino acid sequence set forth in SEQ ID NO: 40, 41, 42, 43 or 44. In one embodiment of the invention, the uricase comprises the amino acid sequence of SEQ ID NO. 41. In another embodiment of the invention, the uricase comprises the amino acid sequence of SEQ ID NO. 43.

In yet another embodiment, the uricase domain comprises one of the uricase polypeptides disclosed in PCT Publication No. 2016/187026, the content of which are incorporated by reference in its entirety.

In one embodiment, the uricase domain comprises rasburicase, marketed under the trade name Elitek®, and having the amino acid sequence set forth in SEQ ID NO: 45.

In another embodiment, the uricase domain comprises the uricase having the amino acid sequence set forth in SEQ ID NO: 46, and also referred to as HZN-003.

In yet another embodiment, the uricase domain comprises the uricase having the amino acid sequence set forth in SEQ ID NO: 47, and also referred to as SEL-212.

Shortcomings of prior art uricase therapeutics include (i) lack of solubility, (ii) immunogenicity, and (iii) fast clearance from circulation via kidney filtration, the latter of which strongly hampers efficacy both in animal studies and in human therapy. To this end, the present invention provides uricase conjugates comprising a conformationally disordered (random coil) polypeptide domain comprising the amino acid residues Pro, Ala and Ser (PAS). PAS sequences are hydrophilic, uncharged biological polymers with biophysical properties similar to polyethylene glycol (PEG), whose chemical conjugation to drugs is an established method for plasma half-life extension and to reduce immunogenicity (e.g., KRYSTEXXA® (pegloticase)). In contrast to PEG modification, PAS polypeptides offer fusion to a uricase on the genetic level, i.e., they are genetically encodable polypeptides and permit host (e.g., Escherichia coli, others) production of fully active uricase fusion proteins and thus obviating in vitro coupling or modification steps.

As mentioned above, the uricase conjugates provided herein comprise at least two domains. The uricase domain is described above. The second domain is referred to herein in some embodiments, as a PAS domain, and comprises an amino acid sequence comprising at least about 100 amino acid residues that form a random coil conformation. The at least about 100 amino acid residues forming the random coil comprise the amino acids proline (Pro), alanine (Ala) and serine (Ser). In a PAS domain, all or substantially all of the amino acids are Pro, Ala and Ser. Without wishing to be bound by theory, the random coil conformation mediates an increased in vivo and/or in vitro stability of the uricase enzyme. Details regarding various types of PAS polypeptides and nucleic acids encoding the same, for use in the present invention, can be found in PCT Publication No. WO 2008/155134, the content of which is incorporated by reference in its entirety for all purposes.

As used herein, the term “random coil” or “random coil polypeptide domain” relates to a conformation of a polymeric molecule, including amino acid polymers, in which the individual monomeric elements that form the polymeric structure are essentially randomly oriented towards the adjacent monomeric elements while still being chemically bound to said adjacent monomeric elements. In particular, a polypeptide or amino acid polymer adopting/having/forming “random coil” conformation substantially lacks a defined secondary and tertiary structure. The nature of polypeptide random coils and their methods of experimental identification are known to the person skilled in the art.

The random coil is formed under physiological conditions. For example, in one embodiment, the physiological conditions are the parameters that are typically valid for higher forms of life, and in particular mammals, most preferably humans. As such, physiological conditions can be the conditions that are normally found in the body fluids of mammals. The physiological conditions may relate to the corresponding parameters found in the healthy body as well as the parameters as found in sick mammals or human patients. For example, a sick mammal or human patient may have a higher, yet physiological temperature condition when said mammal or said human suffers from fever.

Several buffers in experimental settings (e.g., for use in the determination of protein structures, in particular in circular dichroism (CD) measurements and other methods for determining the structural properties of a protein/amino acid stretch), solvents and/or excipients for pharmaceutical compositions, are considered to represent physiological solutions and/or physiological conditions in vitro. Examples of such buffers are, e.g., phosphate-buffered saline, Tris buffers, acetate buffers, citrate buffers or similar buffers. Generally, the pH of a buffer representing physiological solution conditions is in a range from 6.5 to 8.5, e.g., in a range from 7.0 to 8.0, e.g., in a range from 7.2 to 7.7 and the osmolarity may lie in a range from 10 to 1000 mmol/kg H₂O, more particularly in a range from 50 to 500 mmol/kg H₂O, e.g., in a range from 200 to 350 mmol/kg H₂O.

Methods for determining whether an ammo acid polymer forms/adopts random coil conformation are known in the art. Such methods include CD spectroscopy, which represents a light absorption spectroscopy method in which the difference in absorbance of right- and left-circularly polarized light by a substance is measured. The secondary structure of a protein can be determined by CD spectroscopy using far-ultraviolet spectra with a wavelength between approximately 190 and 250 nm. At these wavelengths, the different secondary structures commonly found in polypeptides can be analyzed, since α-helix, parallel and anti-parallel β-sheet and random coil conformations each give rise to a characteristic shape and magnitude of the CD spectrum. Accordingly. by using CD spectrometry the skilled artisan is readily capable of determining whether an amino acid polymer forms/adopts random coil conformation at physiological conditions. Other established biophysical methods include nuclear magnetic resonance (NMR) spectroscopy, absorption spectrometry, infrared and Raman spectrometry, measurement of the hydrodynamic volume via size exclusion chromatography, analytical ultracentrifugation or dynamic/static light scattering as well as measurements of the frictional coefficient or intrinsic viscosity.

In one embodiment, a random coil domain comprises at least about 100 amino acid residues, at least about 150 amino acid residues, at least about 200 amino acid residues, at least about 250 amino acid residues, at least about 300 amino acid residues, at least about 350 amino acid residues, or at least about 400 amino acid residues. In another embodiment, the random coil domain comprises maximally about 1000 amino acid residues, maximally about 900 amino acid residues, maximally about 800 amino acid residues, maximally about 700 amino acid residues, or maximally about 600 amino acid residues. In one embodiment, the random coil domain comprises maximally about 500 amino acid residues or maximally about 450 amino acid residues.

In one embodiment, the random coil polypeptide domain comprises about 100 to about 3000 amino acid residues. In a further embodiment, the random coil domain comprises about 100 to 1000 amino acid residues. In one embodiment, the random coil polypeptide domain comprises an amino acid sequence whereby Pro residues represent about 4% to about 40% of the random coil polypeptide domain. In a further embodiment, alanine and serine residues comprise the remaining about 60% to about 96% of the random coil polypeptide. In some embodiments, the random coil polypeptide domain comprises further amino acids differing from Ala, Ser and Pro, i.e., as minor constituents. The term “minor constituent” as used in this context means that maximally 10% of the amino acids in a random coil polypeptide domain are different from alanine, serine and proline, e.g., maximally 8% of the amino acids in a random coil polypeptide domain, e.g., maximally 6% of the amino acids in a random coil polypeptide domain, e.g., maximally 5% of the amino acids in a random coil polypeptide domain, maximally 4% of the amino acids in a random coil polypeptide domain, maximally 3% of the amino acids in a random coil polypeptide domain, maximally 2% of the amino acids in a random coil polypeptide domain, maximally 1% of the amino acids in a random coil polypeptide domain are different from Ala, Ser and Pro. In one embodiment of a random coil polypeptide domain, the polypeptide comprises amino acids other than Ala, Ser and Pro, and the other amino acids are selected from the group consisting of Arg, Asn, Asp, Cys, Gln, Glu, Gly, His, Ile, Leu, Lys, Met, Phe, Thr, Trp, Tyr, and Val. In another embodiment, the other amino acids include one or more non-natural amino acids.

In another embodiment, the random coil polypeptide is an extended recombinant (XTEN) polypeptide. The XTEN polypeptide, in one embodiment, is one of the polypeptides disclosed in U.S. Patent Application Publication No. 2015/0037359, the content of which is incorporated by reference in its entirety for all purposes. The XTEN polypeptide, in one embodiment, is at least about 800 amino acids in length consisting of six hydrophilic chemically stable amino acids Ala, Asp, Gly, Pro, Ser, and Thr, in a nonrepetitive manner. In one embodiment, the XTEN polypeptide is 864 residues long. In another embodiment, the XTEN polypeptide is a fragment of the 864 aa XTEN polypeptide. XTEN polypeptides may be conjugated to the uricase enzymes described herein via chemical conjugation or produced as a fusion protein with the uricase enzyme.

In another embodiment, the random coil polypeptide domain comprises a plurality of “amino acid repeats”, i.e., the same amino acid sequence occurring two or more times in the domain, wherein the “amino acid repeats” consist of Ala, Ser, and Pro residues (depicted herein as “PAS”, or as “APS”). In a further embodiment, no more than 6 consecutive amino acid residues are identical in the random coil polypeptide domain and the Pro residues constitute more than about 4% and less than about 40% of the amino acids of the random coil polypeptide domain. Non-limiting examples of “amino acid repeats” consisting of Ala, Ser and Pro residues are provided herein; see, e.g., SEQ ID NO: 48, SEQ ID NO: 50, SEQ ID NO: 52, SEQ ID NO: 54, SEQ ID NO: 56 and SEQ ID NO: 58 (Table 2A). Fragments and/or multimers of these sequences are employed in some embodiments. A “fragment” comprises at least 3 amino acids and comprises at least one Ala, one Ser and/or one Pro.

TABLE 2A
Exemplary PAS amino acid sequences for use
in a random coil polypeptide domain, and
nucleotide sequences encoding the same.

| SEQ | |
|---|---|
| ID | |
| NO: | Sequence |
| 48 | ASPAAPAPASPAAPAPSAPA |
| 49 | GCCTCTCCAGCTGCACCTGCTCCAGCAAGCCCTGC |
| | TGCACCAGCTCCGTCTGCTCCTGCT |
| 50 | AAPASPAPAAPSAPAPAAPS |
| 51 | GCTGCTCCGGCTTCCCCGGCTCCGGCTGCTCCGTC |
| | CGCTCCGGCTCCGGCTGCTCCGTCC |
| 52 | APSSPSPSAPSSPSPASPSS |
| 53 | GCTCCGTCCTCCCCGTCCCCGTCCGCTCCGTCCTC |
| | CCCGTCCCCGGCTTCCCCGTCC-TCC |
| 54 | SSPSAPSPSSPASPSPSSPA |
| 55 | TCCTCCCCGTCCGCTCCGTCCCCGTCCTCCCCGGC |
| | TTCCCCGTCCCCGTCCTCCCCGGCT |
| 56 | AASPAAPSAPPAAASPAAPSAPPA |
| 57 | GCCGCTTCTCCAGCAGCTCCTTCTGCTCCACCAGC |
| | AGCTGCAAGCCCTGCTGCACCAAGCGCACCTCCTG |
| | CT |
| 58 | ASAAAPAAASAAASAPSAAA |
| 59 | GCCTCTGCTGCAGCACCTGCAGCAGCAAGCGCAGC |
| | TGCATCTGCTCCATCTGCAGCTGCT |

The aforementioned repeat sequences may be encoded by nucleic acid molecules having the sequences set forth in SEQ ID NO: 49, SEQ ID NO: 51, SEQ ID NO: 53, SEQ ID NO: 55, SEQ ID NO: 57 and/or SEQ ID NO: 59 (Table 2A).

In one embodiment, a nucleotide sequence is provided that encodes the amino acid repeat of SEQ ID NO:48. In a further embodiment, the nucleotide sequence is selected from one of the nucleotide sequences set forth in Table 2B, i.e., one of SEQ ID NOs: 81-111.

TABLE 2B
Exemplary nucleotide sequences encoding
the PAS amino acid repeat of SEQ ID NO: 48

| SEQ | |
|---|---|
| ID | |
| NO: | Sequence |
| 81 | GCTAGCCCCGCGGCGCCGGCGCCGGCTTCTCCTGC |
| | TGCGCCGGCGCCGAGCGCG |
| | CCGGCG |
| 82 | GCGAGCCCGGCGGCGCCGGCGCCGGCTTCTCCTGC |
| | TGCGCCGGCGCCGAGCGCGCCGGCG |
| 83 | GCTTCTCCTGCCGCGCCGGCGCCGGCTAGTCCTGC |
| | AGCTCCTGCTCCTAGCGCGCCGGCG |
| 84 | GCTAGTCCTGCAGCGCCGGCGCCGGCTAGCCCTGC |
| | GGCTCCTGCTCCCAGCGCGCCGGCG |
| 85 | GCTAGCCCTGCGGCGCCGGCGCCGGCCTCTCCCGC |
| | TGCTCCTGCTCCAAGCGCGCCGGCG |
| 86 | GCCTCTCCCGCTGCGCCGGCGCCGGCCAGTCCCGC |
| | CGCTCCTGCTCCGTCTGCGCCGGCG |
| 87 | GCTTCTCCCGCCGCGCCGGCGCCGGCCAGCCCTGC |
| | TGCTCCTGCTCCGAGCGCGCCGGCG |
| 88 | GCCAGTCCCGCAGCGCCGGCGCCGGCATCTCCCGC |
| | GGCTCCTGCCCCGAGCGCGCCGGCG |
| 89 | GCATCTCCCGCGGCCCCGGCGCCGGCTTCTCCCGC |
| | GGCTCCTGCACCGAGCGCGCCGGCG |
| 90 | GCGTCTCCTGCGGCTCCTGCGCCGGCTTCTCCAGC |
| | TGCTCCTGCGCCTTCTGCGCCGGCG |
| 91 | GCTAGTCCCGCGGCTCCGGCGCCGGCTTCTCCAGC |
| | CGCTCCTGCGCCTAGTGCGCCGGCG |
| 92 | GCTAGCCCCGCGGCGCCGGCGCCGGCAAGTCCAGC |
| | TGCTCCCGCACCTTCTGCGCCGGCG |
| 93 | GCCTCTCCAGCTGCGCCGGCGCCGGCAAGCCCTGC |
| | TGCTCCTGCGCCTAGCGCGCCGGCG |
| 94 | GCCAGCCCTGCTGCCCCGGCGCCGGCTAGTCCAGC |
| | CGCTCCCGCACCTAGTGCGCCGGCG |
| 95 | GCCAGTCCAGCCGCGCCGGCGCCGGCGTCTCCTGC |
| | GGCCCCTGCGCCCTCTGCGCCGGCG |
| 96 | GCCAGCCCCGCTGCTCCGGCGCCGGCTAGTCCCGC |
| | GGCACCTGCGCCCAGTGCGCCGGCG |
| 97 | GCATCTCCAGCAGCGCCGGCGCCGGCGAGTCCTGC |
| | GGCTCCCGCACCTAGCGCTCCGGCG |
| 98 | GCTTCTCCAGCGGCTCCGGCGCCGGCCTCTCCAGC |
| | GGCCCCTGCGCCCAGCGCGCCGGCG |
| 99 | GCAAGTCCAGCGGCCCCGGCGCCGGCCTCTCCTGC |
| | GGCTCCCGCCCCTTCTGCGCCGGCG |
| 100 | GCAAGTCCCGCGGCACCCGCGCCGGCTTCTCCAGC |
| | GGCACCTGCGCCAAGCGCGCCGGCG |
| 101 | GCAAGCCCTGCTGCACCGGCGCCGGCTTCTCCGGC |
| | TGCTCCTGCGCCATCTGCGCCGGCG |
| 102 | GCAAGCCCCGCTGCCCCGGCGCCGGCCAGTCCAGC |
| | GGCACCCGCTCCATCTGCTCCGGCG |
| 103 | GCTTCTCCCGCGGCACCAGCGCCGGCTTCTCCTGC |
| | GGCTCCCGCTCCATCTGCCCCGGCG |
| 104 | GCTTCTCCTGCGGCCCCCGCGCCGGCTTCTCCGGC |
| | CGCTCCTGCGCCAAGTGCGCCGGCG |
| 105 | GCGTCTCCCGCGGCACCGGCTCCGGCTTCTCCAGC |
| | GGCGCCTGCGCCGTCTGCTCCGGCG |
| 106 | GCTAGTCCAGCGGCACCGGCGCCGGCTAGTCCGGC |
| | TGCTCCCGCTCCATCTGCACCGGCG |
| 107 | GCTTCTCCGGCTGCCCCGGCGCCGGCATCTCCAGC |
| | GGCGCCCGCACCTAGCGCCCCGGCG |
| 108 | GCTTCTCCGGCCGCCCCGGCGCCGGCTAGCCCAGC |
| | GGCGCCTGCGCCGAGTGCGCCGGCG |
| 109 | GCGAGTCCTGCGGCCCCAGCGCCGGCTTCTCCGGC |
| | AGCTCCTGCGCCAAGCGCTCCGGCG |
| 110 | GCTAGTCCTGCGGCTCCCGCGCCGGCTAGTCCAGC |
| | GGCGCCCGCCCCTAGTGCGCCGGCG |
| 111 | GCGAGCCCTGCTGCTCCCGCGCCGGCTAGCCCGGC |
| | TGCTCCTGCGCCGTCTGCCCCGGCG |

The amino acid repeat used in a random coil PAS polypeptide domain, in one embodiment, comprises at least 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues, wherein the amino acid repeat comprises at least one Ala, Ser, and Pro residue. In one embodiment, the amino acid repeat does not comprise more than 100 amino acid residues. The amino acid repeat, in one embodiment, comprises at least about 4%, at least about 5%, at least about 6%, at least about 10%, at least about 15%, or at least about 20% Pro residues. In a further embodiment, the amino acid repeat comprises less than about 40%, e.g., less than about 35% Pro residues.

In one embodiment, the random coil polypeptide domain comprises no more than 5 identical consecutive amino acid residues, e.g., no more than 4 identical consecutive amino acid residues, e.g., no more than 3 identical consecutive amino acid residues.

In one embodiment, the random coil polypeptide domain comprises more than about 4% Ala residues but less than about 50% Ala residues, e.g., more than about 10% Ala residues but less than about 50% Ala residues, e.g., more than about 20% Ala residues but less than about 50% Ala residues.

In another embodiment, the random coil polypeptide domain comprises more than about 4% Ser residues but less than about 50% Ser residues, e.g., more than about 10% Ser residues but less than about 50% Ser residues, e.g., more than about 20% Ser residues but less than about 50% Ser residues.

In one embodiment, the random coil polypeptide domain comprises about 35% Pro residues, about 50% Ala residues and about 15% Ser residues. Alternatively, the random coil polypeptide domain comprises about 35% Pro residues, about 15% Ala residues and about 50% Ser residues.

In one embodiment of the invention, the uricase conjugate comprises a random coil domain comprising the amino acid sequence set forth in SEQ ID NO: 60.

In one embodiment of the invention, the uricase conjugate comprises a random coil domain comprising the amino acid sequence set forth in SEQ ID NO: 61.

In one embodiment of the invention, the uricase conjugate comprises a random coil domain comprising the amino acid sequence set forth in SEQ ID NO: 62.

In one embodiment of a random coil domain provided herein, where the random coil domain is present at the N-terminus of the fusion protein, the random coil domain includes an N-terminal Met residue. In another embodiment, the fusion protein does not include an N-terminal Met residue, e.g., because it was removed after translation.

In one embodiment, the uricase conjugate of the present invention is a fusion protein. A fusion protein as described herein comprises at least one uricase domain and at least one random coil polypeptide domain in a multi-domain polypeptide. In an alternative embodiment, the uricase is bonded via a non-peptide bond to the random coil polypeptide domain. Non-peptide bonds that are useful for cross-linking proteins are known in the art and may include disulfide bonds, e.g., between Cys side chains, thioether bonds or non-peptide covalent bonds induced by chemical cross-linkers, such as disuccinimidyl suberate (DSS) or sulfosuccinimidyl 4-[pmaleimidophenyl] butyrate (Sulfo-SMPB), as well as non-covalent protein-protein interactions.

With respect to the fusion protein embodiments, the two domains can be arranged in an order selected by the ordinary skilled artisan. For example, in one embodiment of a uricase conjugate that is a fusion protein, the uricase domain is located at the amino (N—) terminus of the fusion protein and the random coil polypeptide domain is located at the carboxy (C—) terminus of the fusion protein. However, this order may also be reversed, e.g., in one embodiment, the uricase domain is located in/at the carboxy (C—) terminus and the random coil polypeptide domain is located in/at the amino (N—) terminus of the fusion protein. In yet another embodiment, the random coil polypeptide domain is located at both the C-terminus and N-terminus of the fusion protein, and the uricase domain is in between both random coil domains.

In one embodiment, the uricase fusion protein includes an N-terminal Met residue. In another embodiment, the uricase fusion protein does not include an N-terminal Met residue, e.g., because it was removed after translation. As such, for the sequences provided herein, if an N-terminal Met is present, alternative embodiments include fusion proteins of the same sequences that do not include the N-terminal Met. Similarly, if an N-terminal Met is not present in a fusion protein provided herein, alternative embodiments include fusion proteins of the same sequences that have the N-terminal Met present.

In one embodiment of a uricase fusion protein, an amino acid spacer sequence is present between the uricase domain and the random coil (e.g., PAS) domain. The amino acid spacer sequence in one embodiment, is one amino acid long, two amino acids long, three amino acids long or four amino acids long. In a further embodiment, the amino acid spacer sequence is two amino acids long. In a further embodiment, the spacer sequence is Gly-Ser.

In one embodiment of the uricase conjugate described herein, the conjugate comprises a purification tag at the C-terminus, N-terminus or both the N- and C-terminus. The purification tag is employed to facilitate purification of the uricase conjugate (i.e., the uricase fusion protein) from an in vitro expression system, for example, via the use of immobilized metal affinity chromatography (IMAC). The purification tag, in one embodiment, is present at the C-terminus of the uricase fusion protein. In a further embodiment, the purification tag is a polyhistidine tag (also referred to as a “his-tag”). The his-tag, in one embodiment, comprises six (6) histidine residues.

Alternative purification tags can also be employed herein. For example, in one embodiment, a his-glu tag (HQ tag) is present at the C-terminus of one of the uricase conjugates described herein. In a further embodiment, the HQ tag has the amino acid sequence HQHQHQ (SEQ ID NO:71). In another embodiment, the uricase conjugate comprises a his-asp tag (HN tag) at the C-terminus, to allow for purification of the conjugate. The HN tag, in one embodiment, has the amino acid sequence: HNHNHNHNHNHN (SEQ ID NO:72). In yet another embodiment, the uricase conjugate comprises a HAT peptide tag at the C-terminus, to allow for purification of the conjugate. The HAT peptide tag, in one embodiment, has the amino acid sequence: KDHLIHNVHKEEHAHAHNK (SEQ ID NO:73).

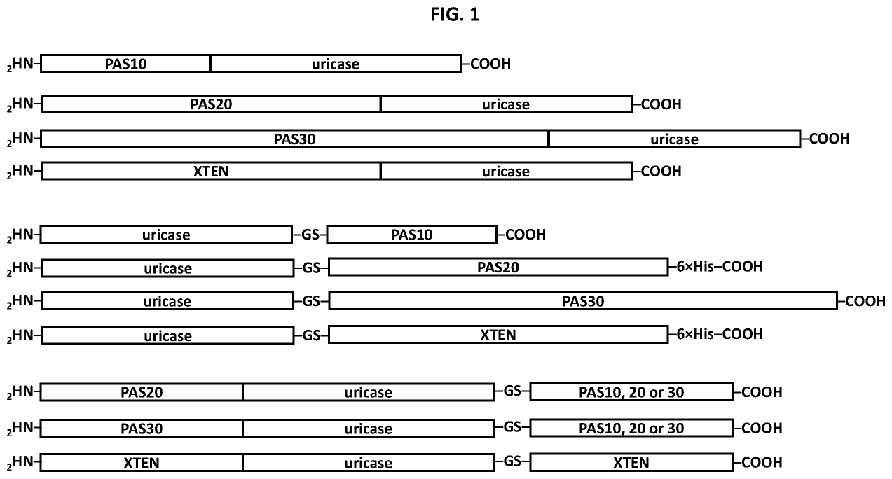

For the fusion protein embodiments of the uricase conjugate of the present disclosure, exemplary configurations of a recombinant uricase fusion protein comprising a uricase domain and one or two random coil PAS polypeptide or XTEN polypeptide domains are provided in FIG. 1. In one embodiment, the uricase fusion protein includes the uricase domain and one random coil PAS polypeptide or XTEN polypeptide domain. In the uricase fusion protein, the uricase domain may be C-terminal to the one random coil PAS polypeptide or XTEN polypeptide domain, or may be N-terminal to the one random coil PAS polypeptide or XTEN polypeptide domain. In another embodiment, the uricase fusion protein includes the uricase domain and two random coil PAS polypeptide domains or two XTEN polypeptide domains. In the uricase fusion protein, one of the two random coil PAS polypeptide or XTEN polypeptide domains is at the N-terminus and the other is at the C-terminus, and the uricase domain is between the two random coil PAS polypeptide or XTEN polypeptide domains. In some embodiments, the spacer sequence Gly-Ser (GS) between the uricase domain and a C-terminal random coil PAS polypeptide (e.g., PAS10, PAS20, or PAS30) or XTEN polypeptide domain, as depicted in FIG. 1, is optional and can be absent. In one embodiment, the uricase fusion protein has a polyhistidine tag (also referred to as a “his-tag”) comprising, e.g., six histidine residues, at the C-terminus, the N-terminus, or both the C-terminus and the N-terminus for purification of the uricase fusion protein. In some embodiments, the his-tag is separated from its adjacent domain by the spacer sequence Gly-Ser (GS).

PAS10, PAS20, and PAS30 represent random coil PAS polypeptide domains respectively comprising 10, 20, and 30 tandem copies of a PAS sequence comprising Pro, Ala, and Ser, such as the PAS sequence set forth in SEQ ID NO: 48, SEQ ID NO: 50, SEQ ID NO: 52, SEQ ID NO: 54, SEQ ID NO: 56 or SEQ ID NO: 58.

In one embodiment, PAS10, PAS20, and PAS30 represent random coil PAS polypeptide domains respectively comprising 10, 20, and 30 tandem copies of the PAS sequence of SEQ ID NO: 48, thereby having the amino acid sequences as set forth in SEQ ID NO: 60, SEQ ID NO:61, and SEQ ID NO:62, respectively.

In one embodiment, the DNA sequences encoding PAS10 of SEQ ID NO:60, PAS20 of SEQ ID NO:61, and PAS30 of SEQ ID NO:62 respectively comprise a total of 10, 20, and 30 tandem copies of one or more nucleotide sequences selected from the group consisting of SEQ ID NO:49 and SEQ ID NOs: 81-111.

In one embodiment, the DNA sequences encoding PAS10 of SEQ ID NO:60, PAS20 of SEQ ID NO:61, and PAS30 of SEQ ID NO:62 respectively comprise a total of 10, 20, and 30 tandem copies of the same (one) nucleotide sequence selected from the group consisting of SEQ ID NO:49 and SEQ ID NOs: 81-111.

In one embodiment, the DNA sequences encoding PAS10 of SEQ ID NO:60, PAS20 of SEQ ID NO:61, and PAS30 of SEQ ID NO:62 respectively comprise a total of 10, 20, and 30 tandem copies of two or more nucleotide sequences selected from the group consisting of SEQ ID NO:49 and SEQ ID NOs: 81-111.

In one embodiment, the DNA sequences encoding PAS10 of SEQ ID NO:60, PAS20 of SEQ ID NO:61, and PAS30 of SEQ ID NO:62 respectively comprise 10, 20, and 30 unique (i.e., different) nucleotide sequences selected from the group consisting of SEQ ID NO: 49 and SEQ ID NOs: 81-111 tandemly joined together via 3′,5′-phosphodiester bonds.

In an exemplary embodiment, the DNA sequence encoding PAS10 of SEQ ID NO: 60 comprises, in the 5′ to 3′ direction, each of SEQ ID NOs: 102-111 tandemly joined together in the ascending ID numerical order via 3′,5′-phosphodiester bonds.

In an exemplary embodiment, the DNA sequence encoding PAS20 of SEQ ID NO: 61 comprises, in the 5′ to 3′ direction, each of SEQ ID NOs: 92-111 tandemly joined together in the ascending ID numerical order via 3′,5′-phosphodiester bonds.

In an exemplary embodiment, the DNA sequence encoding PAS30 of SEQ ID NO: 62 comprises, in the 5′ to 3′ direction, each of SEQ ID NOs: 82-111 tandemly joined together in the ascending ID numerical order via 3′,5′-phosphodiester bonds.

In another exemplary embodiment, the DNA sequence encoding PAS30 of SEQ ID NO: 62 comprises, in the 5′ to 3′ direction, each of SEQ ID NO:81 and SEQ ID NOs: 83-111 tandemly joined together in the ascending ID numerical order via 3′,5′-phosphodiester bonds.

In one embodiment, the XTEN polypeptide domain of the uricase fusion protein comprises the amino acid sequence of SEQ ID NO:74.

In one embodiment, the uricase domain of the uricase fusion protein comprises a chimeric pig-baboon uricase of SEQ ID NO:1. In one embodiment, the uricase domain of the uricase fusion protein comprises a chimeric pig-baboon uricase of SEQ ID NO:4. In one embodiment, the uricase domain of the uricase fusion protein comprises a chimeric pig-baboon uricase of SEQ ID NO:40 (also referred to as “CPB40 uricase” herein). In one embodiment, the uricase domain of the uricase fusion protein comprises a chimeric pig-baboon uricase of SEQ ID NO:41 (also referred to as “CPB41 uricase” herein). In one embodiment, the uricase domain of the uricase fusion protein comprises a chimeric pig-baboon uricase of SEQ ID NO: 42. In one embodiment, the uricase domain of the uricase fusion protein comprises a chimeric pig-baboon uricase of SEQ ID NO:43.

Table 3 shows the code names, SEQ ID NOs and domain compositions of exemplary uricase fusion proteins which the inventors of the present application have created and produced in E. coli. Table 3 also describes the DNA sequences encoding PAS10 of SEQ ID NO: 60, PAS20 of SEQ ID NO:61, and PAS30 of SEQ ID NO:62. The exemplary uricase fusion proteins comprise the amino acid sequence of either the CPB40 uricase or CPB41 uricase for the uricase domain in combination with one or two random coil PAS polypeptide domains, or one random coil XTEN polypeptide domain, according to the configurations of FIG. 1 or their variations disclosed above. Details of the creation and characterization of some of the exemplary uricase fusion proteins are described in the “Example.”

TABLE 3
Code names, SEQ ID NOs and domain compositions of exemplary uricase fusion proteins

| | SEQ | |
|---|---|---|
| | ID | |
| Code Name | NO | Domain Composition from N-terminus to C-terminus |
| CPB40 uricase-CPAS10h | 63 | (CPB40 uricase)-(Gly-Ser spacer sequence)-(C-terminal PAS10 |
| | | of SEQ ID NO: 60)-(C-terminal his-tag comprising six histidine |
| | | residues) |
| | | The DNA sequence encoding C-terminal PAS10 of SEQ ID |
| | | NO: 60 comprises, in the 5′ to 3′ direction, each of SEQ ID |
| | | NOs: 102-111 tandemly joined together in the ascending ID |
| | | numerical order via 3′,5′-phosphodiester bonds. |
| CPB40 uricase-CPAS20h | 64 | (CPB40 uricase)-(Gly-Ser spacer sequence)-(C-terminal PAS20 |
| | | of SEQ ID NO: 61)-(C-terminal his-tag comprising six histidine |
| | | residues) |
| | | The DNA sequence encoding C-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| CPB40 uricase-CPAS30h | 65 | (CPB40 uricase)-(Gly-Ser spacer sequence)-(C-terminal PAS30 |
| | | of SEQ ID NO: 62)-(C-terminal his-tag comprising six histidine |
| | | residues) |
| | | The DNA sequence encoding C-terminal PAS30 of SEQ ID |
| | | NO: 62 comprises, in the 5′ to 3′ direction, each of SEQ ID |
| | | NOs: 82-111 tandemly joined together in the ascending ID |
| | | numerical order via 3′,5′-phosphodiester bonds. |
| CPB40 uricase-CXTENh | 66 | (CPB40 uricase)-(Gly-Ser spacer sequence)-(C-terminal XTEN |
| | | polypeptide of SEQ ID NO: 74)-(C-terminal his-tag comprising |
| | | six histidine residues) |
| NPAS10-CPB41 uricase-h | 67 | (N-terminal PAS10 of SEQ ID NO: 60)-(CPB41 uricase)-(Gly-Ser |
| | | spacer sequence)-(C-terminal his-tag comprising six histidine |
| | | residues) |
| | | The DNA sequence encoding N-terminal PAS10 of SEQ ID |
| | | NO: 60 comprises, in the 5′ to 3′ direction, each of SEQ ID |
| | | NOs: 102-111 tandemly joined together in the ascending ID |
| | | numerical order via 3′,5′-phosphodiester bonds. |
| NPAS20-CPB41 uricase-h | 68 | (N-terminal PAS20 of SEQ ID NO: 61)-(CPB41 uricase)-(Gly-Ser |
| | | spacer sequence)-(C-terminal his-tag comprising six histidine |
| | | residues) |
| | | The DNA sequence encoding N-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| NPAS30-CPB41 uricase-h | 69 | (N-terminal PAS30 of SEQ ID NO: 62)-(CPB41 uricase)-(Gly- |
| | | Ser spacer sequence)-(C-terminal his-tag comprising six histidine |
| | | residues) |
| | | The DNA sequence encoding N-terminal PAS30 of SEQ ID |
| | | NO: 62 comprises, in the 5′ to 3′ direction, each of SEQ ID |
| | | NOs: 82-111 tandemly joined together in the ascending ID |
| | | numerical order via 3′,5′-phosphodiester bonds. |
| NXTENh-CPB41 uricase-h | 70 | (N-terminal his-tag comprising six histidine residues)-(N-terminal |
| | | XTEN polypeptide of SEQ ID NO: 74)-(CPB41 uricase)-(Gly-Ser |
| | | spacer sequence)-(C-terminal his-tag comprising six histidine |
| | | residues) |
| NPAS20h-CPB41 uricase- | 75 | (N-terminal his-tag comprising six histidine residues)-(N-terminal |
| CPAS20h | | PAS20 of SEQ ID NO: 61)-(CPB41 uricase)-(Gly-Ser spacer |
| | | sequence)-(C-terminal PAS20 of SEQ ID NO: 61)-(C-terminal his- |
| | | tag comprising six histidine residues) |
| | | The DNA sequence encoding each of N-and C-terminal PAS20 |
| | | of SEQ ID NO: 61 comprises, in the 5′ to 3′ direction, each of |
| | | SEQ ID NOs: 92-111 tandemly joined together in the ascending |
| | | ID numerical order via 3′,5′-phosphodiester bonds. |
| NPAS20h-CPB41 uricase- | 76 | (N-terminal his-tag comprising six histidine residues)-(N-terminal |
| CPAS30h | | PAS20 of SEQ ID NO: 61)-(CPB41 uricase)-(Gly-Ser spacer |
| | | sequence)-(C-terminal PAS30 of SEQ ID NO: 62)-(C-terminal his- |
| | | tag comprising six histidine residues) |
| | | The DNA sequence encoding N-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| | | The DNA sequence encoding C-terminal PAS30 of SEQ ID |
| | | NO: 62 comprises, in the 5′ to 3′ direction, each of SEQ ID |
| | | NO: 81 and SEQ ID NOs: 83-111 tandemly joined together in the |
| | | ascending ID numerical order via 3′,5′-phosphodiester bonds. |
| NPAS10h-CPB41 uricase-h | 77 | (N-terminal his-tag comprising six histidine residues)-(N-terminal |
| | | PAS10 of SEQ ID NO: 60)-(CPB41 uricase)-(Gly-Ser spacer |
| | | sequence)-(C-terminal his-tag comprising six histidine residues) |
| | | The DNA sequence encoding N-terminal PAS10 of SEQ ID |
| | | NO: 60 comprises, in the 5′ to 3′ direction, each of SEQ ID |
| | | NOs: 102-111 tandemly joined together in the ascending ID |
| | | numerical order via 3′,5′-phosphodiester bonds. |
| NPAS30h-CPB41 uricase-h | 78 | (N-terminal his-tag comprising six histidine residues)-(N-terminal |
| | | PAS30 of SEQ ID NO: 62)-(CPB41 uricase)-(Gly-Ser spacer |
| | | sequence)-(C-terminal his-tag comprising six histidine residues) |
| | | The DNA sequence encoding N-terminal PAS30 of SEQ ID |
| | | NO: 62 comprises, in the 5′ to 3′ direction, each of SEQ ID |
| | | NOs: 82-111 tandemly joined together in the ascending ID |
| | | numerical order via 3′,5′-phosphodiester bonds. |
| NXTEN-CPB41 uricase-h | 79 | (N-terminal XTEN polypeptide of SEQ ID NO: 74)-(CPB41 |
| | | uricase)-(Gly-Ser spacer sequence)-(C-terminal his-tag |
| | | comprising six histidine residues) |
| NPAS20h-CPB41 uricase-h | 80 | (N-terminal his-tag comprising six histidine residues)-(N-terminal |
| | | PAS20 of SEQ ID NO: 61)-(CPB41 uricase)-(Gly-Ser spacer |
| | | sequence)-(C-terminal his-tag comprising six histidine residues) |
| | | The DNA sequence encoding N-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| NXTEN-CPB41 uricase- | 112 | (N-terminal XTEN polypeptide of SEQ ID NO: 74)-(pig-baboon |
| CXTEN | | chimeric uricase of SEQ ID NO: 41)-(C-terminal XTEN |
| | | polypeptide of SEQ ID NO: 74) |
| NPAS20-CPB41 uricase- | 113 | (N-terminal PAS20 of SEQ ID NO: 61)-(pig-baboon chimeric |
| CPAS20h | | uricase of SEQ ID NO: 41)-(Gly-Ser spacer sequence)-(C-terminal |
| | | PAS20 of SEQ ID NO: 61)-(C-terminal his-tag comprising six |
| | | histidine residues) |
| | | The DNA sequence encoding each of N-and C-terminal PAS20 |
| | | of SEQ ID NO: 61 comprises, in the 5′ to 3′ direction, each of |
| | | SEQ ID NOs: 92-111 tandemly joined together in the ascending |
| | | ID numerical order via 3′,5′-phosphodiester bonds. |

In one aspect, the present invention relates to nucleic acid constructs which encode the uricase conjugates (fusion proteins) of the invention. The nucleic acid molecules may be operably linked to suitable expression control sequences known in the art to ensure proper transcription and translation of the polypeptide as well as signal sequences to ensure cellular secretion or targeting to organelles. Such vectors may comprise further genes such as marker genes which allow for the selection of said vector in a suitable host cell and under suitable conditions.

A “nucleic acid construct” refers to a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, two or more operably linked proteins (e.g., to produce fusion proteins), and the like.

“Operably linked” refers to an arrangement of elements wherein the components so described are configured so as to perform their usual function. In the case of a promoter, a promoter that is operably linked to a coding sequence will affect the expression of a coding sequence. The promoter or other control elements need not be contiguous with the coding sequence, so long as they function to direct the expression thereof. For example, intervening untranslated yet transcribed sequences can be present between the promoter sequence and the coding sequence, and the promoter sequence can still be considered “operably linked” to the coding sequence. In the case of the different domains of the uricase conjugate described herein, the domains can be operably linked in a single DNA sequence to allow for expression of the uricase fusion protein.

A “vector” is capable of transferring gene sequences to a host cell. A “vector” refers to a nucleic acid construct capable of directing the expression of a gene of interest and which can transfer gene sequences to host cells, which can be accomplished by genomic integration of all or a portion of the vector, or transient or inheritable maintenance of the vector as an extrachromosomal element. Thus, the term includes cloning, and expression vectors, as well as integrating vectors. Vectors for use herein include plasmids, phage, phagemids, adenoviral, adeno-associated virus (AAV), lentiviral, for example.

In one embodiment, the nucleic acid construct is present in a recombinant vector in which a nucleic acid molecule encoding the uricase fusion protein, is operatively linked to expression control sequences allowing expression of the uricase fusion protein in prokaryotic or eukaryotic cells.

Expression of the nucleic acid molecule comprises transcription of the nucleic acid molecule into a translatable mRNA. Regulatory elements permitting expression in prokaryotic host cells comprise, e.g., the lambda PL, lac, trp, tac, tet or T7 promoter in E. coli. Possible regulatory elements ensuring expression in eukaryotic cells, for example, in mammalian cells or yeast, are well known to those skilled in the art. In one embodiment, the regulatory element comprises regulatory sequences ensuring initiation of transcription and optionally poly-A signals ensuring termination of transcription and stabilization of the transcript. Additional regulatory elements for use herein, include transcriptional as well as translational enhancers, and/or naturally associated or heterologous promoter regions. Examples of regulatory elements permitting expression in eukaryotic host cells are the AOXI or GALI promoter in yeast or the CMV, SV40, RSV promoter (Rous sarcoma virus), CMV enhancer, SV40 enhancer or a globin intron in mammalian and other animal cells. Apart from elements which are responsible for the initiation of transcription such regulatory elements may also comprise transcription termination signals, such as the SV40-poly-A site or the tk-poly-A site, downstream of the coding region.

Methods which are well known to those skilled in the art can be used to construct recombinant vectors (see, for example, the techniques described in Sambrook (1989), Molecular Cloning: A Laboratory Manual, Cold Spring Harbor Laboratory N. Y. and Ausubel (1989), Current Protocols in Molecular Biology, Green Publishing Associates and Wiley Interscience, N.Y). In this context, suitable expression vectors are known in the art such as Okayama-Berg cDNA expression vector pcDVI (Pharmacia), pCDM8, pRc/CMV, pcDNA1, pcDNA3, pPICZalpha A (Invitrogen), or pSPORT1 (GIBCO BRL). Furthermore, depending on the expression system that is used, leader sequences capable of directing the polypeptide to a cellular compartment or secreting it into the culture medium may be added to the coding sequence of the nucleic acid molecule of the invention.

The present invention also relates in certain embodiments, to vectors, particularly plasmids, cosmids, viruses, and bacteriophages that are conventionally employed in genetic engineering comprising a nucleic acid construct encoding the fusion proteins provided herein. The vector, in one embodiment, is an expression vector. Expression vectors derived from viruses such as retroviruses, vaccinia virus, adeno-associated virus, herpes viruses or bovine papilloma virus may be used for delivery of the polynucleotides or vector of the invention into targeted cell populations. The vectors containing the nucleic acid molecules of the invention can be transferred into the host cell by well-known methods, which vary depending on the type of cellular host. Accordingly, the invention further relates to a cell comprising the nucleic acid molecule or the vector.

In one embodiment, calcium chloride transfection, a commonly utilized for prokaryotic cells is used to transfect a host cell with one of the nucleic acid molecules/vectors provided herein. In another embodiment, calcium phosphate treatment or electroporation is used, depending on the cellular host. As a further alternative, the nucleic acid molecules and vectors of the invention can be reconstituted into liposomes for delivery to target cells. The nucleic acid molecule or vector of the invention which is present in host cell may either be integrated into the genome of the host cell or it may be maintained extrachromosomally. Accordingly, the present invention also relates in part to a host cell comprising the nucleic acid molecule and/or the vector of this invention. Host cells for the expression of polypeptides are well known in the art and comprise prokaryotic cells as well as eukaryotic cells, e.g., E. coli cells, yeast cells, invertebrate cells, CHO-cells, CHO-Kl-cells, Hela cells, COS-1 monkey cells, melanoma cells such as Bowes cells, mouse L-929 cells, 3T3 lines derived from Swiss, Balb-c or NIH mice, BHK or HaK hamster cell lines and the like.

In an additional aspect, the present invention comprises methods for the preparation of the uricase fusion protein of the invention comprising culturing a host cell and isolating the uricase fusion protein from the culture. The uricase fusion protein comprising a uricase domain and a random coil polypeptide domain may be produced by recombinant DNA technology, e.g., by culturing a cell comprising a nucleic acid construct or vector encoding the uricase fusion protein and isolating the uricase fusion protein from the culture. The uricase fusion protein may be produced in any suitable cell-culture system including prokaryotic cells, e.g., E. coli, BL21 or JM83, or eukaryotic cells, e.g., Pichia pastoris yeast strain X-33 or CHO cells. Further suitable cell lines known in the art are obtainable from cell line depositories, such as the American Type Culture Collection (ATCC). The term “prokaryotic” is meant to include bacterial cells while the term “eukaryotic” is meant to include yeast, higher plant, insect and mammalian cells. The transformed hosts can be grown in fermenters and cultured according to techniques known in the art to achieve optimal cell growth. In a further embodiment, the present invention relates in part to a process for the preparation of a uricase fusion protein described above comprising culturing a cell of the invention under conditions suitable for the expression of the uricase fusion protein and isolating the uricase fusion protein from the cell or the culture medium.

The uricase fusion protein can be isolated from the growth medium, cellular lysates or cellular membrane fractions. The isolation and purification of the expressed polypeptides of the invention may be performed by any conventional means, including ammonium sulphate precipitation, affinity columns, column chromatography, gel electrophoresis and the like and may involve the use of monoclonal or polyclonal antibodies directed, e.g., against a tag fused with the biologically active protein of the invention. For example, the protein can be purified via the Strep-tag II using streptavidin affinity chromatography (Skerra (2000). Methods Enzymol 326, pp. 271-304).

cDNA coding for the conjugate can be cloned and inserted into the appropriate vector, e.g., for expression in a suitable host such as E. coli or Saccharomyces cerevisiae.

In one aspect, the present disclosure provides a pharmaceutical composition comprising the uricase conjugate, which in some embodiments, is a uricase fusion protein disclosed herein. The pharmaceutical composition, in one embodiment, comprises, a pharmaceutically acceptable excipient, carrier, buffer, stabilizer or other materials well known to those skilled in the art. Such materials are non-toxic and should not interfere with the efficacy of the uricase enzyme. Such materials may include, for example, solvents, dispersion media, antibacterial and antifungal agents, isotonic and absorption delaying agents. Some examples of pharmaceutically acceptable carriers are water, saline, phosphate buffered saline, dextrose, glycerol, and ethanol, as well as combinations thereof. In one embodiment, the pharmaceutical composition includes an isotonic agent, for example, a sugar, and/or a polyalcohol, such as mannitol or sorbitol, or sodium chloride. Additional examples of pharmaceutically acceptable substances are wetting agents or auxiliary substances, such as emulsifying agents, preservatives or buffers, which increase the shelf life or effectiveness.

The pharmaceutical composition may be formulated in liquid, semi-solid or solid forms, such as liquid solutions (e.g., injectable and infusible solutions), dispersions or suspensions, powders, liposomes, and suppositories. The preferred form depends on the intended mode of administration, therapeutic application, the physicochemical properties of the uricase conjugate, and the route of delivery. Formulations may include excipients, or combinations of excipients, for example: sugars, amino acids and surfactants. Liquid formulations may include a wide range of protein concentrations and pH. Solid formulations may be produced by lyophilization, spray drying, or drying by supercritical fluid technology, for example.

For intravenous injection, or injection at the site of affliction, the active ingredient may be in a parenterally acceptable aqueous solution which is pyrogen-free and has suitable pK, isotonicity, and stability. Those of relevant skill in the art are well able to prepare suitable solutions using, for example, isotonic vehicles such as sodium chloride solution, Ringer's solution, and a lactated Ringer's solution. Preservatives, stabilizers, buffers, antioxidants and/or other additives may be included.

In some embodiments, the pharmaceutical composition is formulated as a solution, microemulsion, dispersion, liposome dispersion, or other ordered structure suitable to contain a uricase conjugate described herein (e.g., a uricase fusion protein). Sterile injectable solutions can be prepared by incorporating the uricase conjugate in an appropriate solvent with one or a combination of ingredients enumerated above, followed by filtered sterilization. Generally, dispersions are prepared by incorporating the uricase conjugate into a sterile vehicle that contains a dispersion medium and other ingredients from those enumerated above. In the case of sterile powders for the preparation of sterile injectable solutions, the methods of preparation include vacuum drying and freeze-drying that yields a powder of the active ingredient plus any additional desired ingredient from a previously sterile-filtered solution thereof. The proper fluidity of a solution can be maintained, for example, by using a coating such as lecithin, by maintaining the particle size of a dispersion, or by using surfactants. Prolonged absorption of injectable compositions can be brought about by including in the composition an agent that delays absorption, for example, monostearate salts and gelatin. In one embodiment, the uricase conjugate in the composition is a uricase fusion protein, as described herein.

In one embodiment, the pharmaceutical composition is a solution of a uricase conjugate, e.g., a phosphate buffered saline solution containing one of the uricase fusion proteins described herein. In a further embodiment, the solution is sterile and suitable for injection, e.g., intravenous injection or subcutaneous injection.

In another aspect, the present disclosure provides a method of reducing elevated uric acid levels, i.e., a method of treating hyperuricemia, in a subject in need thereof. The method includes administering to the subject an effective amount of the uricase conjugate or the pharmaceutical composition comprising the same.

The term “subject”, as used herein, refers to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A human subject may be an adult, a teenager, a child (2 years to 14 years of age), an infant (1 month to 24 months), or a neonate (up to 1 month). In one embodiment, the subject is an adult subject. In a further embodiment, the subject is an adult human subject or patient.

In one embodiment of the method, the uric acid levels are reduced in the plasma or blood of the subject. In one embodiment, suitable indicators for assessing effectiveness of the method include normalization or lowering of plasma uric acid levels (PUA), e.g., lowering or maintenance of PUA to 6.8 mg/dL or less, or 6 mg/dL or less in a human subject.

In some embodiments of the method, administration of the uricase conjugate or the pharmaceutical composition comprising the same, is carried out parenterally, e.g., via intramuscular, intrathecal, subcutaneous, or intravenous administration. In one embodiment, the administration is intravenous administration. In another embodiment, the administration is subcutaneous administration. In another embodiment, the administration is rectal, topical, or pulmonary administration.

In one embodiment, the subject is a gout patient. In one embodiment, the gout is recurrent gout. In another embodiment, the gout is advanced gout, with deposits of uric acid crystals forming under the skin in nodules called tophi. In a human subject, tophi can develop in several areas, such as fingers, hands, feet, elbows or Achilles tendons along the backs of ankles. In another embodiment, the subject with gout has kidney stones, which are uric acid crystals collected in the urinary tracts.

In one embodiment, the subject is a refractory gout patient, i.e., refractory to a prior different treatment. The prior treatment in one embodiment, is selected from treatment with nonsteroidal anti-inflammatory drugs (NSAIDs), colchicine, corticosteroids, allopurinol, febuxostat, probenecid, KRYSTEXXA® (pegloticase), rasburicase (Elitek®), or a combination of the foregoing. In one embodiment, refractory gout is a chronic condition characterized by high serum uric acid levels, recurrent gout flares, chronic arthritis, and progressive tophaceous deposition. In another embodiment, refractory gout is associated with high rates of cardiovascular and renal comorbidities.

In one embodiment, the subject has been diagnosed with tumor lysis syndrome. In a further embodiment, the subject has a lymphoma (e.g., a Burkitt's lymphoma, a non-Hodgkin lymphoma), acute lymphoblastic leukemia, or acute myeloid leukemia. In one embodiment, the subject diagnosed with tumor lysis syndrome is a human subject with a plasma uric acid concentration of >8 mg/dL. In another embodiment, the subject diagnosed with tumor lysis syndrome is a human subject with a plasma uric acid concentration of >15 mg/dL (hyperuricemia).

In one embodiment, the present disclosure provides a method of treating gout in a subject in need of treatment. The method includes administering to the subject an effective amount of the uricase conjugate described herein, or a pharmaceutical composition comprising the same. In one embodiment, the gout is refractory gout. In one embodiment, the gout is recurrent gout. In another embodiment, the gout is advanced gout, characterized by deposits of uric acid crystals forming under the skin in nodules called tophi. In another embodiment, the subject has kidney stones. In one embodiment, the subject is an adult subject. In a further embodiment, the subject is an adult human subject or patient.

The method for treating gout, in one embodiment, is carried out parenterally, e.g., via intramuscular, intrathecal, subcutaneous, or intravenous administration. In one embodiment, the administration is intravenous administration. In another embodiment, the administration is subcutaneous administration. In another embodiment, the administration is rectal, topical, or pulmonary administration.

In yet another embodiment of a method of treatment with the uricase conjugate described herein, the method is a method of treating tumor lysis syndrome in a subject in need of treatment. The method comprises administering to the subject an effective amount of the uricase conjugate or a pharmaceutical composition comprising the same. In one embodiment, the subject is an adult subject. In a further embodiment, the subject is an adult human subject or patient. In some embodiments of the method, administration of the uricase conjugate or the pharmaceutical composition comprising the same is carried out parenterally, e.g., via intramuscular, intrathecal, subcutaneous, or intravenous administration. In one embodiment, the administration is intravenous administration. In another embodiment, the administration is subcutaneous administration. In another embodiment, the administration is rectal, topical, or pulmonary administration. In some embodiments, the subject with tumor lysis syndrome has a lymphoma (e.g., a Burkitt's lymphoma, a non-Hodgkin lymphoma), acute lymphoblastic leukemia, or acute myeloid leukemia. In one embodiment, the subject with tumor lysis syndrome is a human subject with a plasma uric acid concentration of >8 mg/dL. In another embodiment, the subject with tumor lysis syndrome is a human subject with a plasma uric acid concentration of >15 mg/dL (hyperuricemia).

### Example

The present invention is further illustrated by reference to the following Example. However, it should be noted that the example, like the embodiments described above, is illustrative and is not to be construed as restricting the scope of the invention in any way.

### Example 1-Creation and Characterization of Uricase Fusion Proteins Comprising the CPB40 or CPB41 Uricase for the Uricase Domain in Combination with PAS or XTEN Polypeptide Domain

This example describes the creation and characterization of the uricase fusion proteins shown in Table 4. The uricase fusion proteins of CPB40 uricase-CPAS10h (SEQ ID NO: 63), CPB40 uricase-CPAS20h (SEQ ID NO:64), and CPB40 uricase-CPAS30h (SEQ ID NO: 65) each comprise the amino acid sequence of the CPB40 uricase for the uricase domain and one C-terminal PAS polypeptide domain. The uricase fusion protein of CPB40 uricase-CXTENh (SEQ ID NO:66) comprises the amino acid sequence of the CPB40 uricase for the uricase domain and one C-terminal XTEN polypeptide domain. The uricase fusion proteins of NPAS20 h-CPB41 uricase-CPAS20h (SEQ ID NO:75) and NPAS20 h-CPB41 uricase-CPAS30h (SEQ ID NO:76) each comprise the amino acid sequence of the CPB41 uricase for the uricase domain and two PAS polypeptide domains. In each of the uricase fusion proteins, one of the two PAS polypeptide domains is at the N-terminus and the other is at the C-terminus, and the uricase domain is between the two PAS polypeptide domains. Because the amino acid sequence of the CPB40 or CPB41 uricase is present in each protein monomer of PEGylated homotetrameric KRYSTEXXA® (pegloticase), KRYSTEXXA® (pegloticase) was used as a comparator in some studies described in this example. KRYSTEXXA® (pegloticase) is referred to as “pegloticase” only for brevity in this example.

TABLE 4
Code names, SEQ ID NOs and domain compositions of exemplified uricase fusion proteins

| | SEQ | |
|---|---|---|
| | ID | |
| Code Name | NO | Domain Composition from N-terminus to C-terminus |
| CPB40 uricase-CPAS10h | 63 | (CPB40 uricase)-(Gly-Ser spacer sequence)-(C-terminal PAS10 |
| | | of SEQ ID NO: 60)-(C-terminal his-tag comprising six histidine |
| | | residues) |
| | | The DNA sequence encoding C-terminal PAS10 of SEQ ID |
| | | NO: 60 comprises, in the 5′ to 3′ direction, each of SEQ ID |
| | | NOs: 102-111 tandemly joined together in the ascending ID |
| | | numerical order via 3′,5′-phosphodiester bonds. |
| CPB40 uricase-CPAS20h | 64 | (CPB40 uricase)-(Gly-Ser spacer sequence)-(C-terminal PAS20 |
| | | of SEQ ID NO: 61)-(C-terminal his-tag comprising six histidine |
| | | residues) |
| | | The DNA sequence encoding C-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| CPB40 uricase-CPAS30h | 65 | (CPB40 uricase)-(Gly-Ser spacer sequence)-(C-terminal PAS30 |
| | | of SEQ ID NO: 62)-(C-terminal his-tag comprising six histidine |
| | | residues) |
| | | The DNA sequence encoding C-terminal PAS30 of SEQ ID |
| | | NO: 62 comprises, in the 5′ to 3′ direction, each of SEQ ID |
| | | NOs: 82-111 tandemly joined together in the ascending ID |
| | | numerical order via 3′,5′-phosphodiester bonds. |
| CPB40 uricase-CXTENh | 66 | (CPB40 uricase)-(Gly-Ser spacer sequence)-(C-terminal XTEN |
| | | polypeptide of SEQ ID NO: 74)-(C-terminal his-tag comprising |
| | | six histidine residues) |
| NPAS20h-CPB41 uricase- | 75 | (N-terminal his-tag comprising six histidine residues)-(N-terminal |
| CPAS20h | | PAS20 of SEQ ID NO: 61)-(CPB41 uricase)-(Gly-Ser spacer |
| | | sequence)-(C-terminal PAS20 of SEQ ID NO: 61)-(C-terminal |
| | | his-tag comprising six histidine residues) |
| | | The DNA sequence encoding each of N-and C-terminal PAS20 |
| | | of SEQ ID NO: 61 comprises, in the 5′ to 3′ direction, each of |
| | | SEQ ID NOs: 92-111 tandemly joined together in the ascending |
| | | ID numerical order via 3′,5′-phosphodiester bonds. |
| NPAS20h-CPB41 uricase- | 76 | (N-terminal his-tag comprising six histidine residues)-(N-terminal |
| CPAS30h | | PAS20 of SEQ ID NO: 61)-(CPB41 uricase)-(Gly-Ser spacer |
| | | sequence)-(C-terminal PAS30 of SEQ ID NO: 62)-(C-terminal |
| | | his-tag comprising six histidine residues) |
| | | The DNA sequence encoding N-terminal PAS20 of SEQ ID |
| | | NO: 61 comprises, in the 5′ to 3′ direction, each of SEQ ID |
| | | NOs: 92-111 tandemly joined together in the ascending ID |
| | | numerical order via 3′,5′-phosphodiester bonds. |
| | | The DNA sequence encoding C-terminal PAS30 of SEQ ID |
| | | NO: 62 comprises, in the 5′ to 3′ direction, each of SEQ ID |
| | | NO: 81 and SEQ ID NOs: 83-111 tandemly joined together in the |
| | | ascending ID numerical order via 3′,5′-phosphodiester bonds. |

### Methods

### 1. Creation and Expression of the Uricase Fusion Proteins

DNA encoding the CPB40 or CPB41 uricase for the uricase domain of the fusion proteins set forth in Table 4, and DNA encoding the PAS polypeptide domain (i.e., PAS10, PAS20, and PAS30) or the XTEN polypeptide domain, with or without the his-tag and/or the GS spacer, were synthesized by Synbio Technologies (Monmouth Junction, NJ, USA). The resultant DNA fragments encoding the uricase domain, PAS or XTEN polypeptide domain were cloned into NdeI and BamHI digested expression vector pET-26b (+) (MilliporeSigma, MA, USA) using the NEBuilder® HiFi DNA Assembly Kit (New England Biolabs, Ipswich, MA, USA) to assemble the full-length coding sequence for each uricase fusion protein in the vector. The expression vector pET-26b (+) encodes a his-tag containing six histidine residues at the C-terminus of the assembled full-length uricase fusion protein coding sequence. Expression of the uricase fusion protein was achieved by growing E. coli cultures transformed with the expression vector pET-26b (+) containing the full-length uricase fusion protein coding sequence and inducing protein expression with 100 mM isopropyl β-D-1-thiogalactopyranoside (IPTG).

### 2. Purification

The uricase fusion proteins expressed in E. coli were purified to obtain soluble (non-aggregated) tetramers by the following procedures:
(1) pelleting 0.5 L IPTG-induced E. coli culture by centrifuging at 5,000×g for 10 minutes;
(2) resuspending the E coli pellet in 150 mL of 20 mM sodium borate buffer (pH 9.5);
(3) sonicating the resuspended pellet;
(4) centrifuging the sonicated suspension for 20 minutes at 17,000×g and decanting the soluble fraction;
(5) diluting the soluble fraction to 150 mL with 20 mM sodium borate buffer (pH 9.5) and stirring at 4° C.;
(6) during the stirring, slowly adding 19.5 g of ammonium sulfate solid;
(7) allowing the sample of step (6) to incubate at 4° C. for 2 hours;
(8) pelleting the sample of step (7) by centrifuging for 20 minutes at 17,000×g, and decanting soluble fraction;
(9) resuspending the protein pellet from step (8) in 40 mL of 20 mM sodium borate buffer (pH 9.5);
(10) desalting the suspension from step (9) using Zeba™ spin desalting columns (ThermoFisher Scientific);
(11) purifying the desalted sample from step (10) by loading the sample onto a 70 mL Toyopearl® NH2-75OF anion exchange column (Tosoh Bioscience) and eluting the purified sample with 140 mL of 20 mM sodium borate buffer (pH 9.5);
(12) performing buffer exchange of the eluate from step (11) into 1×PBS using a centrifuge spin-filter;
(13) loading the buffer exchanged sample from step (12) onto a Superose™ 6 size exclusion chromatography (SEC) column (Cytiva) to separate aggregates from soluble tetramer (target) proteins.

### 3. Determination of Enzymatic Activity of the Uricase Fusion Proteins and Pegloticase

The uricase enzymatic activity of the uricase fusion proteins and pegloticase (Serial #264790457565, Lot #0263A), reported as specific activity at the concentration of 72.8 nM and in units of μmol substrate (i.e., uric acid)/min/mg enzyme, was determined and calculated by the following procedures:
(1) diluting a uricase fusion protein or pegloticase sample to 1456.7 nM in 1× PBS (pH 7.4);
(2) in a 96-well plate, aliquoting 10 μL of the diluted uricase fusion protein or pegloticase sample per well;
(3) adding 190 μL of a uric acid substrate solution containing 0.125 mM uric acid to each well, bringing the final concentration of the uricase fusion protein or pegloticase to 72.8 nM in the reaction mixture;
(4) loading the 96-well plate onto a BIOTEK Synergy™ Neo2 plate reader and shaking the plate for 30 seconds;
(5) measuring absorbance at 293 nm every 30 seconds for 10 minutes under the controlled temperature of 25° C. on the plate reader;
(6) calculating Vmax values using the BIOTEK Gen5 software.

### 4. Differential Scanning Fluorimetry (DSF)

Differential scanning fluorimetry (DSF) measures the melting temperature of a natively folded protein sample with SYPRO™ Orange fluorescent dye. As the temperature is incrementally increased on a real-time PCR instrument, the fluorescent signal at λem 570 nm also increases, indicating exposure of hydrophobic residues and denaturation of the protein sample. Analyzing the inflection point(s) of the resulting curve yields the melting temperature (Tₘ), a measure of thermostability, for the protein.

To determine the Tₘ by DSF of the uricase fusion protein, the protein was diluted to a final concentration of 66-132 μg/mL in PBS buffer containing SYPRO™ Orange dye (Invitrogen, Cat #S6650) at a final concentration of 5×. Thirty μl of the protein-SYPRO™ Orange dye mixture was added to a well of an optical plate. Afterwards the optical plate was loaded onto a BioRad C1000 Touch™ Thermal Cycler with CFX96™ Real-Time System and exposed to a temperature gradient from 10° C. to 100° C. at an increment of 0.5° C. The Tₘ was calculated using gain of fluorescence of SYPRO™ orange and loss of fluorescence using PRISM software.

### 5. Dynamic Light Scattering

The uricase fusion proteins were subjected to dynamic light scattering (DLS) using a DynaPro® Plate Reader III (Waters Corporation) to determine each protein's hydrodynamic radius and aggregation status (via polydispersity measurement), as well as the percent mass in various mass ranges indicative of whether the protein was a soluble tetramer or was oligomerized or aggregated. Three mass ranges were selected: mass range 1 corresponding to 0.5-10 nm in hydrodynamic radius; mass range 2 corresponding to 10-100 nm in hydrodynamic radius; and mass range 3 corresponding to 100-1000 nm in hydrodynamic radius. Briefly, 100 μL of a protein sample with a concentration between 0.1 and 1 mg/ml was added to a well of a 96-well plate. The plate was centrifuged at 2,000×g for 5 min and loaded onto a DynaPro® Plate Reader III. The DLS data were collected with the following parameters:
(1) Experiment type-Isothermal;
(2) Enable auto-attenuation-Yes;
(3) Image each well-Yes;
(4) DLS acquisition time-10 seconds;
(5) DLS acquisitions per measurement-10;
(6) Measure SLS-No;
(7) Measurements per well within a scan-1;
(8) Wait between measurements within a scan-0 min;
(9) Number of Scans-1;
(10) Wait between scans-0 min;
(11) Start temp−25° C.;
(12) Plate sealant-no sealant;
(13) Wait for initial temperature lock-Yes;
(14) Set temperature−25° C.;
(15) Laser on-Yes.

### 6. In Vivo Pharmacokinetic (PK) and Efficacy Analyses of the Uricase Fusion Proteins and Pegloticase

Three PK studies were conducted. Purified soluble tetrameric uricase fusion proteins prepared as described in Section 2 above, as well as pegloticase serving as the comparator in PK studies 1 and 2, were subjected to PK and efficacy analyses in Wistar rats in vivo.

6.1. Dosing of Wistar Rats with the Uricase Fusion Proteins or Pegloticase and Collection of Rat Blood Samples

Female Wistar rats weighing about 220 g each were purchased from Charles River. In the PK studies described herein, the rats were either left untreated, or treated via intravenous administration with a single dose of pegloticase at 1 mg/kg body weight, or a single equimolar dose (equivalent to 1 mg/kg body weight of pegloticase) of a uricase fusion protein (n=3 rats for each group). 120 μL of blood was collected from each rat pre-dose as well as at various time points post-dose. Tables 5A-5C show the single dosage (in mg/kg rat body weight) of pegloticase or a uricase fusion protein administered to a Wistar rat and the blood sample collection timepoints selected in each of the three PK studies.

TABLE 5A
Single dosage of pegloticase or a uricase fusion protein administered
to Wistar rats and blood sample collection timepoints in PK study 1

| Pegloticase or uricase | Dosage | |
|---|---|---|
| fusion proteins tested in | (mg/kg | Blood sample |
| the study | body weight) | collection timepoints |
| Pegloticase | 1 | Pre-dose & 0.5 h, 2 h, |
| CPB40 uricase-CPAS10h | 1.51 | 6 h, 24 h, 72 h, and 96 h |
| CPB40 uricase-CPAS20h | 1.99 | post-dose |
| CPB40 uricase-CPAS30h | 2.47 | |
| CPB40 uricase-CXTENh | 2.16 | |

TABLE 5B
Single dosage of pegloticase or a uricase fusion protein administered
to Wistar rats and blood sample collection timepoints in PK study 2

| Pegloticase or uricase | Dosage | |
|---|---|---|
| fusion proteins tested | (mg/kg | Blood sample |
| in the study | body weight) | collection timepoints |
| Pegloticase | 1 | Pre-dose & 0.5 h, 1 h, |
| NPAS20h-CPB41 | 2.99 | 2 h, 6 h, 24 h, 48 h, 72 h |
| uricase-CPAS20h | | 96 h, 120 h, and 168 h |
| NPAS20h-CPB41 | 3.47 | post-dose |
| uricase-CPAS30h | | |

TABLE 5C
Single dosage of a uricase fusion protein administered to Wistar
rats and blood sample collection timepoints in PK study 3

| | Dosage | |
|---|---|---|
| Uricase fusion proteins | (mg/kg | Blood sample |
| tested in the study | body weight) | collection timepoints |
| CPB40 uricase-CPAS20h | 1.99 | Pre-dose & 1 h, 6 h, 24 h, |
| | | 48 h, 72 h, 96 h, 120 h, |
| NPAS20h-CPB41 | 2.99 | 168 h, 192 h, 216 h, |
| uricase-CPAS20h | | and 240 h post-dose |

### 6.2. Determination of Rat Plasma Concentrations of Uric Acid

The rat blood samples collected as described in Section 6.1 above were processed to obtain rat plasma, followed by determination of the plasma uric acid concentrations at each timepoint by liquid chromatography-tandem mass spectrometry (LC-MS/MS). The details for the quantification of uric acid concentrations in rat plasma are as follows.

### 6.2.1 Preparation of Artificial Human Plasma and Standard Uric Acid Samples Using the Artificial Human Plasma for Establishing a Standard Curve

Due to the presence of endogenous uric acid in rat plasma, artificial human plasma was used as the surrogate matrix for the preparation of standard uric acid samples for establishing a standard curve. The artificial human plasma was prepared by dissolving 2 g of human serum albumin (Sigma-Aldrich) in 50 mL PBS, followed by adjusting the pH to 7.4 with 1 M sodium hydroxide or 1 M phosphoric acid. Standard uric acid samples containing 0.1, 0.2, 1, 5, 10, 60, 80, and 100 μg/mL uric acid in the artificial human plasma were prepared and subjected to further processing and LC-MS/MS analyses in parallel with the study rat plasma samples as described below.

### 6.2.2. Processing of the Standard Uric Acid Samples and Rat Plasma Samples for LC-MS/MS

The standard uric acid samples of Section 6.2.1 and study rat plasma samples were processed according to the following procedures:
(1) adding 20 μl of 500 ng/ml uric acid-1,3-15N2 water solution (Sigma-Aldrich) and 40 μl of 0.4 N perchloric acid to 20 μl of a standard uric acid sample or a rat plasma sample;
(2) mixing and centrifuging the sample from step (1) at 15,000 rpm at 4° C. for 15 minutes using an Eppendorf 5424R centrifuge;
(3) pipetting out 50 μL supernatant from each sample following the centrifugation of step (2) and adding 50 μL water to the supernatant;
(4) mixing the sample obtained from step (3) for LC-MS/MS analysis described below.

### 6.2.3. LC-MS/MS Analyses of Standard Uric Acid Samples and Rat Plasma Samples to Quantify Uric Acid Concentrations

Ten μl of each standard uric acid sample or rat plasma sample processed as described in Section 6.2.2. was injected into an Acquity UPLC® BEH amide column of a Shimadzu Nexera™ LC (Sciex) followed by gradient elution. Uric acid quantification was performed using a triple quad API4500 mass spectrometer (Sciex) equipped with ESI operating in negative mode. The parameters for the mass spectrometer and LC are shown in Tables 6A and 6B, respectively.

TABLE 6A

| Parameters for Sciex API4500 mass spectrometer | |
|---|---|
| Curtain Gas (CUR) | 35 |
| Collision Gas (CAD) | 8 |
| Ion spray voltage (IS) | −4500 |
| Temperature (° C.) | 650 |
| Ion Source Gas 1 | 60 |
| Ion Source Gas 2 | 60 |
| Entrance potential | −10 |
| Collision Cell Exit Potential | −6 |

Ions Monitored in ESI Negative Mode

| Name | Q1 | Q3 | DP | CE |
|---|---|---|---|---|
| Uric acid | 167 | 123.8 | −30 | −21 |
| Uric acid-1,3-¹⁵N₂ | 168.9 | 124.9 | −30 | −21 |

TABLE 6B
LC conditions for uric acid

| | |
|---|---|
| Column | Acquity UPLC ® BEH amide 1.7 μm, 2.1 × 50 mm |
| Column temperature | 30° C. |
| Flow rate | 0.3 mL/min |
| Rinsing solution | 16.67% acetonitrile, 16.67% methanol, |
| | 16.67% isopropyl alcohol, 50% water |
| Mobile phase A | 0.1% formic acid in water |
| Mobile phase B | 0.1% formic acid in acetonitrile |

Isocratic Elution

| Time (min) | % A | % B |
|---|---|---|
| 0.0 | 10 | 90 |
| 1.0 | 10 | 90 |
| 2.5 | 60 | 40 |
| 3.0 | 60 | 40 |
| 3.1 | 10 | 90 |
| 4.0 | 10 | 90 |

LC-MS/MS data were collected with the data acquisition software Analyst™ 1.7 and MultiQuant™ 3.0 (Sciex) or their equivalents. The standard curve was generated with the data of the standard uric acid samples. Calibration curves were constructed by plotting the peak area ratio of the reference standard to the internal standard against the nominal concentration of the reference standard present. Calibration curves were fitted by least squares regression analysis to provide information on the slope of the calibration curve, the y axis intercept, the coefficient of correlation and the back-calculated calibration standard concentrations.

The standard curve obtained by using the standard uric acid samples prepared with the artificial human plasma was shown by a standard addition method to accurately measure uric acid concentrations in rat plasma. In the standard addition method, naïve rat plasma samples respectively spiked with three levels of uric acid concentration at 5, 25, and 80 μg/mL, as well as the blank naïve rat plasma sample (BioIVT), were processed and analyzed by LC-MS/MS as described above. Using the standard curve derived from the standard uric acid samples prepared with the artificial human plasma, the endogenous uric acid level in the blank naïve rat plasma and the uric acid levels in the three standard addition (i.e., uric acid-spiked) rat plasma samples were determined. The adjusted uric acid levels in the three standard addition samples after subtraction of the endogenous uric acid level were then compared with their target (spike) concentrations. The percentage differences between the adjusted and corresponding target uric acid concentrations were found to be less than 15%, meeting the acceptance criteria.

### 6.3. Determination of Rat Plasma Concentrations of the Uricase Fusion Proteins and Pegloticase

The rat blood samples collected as described in Section 6.1 above were processed to obtain rat plasma, followed by determination of the plasma concentrations of the uricase fusion proteins or pegloticase at each timepoint using the Amplex™ Red Uric Acid/Uricase Assay Kit (ThermoFisher Scientific, Cat #A22181). The assay kit provides a fluorometric method involving a two-step reaction to quantify the uricase fusion protein or pegloticase based on uricase enzymatic activity. First, the uricase enzymatic activity of the uricase fusion proteins or pegloticase converts uric acid to allantoin, hydrogen peroxide (H₂O₂) and carbon dioxide. Second, in the presence of horseradish peroxide (HRP), H₂O₂ stoichiometrically reacts with Amplex™ Red reagent to generate resorufin, a red fluorescent oxidation product. In the studies of this example, the rat plasma concentrations of the uricase fusion proteins or pegloticase were quantified indirectly by measuring the fluorescence of resorufin captured by a SpectraMax® mini plate reader (Molecular Devices) in kinetic mode for 30 minutes. Standard curves for quantifying the uricase fusion proteins or pegloticase were constructed using the same strain of rat plasma. The Excel add-in software XLift® by IDBS was used to process the standard curve regression with the equilibrium model and back calculate the uricase fusion protein or pegloticase concentrations in the rat plasma samples.

Rat plasma had endogenous uricase enzyme that also generated a fluorescence signal in the uricase assay. Additionally, the endogenous rat plasma uricase level fluctuated from rat to rat and over time within a single rat, making the determination of true background uricase infeasible for a given sample. As such, the determination of the plasma concentrations of the exogenously administered uricase fusion proteins or pegloticase in the study samples was approximate by subtracting the average pre-dose background (endogenous) plasma uricase concentration for each uricase fusion protein or pegloticase treated rat group. The plasma concentrations of the endogenous uricase in the untreated control rat group at the corresponding pre-dose timepoint (at time 0) and post-dose timepoints were likewise adjusted by subtracting the average pre-dose endogenous plasma uricase concentration of the control rat group.

### Results

### 1. Sodium Dodecyl-Sulfate Polyacrylamide Gel Electrophoresis (SDS-PAGE) Analysis

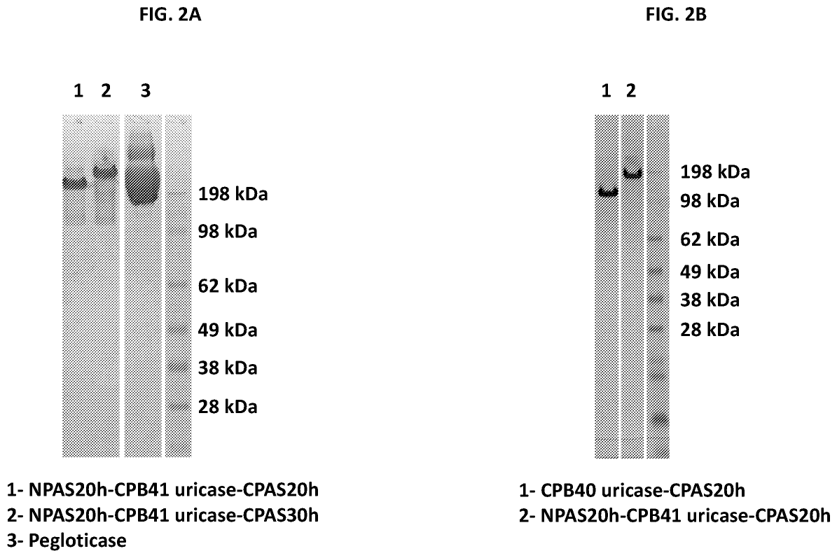

The uricase fusion proteins set forth in Table 4 were created, expressed in E. coli, and purified as soluble tetramers as described in the “Methods.” The purified uricase fusion proteins, together with pegloticase, were analyzed by SDS-PAGE under non-reducing conditions. FIGS. 2A and 2B are representative images of SDS-PAGE gels with bands representing monomeric uricase fusion proteins or monomeric pegloticase detected by Coomassie Brilliant Blue staining.

FIG. 2A shows the protein bands detected with the samples containing NPAS20 h-CPB41 uricase-CPAS20h, NPAS20 h-CPB41 uricase-CPAS30h, and pegloticase in lanes 1, 2, and 3, respectively. Those samples were used in PK study 2 of this example.

FIG. 2B shows the protein bands detected with the samples containing CPB40 uricase-CPAS20 h and NPAS20 h-CPB41 uricase-CPAS20 h in lanes 1 and 2, respectively. Those samples were used in PK study 3 of this example.

NPAS20 h-CPB41 uricase-CPAS20 h has an expected monomer molecular weight of 102650 Da. NPAS20 h-CPB41 uricase-CPAS30 h has an expected monomer molecular weight of 119168 Da. CPB40 uricase-CPAS20 h has an expected monomer molecular weight of 68328 Da. With the inclusion of a molecular weight standard shown in the rightmost lanes, the SDS-PAGE gels of FIGS. 2A and 2B revealed that the apparent sizes of the monomeric uricase fusion proteins based on the proteins' migration positions were notably greater than their corresponding expected sizes. This observation is consistent with the finding by Breibeck et al. that “[PAS polypeptide] fusion proteins with IL-1Ra [interleukin-1 receptor antagonist] and TrxA [E. coli thioredoxin] migrated at positions corresponding to much higher molecular weight than normally expected.” Breibeck et al. have postulated that “[the retarded electrophoretic mobility of PASylated proteins] is mostly explained by poor binding of SDS, whose negatively charged head groups provide the driving force in the electric field, probably due to the lack of any hydrophobic amino acid side chains.” See Breibeck et al., “The polypeptide biophysics of proline/alanine-rich sequences (PAS): Recombinant biopolymers with PEG-like properties,” Biopolymers. 2018 January; 109 (1): e23069, page 3, right column, the first paragraph.

2. In Vivo PK and Efficacy Analyses of the Uricase Fusion Proteins in Comparison with Pegloticase
2.1. PK Study 1 and Efficacy Analyses of Plasma Uric Acid Concentrations in Rat Blood Samples from the Study

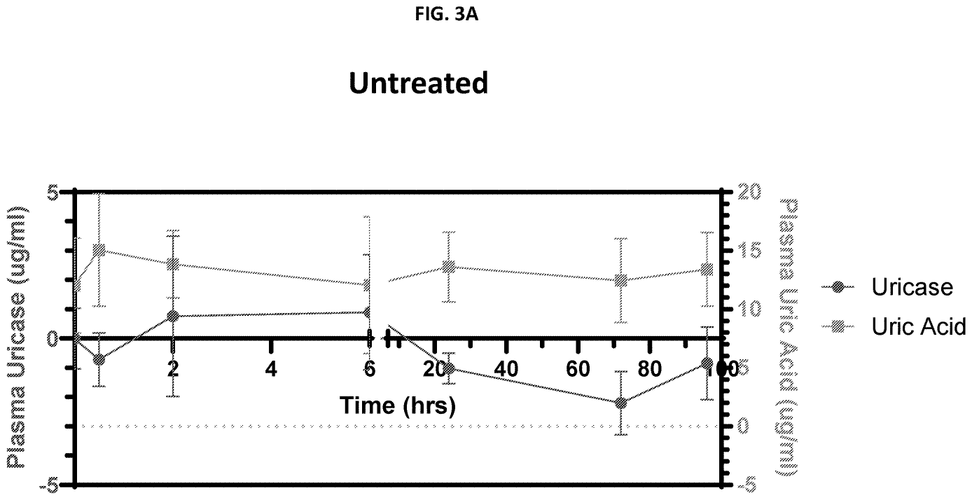

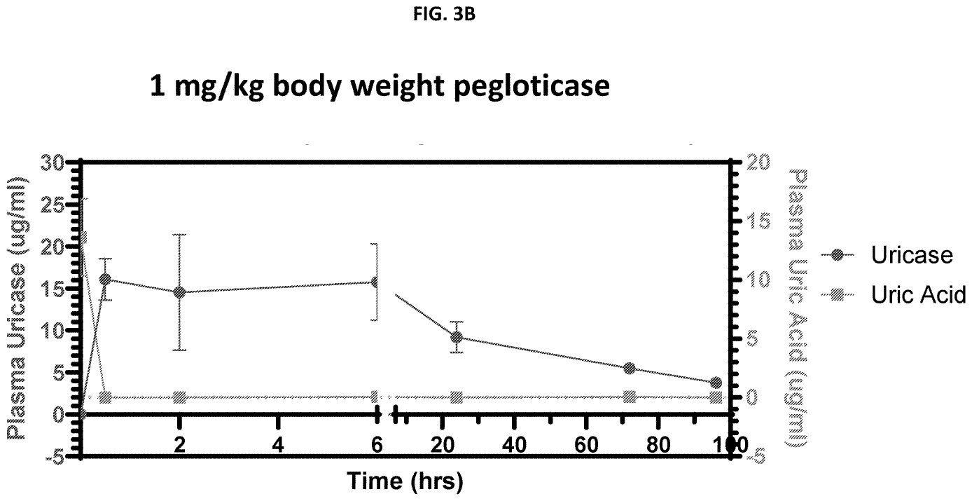

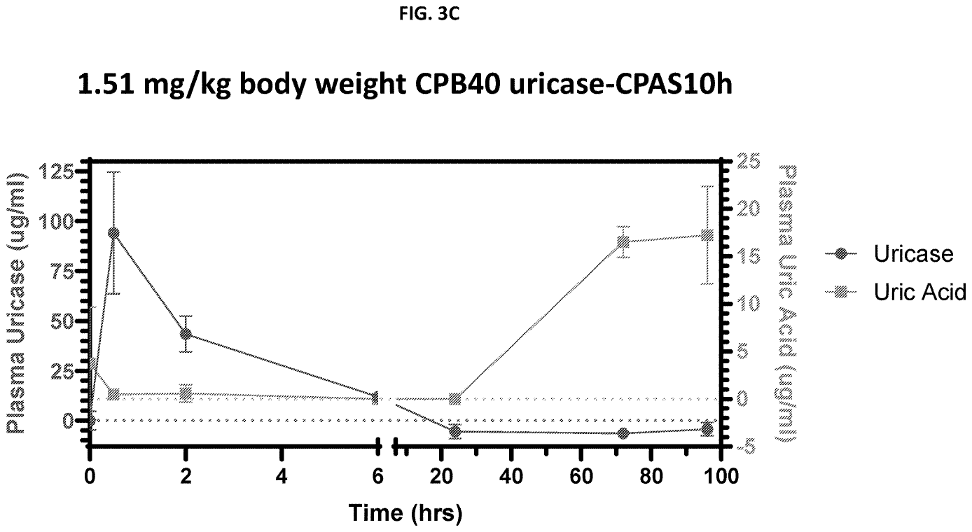

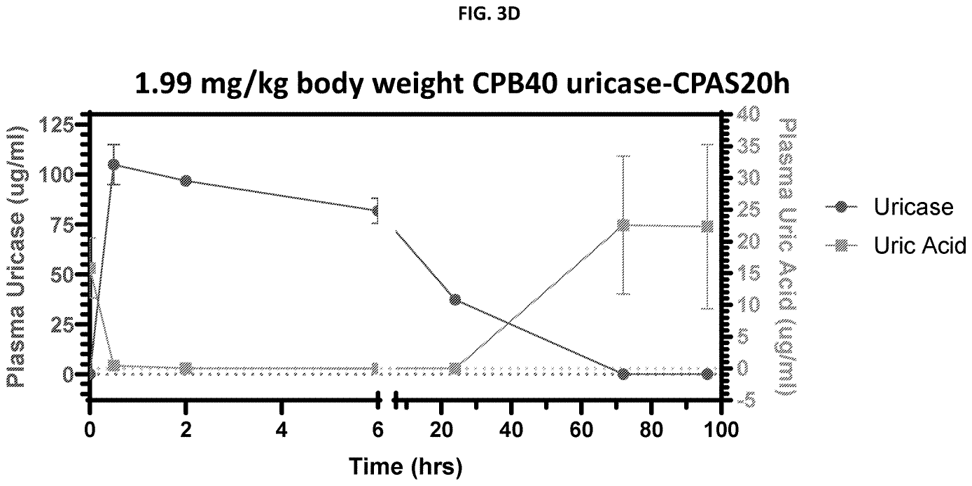

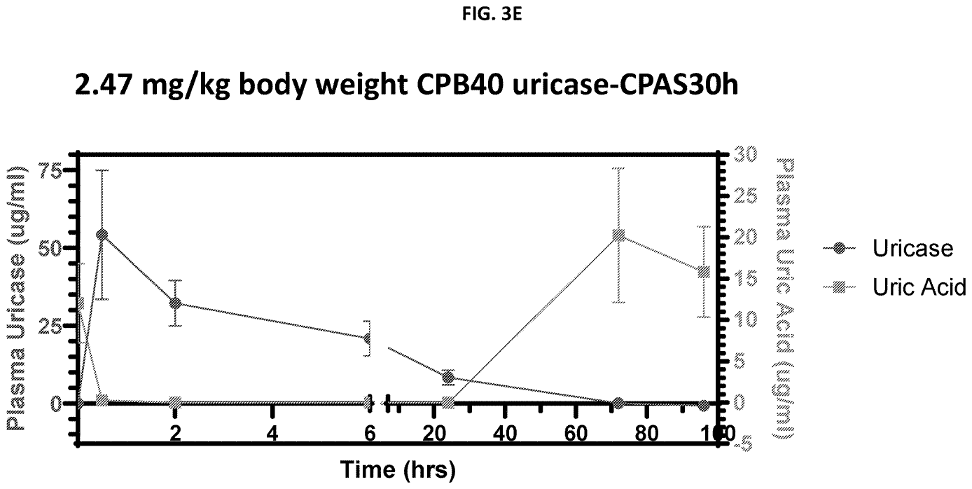

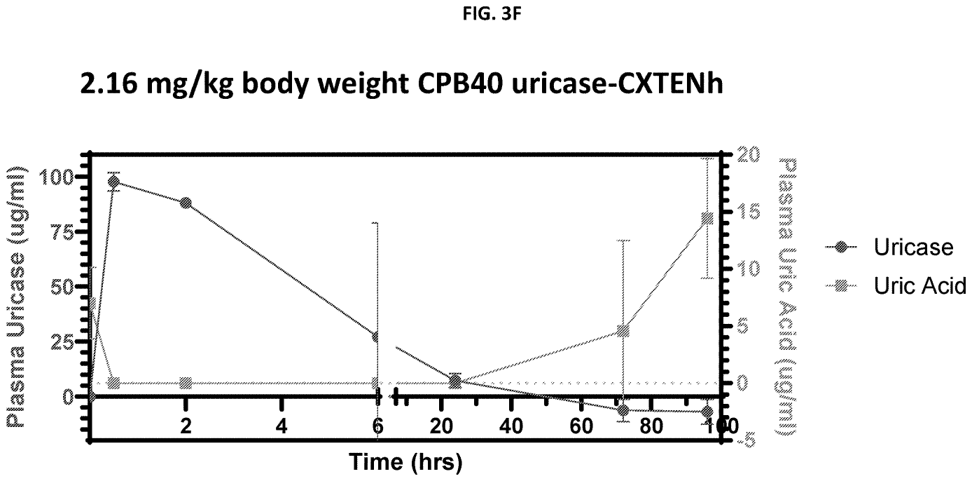

In PK study 1, female Wistar rats in groups of 3 rats each were either left untreated, or treated via intravenous administration with a single dose of 1 mg/kg body weight pegloticase, 1.51 mg/kg body weight CPB40 uricase-CPAS10h, 1.99 mg/kg body weight CPB40 uricase-CPAS20h, 2.47 mg/kg body weight CPB40 uricase-CPAS30h, or 2.16 mg/kg body weight CPB40 uricase-CXTENh (Table 5A). Rat blood samples were collected pre-dose and 0.5 h, 2 h, 6 h, 24 h, 72 h, and 96 h post-dose. The plasma concentrations of the endogenous uricase in the untreated control rats, or pegloticase and uricase fusion proteins in the treated rats, as well as the plasma concentrations of uric acid, were determined, as described in the “Methods.” The results are presented in FIGS. 3A-3F, where the aforementioned endogenous uricase or exogenously administered pegloticase and uricase fusion proteins in the rat plasma are generally referred to as “plasma uricase.”

FIG. 3A shows the plasma concentrations of the endogenous uricase as well as uric acid in the untreated control rats at the corresponding pre-dose timepoint (at time 0) and at the various corresponding post-dose timepoints up to 96 h. The plasma concentration of the endogenous uricase at time 0 was adjusted to 0 μg/mL by subtracting the average pre-dose endogenous plasma uricase concentration as described in the “Methods.” At the corresponding post-dose timepoints, the likewise adjusted plasma concentrations of the endogenous uricase fluctuated below or above 0 μg/mL, as noted in the “Methods” section. The plasma concentrations of uric acid in the untreated control rats also fluctuated and were greater than 0 μg/mL at all timepoints.

FIG. 3B shows the plasma concentrations of pegloticase and uric acid pre-dose (at time 0) and at various timepoints up to 96 h post-dose in the pegloticase-treated rats. The plasma concentrations of pegloticase plateaued between 0.5 h and 6 h post-dose and stayed above detectable levels through at least 96 h post-dose, while the plasma concentrations of uric acid were reduced to and maintained at 0 μg/ml at all timepoints tested post-dose, i.e., from 0.5 h through 96 h post-dose.

FIG. 3C shows the plasma concentrations of CPB40 uricase-CPAS10 h and uric acid pre-dose (at time 0) and at various timepoints up to 96 h post-dose in the CPB40 uricase-CPAS10 h-treated rats. The plasma concentration of CPB40 uricase-CPAS10 h peaked by 0.5 h post-dose and then fell below the detection limit by 24 h post-dose. The plasma concentration of uric acid was reduced to and maintained at 0 μg/ml from 0.5 h through 24 h post-dose, which then rebounded to above 0 μg/ml between 24 h and 72 h post-dose.

FIG. 3D shows the plasma concentrations of CPB40 uricase-CPAS20 h and uric acid pre-dose (at time 0) and at various timepoints up to 96 h post-dose in the CPB40 uricase-CPAS20 h-treated rats. The plasma concentration of CPB40 uricase-CPAS20 h peaked by 0.5 h post-dose, stayed at high levels through 24 h post-dose, and then dropped below the detection limit by 72 h post-dose. The plasma concentration of uric acid was reduced to and maintained at 0 μg/ml from 0.5 h through 24 h post-dose, which then rebounded to above 0 μg/ml between 24 h and 72 h post-dose, similar to the plasma uric acid profile in the CPB40 uricase-CPAS10 h-treated rats.

FIG. 3E shows the plasma concentrations of CPB40 uricase-CPAS30 h and uric acid pre-dose (at time 0) and at various timepoints up to 96 h post-dose in the CPB40 uricase-CPAS30 h-treated rats. The changes in the plasma concentrations of CPB40 uricase-CPAS30 h and uric acid post-dose were similar to those in the CPB40 uricase-CPAS20 h-treated rats. Namely, the plasma concentration of CPB40 uricase-CPAS30 h peaked by 0.5 h post-dose, stayed at high or detectable levels through 24 h post-dose, and then dropped below the detection limit by 72 h post-dose. The plasma concentration of uric acid was reduced to and maintained at 0 μg/ml from 0.5 h through 24 h post-dose, which then rebounded to above 0 μg/ml between 24 h and 72 h post-dose.

FIG. 3F shows the plasma concentrations of CPB40 uricase-CXTENh and uric acid pre-dose (at time 0) and at various timepoints up to 96 h post-dose in the CPB40 uricase-CXTENh-treated rats. The plasma concentration of CPB40 uricase-CXTENh peaked by 0.5 h post-dose, followed by a continuous decline to below the detection limit at 72 h post-dose. The rate of decline was faster than that seen in the CPB40 uricase-CPAS20 h-treated rats (see FIG. 3D). The plasma concentration of uric acid was reduced to and maintained at 0 μg/ml from 0.5 h through 24 h post-dose, which then rebounded to above 0 g/ml between 24 h and 72 h post-dose, similar to the plasma uric acid profile in the CPB40 uricase-CPAS20 h-treated rats.

2.2. PK Study 2 and Efficacy Analyses of Plasma Uric Acid Concentrations in Rat Blood Samples from the Study

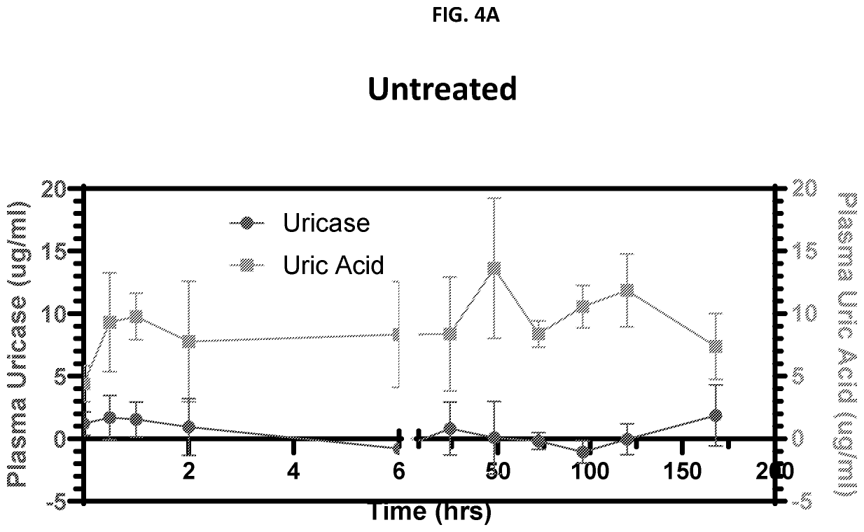

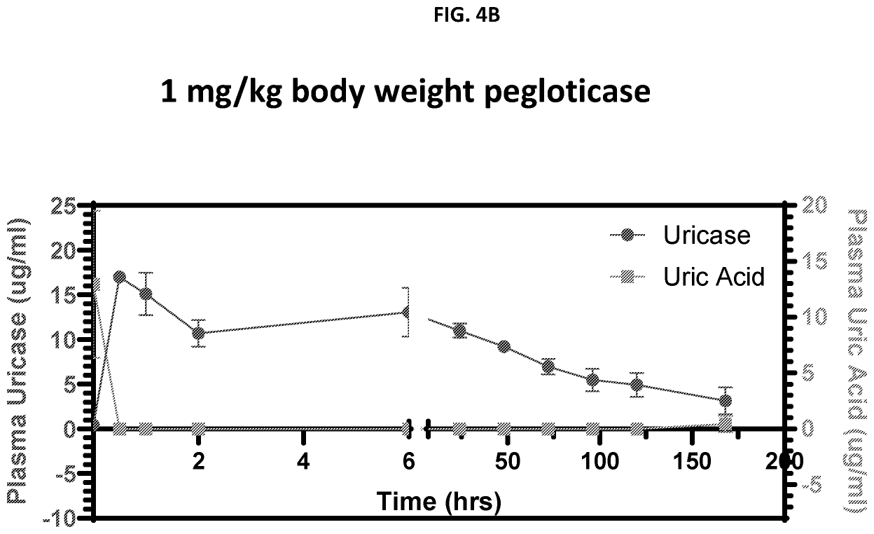

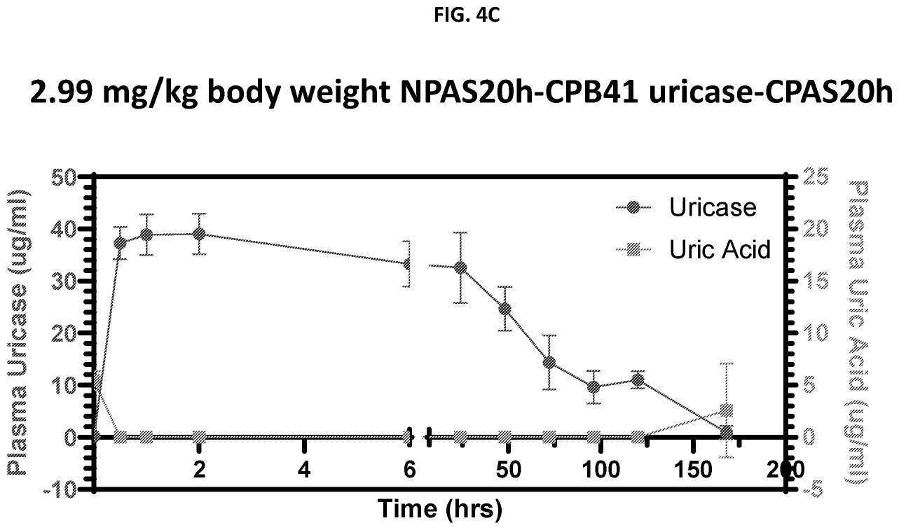

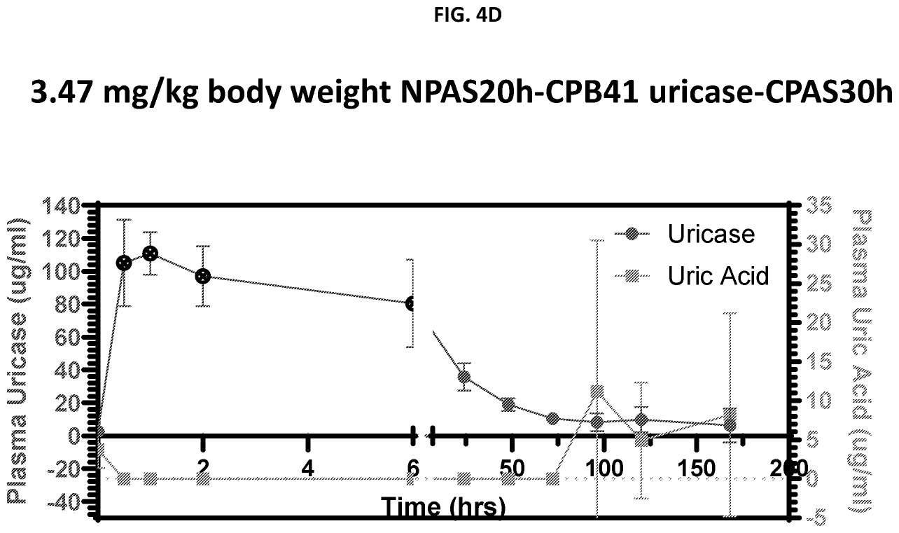

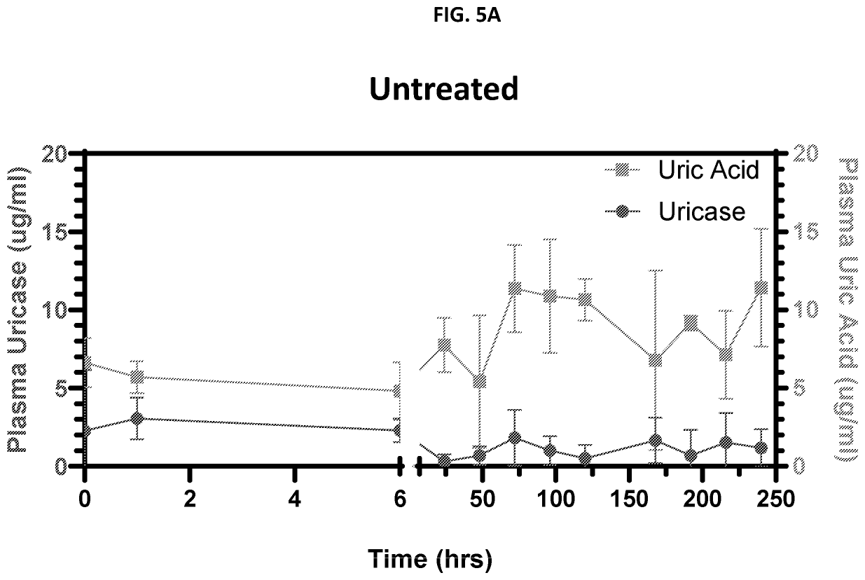

In PK study 2, female Wistar rats in groups of 3 rats each were either left untreated, or treated via intravenous administration with a single dose of 1 mg/kg body weight pegloticase, 2.99 mg/kg body weight NPAS20 h-CPB41 uricase-CPAS20h, or 3.47 mg/kg body weight NPAS20 h-CPB41 uricase-CPAS30h (Table 5B). Rat blood samples were collected pre-dose and 0.5 h, 1 h, 2 h, 6 h, 24 h, 48 h, 72 h, 96 h, 120 h, and 168 h post-dose. The plasma concentrations of the endogenous uricase in the untreated control rats, or pegloticase and uricase fusion proteins in the treated rats, as well as the plasma concentrations of uric acid, were determined, as described in the “Methods.” The results are presented in FIGS. 4A-4D, where the aforementioned endogenous uricase or exogenously administered pegloticase and uricase fusion proteins in the rat plasma are generally referred to as “plasma uricase.”

FIG. 4A shows the plasma concentrations of the endogenous uricase as well as uric acid in the untreated control rats at the corresponding pre-dose timepoint (at time 0) and at the various corresponding post-dose timepoints up to 168 h. The plasma concentration of the endogenous uricase at time 0 was adjusted to near 0 μg/mL by subtracting the average pre-dose endogenous plasma uricase concentration as described in the “Methods.” At the corresponding post-dose timepoints, the likewise adjusted plasma concentrations of the endogenous uricase fluctuated relative to the baseline concentration at time 0. The plasma concentrations of uric acid in the untreated control rats also fluctuated and were greater than 0 μg/mL at all timepoints. These results were similar to those observed in the untreated control rats of PK study 1 presented in FIG. 3A.

FIG. 4B shows the plasma concentrations of pegloticase and uric acid pre-dose (at time 0) and at various timepoints up to 168 h post-dose in the pegloticase-treated rats. The plasma concentrations of pegloticase peaked at 0.5 h post-dose and stayed above detectable levels through at least 168 h post-dose. The plasma concentrations of uric acid were reduced to and maintained at 0 μg/ml through 168 h post-dose in two of the three pegloticase-treated rats, while the plasma uric acid concentration in the third pegloticase-treated rat rebounded to a low level above 0 μg/ml only at the 168 h timepoint, but not at the 120 h timepoint.

FIG. 4C shows the plasma concentrations of NPAS20 h-CPB41 uricase-CPAS20 h and uric acid pre-dose (at time 0) and at various timepoints up to 168 h post-dose in the NPAS20 h-CPB41 uricase-CPAS20 h-treated rats. The changes in the plasma concentrations of NPAS20 h-CPB41 uricase-CPAS20 h and uric acid post-dose were similar to those in the pegloticase-treated rats. Namely, the plasma concentrations of NPAS20 h-CPB41 uricase-CPAS20 h plateaued between 0.5 h and 2 h post-dose and stayed above detectable levels through at least 168 h post-dose. The plasma concentrations of uric acid were reduced to and maintained at 0 μg/ml through 168 h post-dose in two of the three NPAS20 h-CPB41 uricase-CPAS20 h-treated rats, while the plasma uric acid concentration in the third NPAS20 h-CPB41 uricase-CPAS20 h-treated rat rebounded to a low level above 0 μg/ml only at the 168 h timepoint, but not at the 120 h timepoint.

FIG. 4D shows the plasma concentrations of NPAS20 h-CPB41 uricase-CPAS30 h and uric acid pre-dose (at time 0) and at various timepoints up to 168 h post-dose in the NPAS20 h-CPB41 uricase-CPAS30 h-treated rats. The plasma concentration of NPAS20h-CPB41 uricase-CPAS30 h peaked at about 1 h post-dose, stayed at high levels till 6 h post-dose, and then fell to a low steady state level near the detection limit at 72 h and later timepoints post-dose. The plasma concentrations of uric acid were reduced to and maintained at 0 μg/ml through 72 h post-dose, which then rebounded to above 0 g/ml at 96 h and later timepoints post-dose.

2.3. PK Study 3 and Efficacy Analyses of Plasma Uric Acid Concentrations in Rat Blood Samples from the Study

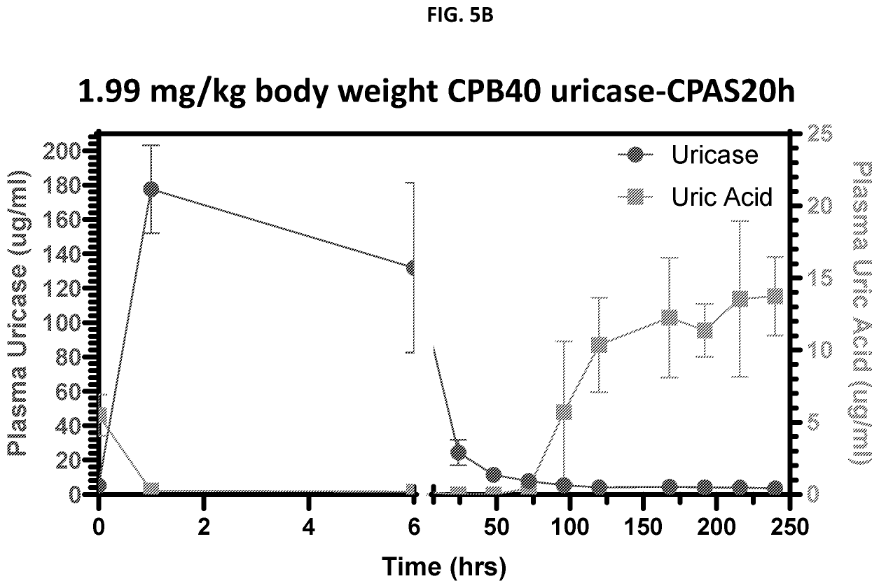

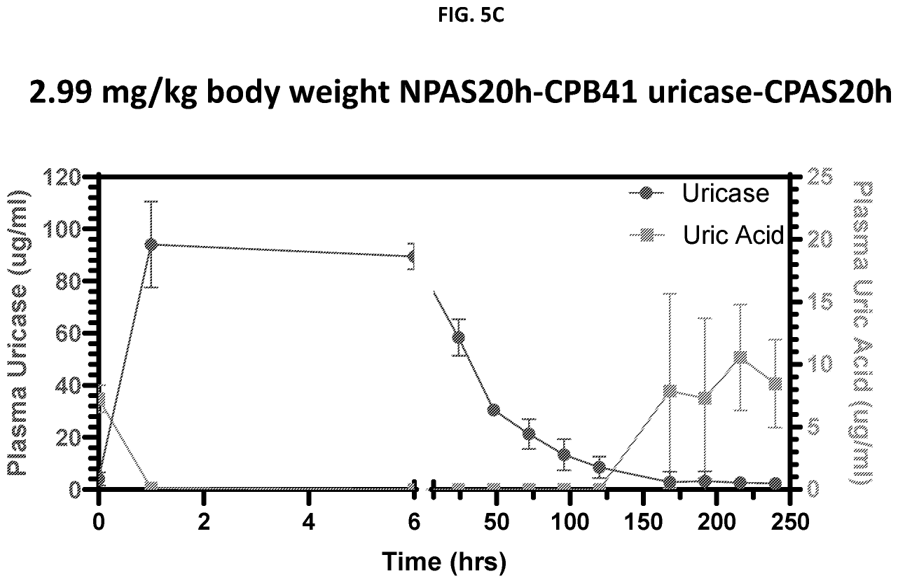

In PK study 3, uricase fusion proteins CPB40 uricase-CPAS20 h studied in PK study 1 and NPAS20 h-CPB41 uricase-CPAS20 h studied in PK study 2 were compared side by side. Specifically, female Wistar rats in groups of 3 rats each were either left untreated, or treated via intravenous administration with a single dose of 1.99 mg/kg body weight CPB40 uricase-CPAS20 h or 2.99 mg/kg body weight NPAS20 h-CPB41 uricase-CPAS20h (Table 5C). Rat blood samples were collected pre-dose and 1 h, 6 h, 24 h, 48 h, 72 h, 96 h, 120 h, 168 h, 192 h, 216 h, and 240 h post-dose. The plasma concentrations of the endogenous uricase in the untreated control rats, or the uricase fusion proteins (i.e., CPB40 uricase-CPAS20 h and NPAS20 h-CPB41 uricase-CPAS20h) in the treated rats, as well as the plasma concentrations of uric acid, were determined, as described in the “Methods.” The results are presented in FIGS. 5A-5C, where the aforementioned endogenous uricase or exogenously administered uricase fusion proteins in the rat plasma are generally referred to as “plasma uricase.”

FIG. 5A shows the plasma concentrations of the endogenous uricase as well as uric acid in the untreated control rats at the corresponding pre-dose timepoint (at time 0) and at the various corresponding post-dose timepoints up to 240 h. The plasma concentration of the endogenous uricase at time 0 was adjusted to near 0 μg/mL by subtracting the average pre-dose endogenous plasma uricase concentration as described in the “Methods.” At the corresponding post-dose timepoints, the likewise adjusted plasma concentrations of the endogenous uricase fluctuated relative to the baseline concentration at time 0. The plasma concentrations of uric acid in the untreated control rats also fluctuated and were greater than 0 μg/mL at all timepoints. These results were similar to those observed in the untreated control rats of PK studies 1 and 2 presented in FIGS. 3A and 4A, respectively.

FIG. 5B shows the plasma concentrations of CPB40 uricase-CPAS20 h and uric acid pre-dose (at time 0) and at various timepoints up to 240 h post-dose in the CPB40 uricase-CPAS20 h-treated rats. The plasma concentration of CPB40 uricase-CPAS20 h peaked at 1 h post-dose (the earliest post-dose timepoint tested in the study), stayed at high levels through 24 h post-dose, and then dropped below the detection limit between 72 h and 96 h post-dose. The plasma concentration of uric acid was reduced to and maintained at 0 μg/ml from 1 h through 72 h post-dose, which then rebounded to above 0 μg/ml between 72 h and 96 h post-dose. The performance of CPB40 uricase-CPAS20 h at 72 h post-dose appeared improved in this study over its performance at the same timepoint (i.e., 72 h post-dose) in PK study 1 (see FIG. 3D), likely due to the higher quality of the uricase fusion protein produced for this PK study (i.e., PK study 3).

FIG. 5C shows the plasma concentrations of NPAS20 h-CPB41 uricase-CPAS20 h and uric acid pre-dose (at time 0) and at various timepoints up to up to 240 h post-dose in the NPAS20 h-CPB41 uricase-CPAS20 h-treated rats. The plasma concentrations of NPAS20 h-CPB41 uricase-CPAS20 h plateaued between 1 h (the earliest post-dose timepoint tested in the study) and 6 h post-dose and stayed above detectable levels through at least 168 h post-dose. The plasma concentrations of uric acid were reduced to and maintained at 0 μg/ml through 120 h post-dose in all three NPAS20 h-CPB41 uricase-CPAS20 h-treated rats. At 168 h post-dose, while the uric acid plasma concentration remained at 0 μg/ml in one of the three treated rats, it rebounded to a low level above 0 μg/ml in two other treated rats in the group. Thus, the PK and efficacy results for NPAS20 h-CPB41 uricase-CPAS20 h in this study were overall similar to those seen in PK study 2 (see FIG. 4C).

In summary, the results from the three PK and efficacy studies above indicate that plasma uric acid concentrations in the untreated control rats varied from rat to rat and at different timepoints of the studies. As such, it was uninformative to interpret the precise plasma uric acid concentration values following treatment with the uricase fusion proteins or pegloticase. However, the study results demonstrate a binary correlation between the plasma uric acid concentration at 0 μg/ml vs. greater than 0 μg/ml and the presence vs. clearance of the circulating exogenous uricase enzyme (i.e., the uricase fusion proteins or pegloticase). Specifically, the plasma uric acid concentration being at 0 μg/ml correlated with, or was indicative of, detectable levels of the circulating exogenous uricase enzyme, while the plasma uric acid concentration rebounding to above 0 μg/ml correlated with, or was indicative of, elimination of the exogenously administered uricase enzyme.

Aliquots of the purified soluble tetrameric uricase fusion protein samples for use in PK studies 2 and 3 were subjected to further characterization in vitro, including uricase enzymatic activity assays, differential scanning fluorimetry (DSF) to determine the proteins' melting temperatures (Tₘ), and dynamic light scattering (DLS) to determine the proteins' hydrodynamic radii and aggregation status, as described below.

3. Uricase Enzymatic Activity of the Uricase Fusion Proteins in Comparison with Pegloticase

Aliquots of the two purified soluble tetrameric uricase fusion protein samples respectively containing NPAS20 h-CPB41 uricase-CPAS20 h and NPAS20 h-CPB41 uricase-CPAS30 h for use in PK study 2, as well as an aliquot of pegloticase, were subjected to the uricase enzymatic activity assays at the protein concentration of 72.8 nM as described in the “Methods,” with the results shown in Table 7A. The uricase enzymatic activity of each sample was measured three times. The average of the three measurements and standard deviation (SD) for each sample were calculated and shown in Table 7A.

TABLE 7A
Uricase enzymatic activity of NPAS20h-CPB41 uricase-CPAS20h,
NPAS20h-CPB41 uricase-CPAS30h, and pegloticase at 72.8 nM

| | Uricase enzymatic activity | | | | |
|---|---|---|---|---|---|
| | (μmol uric acid)/min/mg enzyme) | | | | |
| | Measurement | Measurement | Measurement | | |
| Sample | 1 | 2 | 3 | Average | SD |
| NPAS20h- | −286.4 | −291.4 | −285.4 | −287.7 | 2.6 |
| CPB41 | | | | | |
| uricase- | | | | | |
| CPAS20h | | | | | |
| NPAS20h- | −294.4 | −302.6 | −300.4 | −299.1 | 3.5 |
| CPB41 | | | | | |
| uricase- | | | | | |
| CPAS30h | | | | | |
| Pegloticase | −315 | −331.8 | −336.6 | −327.8 | 9.3 |

The data of Table 7A show that each of the uricase fusion proteins NPAS20h-CPB41 uricase-CPAS20 h and NPAS20 h-CPB41 uricase-CPAS30 h tested in PK study 2 had more than 80% of the activity of pegloticase in vitro.

Aliquots of the two purified soluble tetrameric uricase fusion protein samples respectively containing CPB40 uricase-CPAS20 h and NPAS20 h-CPB41 uricase-CPAS20 h for use in PK study 3, as well as an aliquot of pegloticase, were likewise subjected to the uricase enzymatic activity assays at the protein concentration of 72.8 nM as described in the “Methods,” with the results shown in Table 7B. The uricase enzymatic activity of each sample was measured three times. The average of the three measurements and standard deviation (SD) for each sample were calculated and shown in Table 7B.

TABLE 7B
Uricase enzymatic activity of CPB40 uricase-CPAS20h,NPAS20h-CPB41
uricase-CPAS20h, and pegloticase at 72.8 nM

| | Uricase enzymatic activity | | | | |
|---|---|---|---|---|---|
| | (μmol uric acid)/min/mg enzyme) | | | | |
| | Measurement | Measurement | Measurement | | |
| Sample | 1 | 2 | 3 | Average | SD |
| CPB40 | −187.4 | −182.6 | −194.6 | −188.2 | 6.0 |
| uricase- | | | | | |
| CPAS20h | | | | | |
| NPAS20h- | −178.8 | −208.0 | −205.4 | −197.4 | 16.2 |
| CPB41 | | | | | |
| uricase- | | | | | |
| CPAS20h | | | | | |
| Pegloticase | −234.0 | −222.4 | −221.8 | −226.1 | 6.9 |

The data of Table 7B show that each of the uricase fusion proteins CPB40 uricase-CPAS20 h and NPAS20 h-CPB41 uricase-CPAS20 h tested in PK study 3 also had more than 80% of the activity of pegloticase in vitro.

### 4. Melting Temperatures (Tₘ) of the Uricase Fusion Proteins

Aliquots of the two purified soluble tetrameric uricase fusion protein samples respectively containing NPAS20 h-CPB41 uricase-CPAS20 h and NPAS20 h-CPB41 uricase-CPAS30 h for use in PK study 2 were subjected to DSF to determine the melting temperatures of the uricase fusion proteins as described in the “Methods,” with the results shown in Table 8A. The melting temperature (Tₘ) of each sample was measured twice. The average of the two measurements and standard deviation (SD) for each sample were calculated and shown in Table 8A.

TABLE 8A
Melting temperatures of NPAS20h-CPB41 uricase-CPAS20h
and NPAS20h-CPB41 uricase-CPAS30h measured by DSF

| | Melting temperature (Tₘ, ° C.) | | | |
|---|---|---|---|---|
| | Measurement | Measurement | | |
| Sample | 1 | 2 | Average | SD |
| NPAS20h- | 63.5 | 63.5 | 63.5 | 0.0 |
| CPB41 | | | | |
| uricase- | | | | |
| CPAS20h | | | | |
| NPAS20h- | 63.5 | 63.5 | 63.5 | 0.0 |
| CPB41 | | | | |
| uricase- | | | | |
| CPAS30h | | | | |

The data of Table 8A show that the uricase fusion proteins NPAS20 h-CPB41 uricase-CPAS20 h and NPAS20 h-CPB41 uricase-CPAS30 h tested in PK study 2 had the same Tₘ of 63.5° C., higher than the historic Tₘ of 61° C. for PEGylated CPB40 uricase determined in prior studies.

Aliquots of the two purified soluble tetrameric uricase fusion protein samples respectively containing CPB40 uricase-CPAS20 h and NPAS20 h-CPB41 uricase-CPAS20 h for use in PK study 3 were likewise subjected to DSF to determine the melting temperatures of the uricase fusion proteins as described in the “Methods,” with the results shown in Table 8B. The melting temperature (Tₘ) of each sample was measured twice. The average of the two measurements and standard deviation (SD) for each sample were calculated and shown in Table 8B.

TABLE 8B
Melting temperatures of CPB40 uricase-CPAS20h and
NPAS20h-CPB41uricase-CPAS20h measured by DSF

| | Melting temperature (Tₘ, ° C.) | | | |
|---|---|---|---|---|
| | Measurement | Measurement | | |
| Sample | 1 | 2 | Average | SD |
| CPB40 | 63.00 | 63.00 | 63.00 | 0.00 |
| uricase- | | | | |
| CPAS20h | | | | |
| NPAS20h- | 63.50 | 63.50 | 63.50 | 0.00 |
| CPB41 | | | | |
| uricase- | | | | |
| CPAS20h | | | | |

The data of Table 8B show that the uricase fusion proteins CPB40 uricase-CPAS20 h and NPAS20 h-CPB41 uricase-CPAS20 h tested in PK study 3 had the melting temperatures of 63.00° C. and 63.50° C., respectively, both of which were higher than the historic Tₘ of 61° C. for PEGylated CPB40 uricase.

### 5. DLS Analyses of the Uricase Fusion Proteins

Aliquots of the two purified soluble tetrameric uricase fusion protein samples respectively containing NPAS20 h-CPB41 uricase-CPAS20 h and NPAS20 h-CPB41 uricase-CPAS30 h for use in PK study 2 were subjected to DLS to determine the hydrodynamic radii, polydispersity, and percent mass in various mass ranges of the uricase fusion proteins as described in the “Methods,” with the results shown in Table 9A.

TABLE 9A
DLS analysis data of NPAS20h-CPB41 uricase-CPAS20h and NPAS20h-CPB41 uricase-CPAS30h

| | | | % Mass (M) | | |
|---|---|---|---|---|---|
| | | | Mass Range 1 | Mass Range 2 | Mass Range 3 |
| | | | (0.5-10 nm | (10-100 nm | (100-1000 nm |
| | Radius | Polydispersity | hydrodynamic | hydrodynamic | hydrodynamic |
| Sample | (nm) | (%) | radius) | radius) | radius) |
| NPAS20h- | 17.65 | 7.3 | — | 100 | — |
| CPB41 | | | | | |
| uricase- | | | | | |
| CPAS20h | | | | | |
| NPAS20h- | 17.2 | 14.3 | 52.45 | 46.85 | — |
| CPB41 | | | | | |
| uricase- | | | | | |
| CPAS30h | | | | | |

The DLS data of Table 9A show that NPAS20 h-CPB41 uricase-CPAS20 h had a hydrodynamic radius of 17.65 nm (with 7.3% polydispersity), and that NPAS20 h-CPB41 uricase-CPAS30 h had a hydrodynamic radius of 17.2 nm (with 14.3% polydispersity).

Additionally, NPAS20 h-CPB41 uricase-CPAS30 h seemed to have two distinct species: one at the expected hydrodynamic radius (~17 nm, 46.85% mass in mass range 2) and another that was smaller than the tetramer species (52.45% mass in mass range 1). Those data suggest that the NPAS20 h-CPB41 uricase-CPAS30 h sample might contain a population of stabilized monomeric protein distinct from the tetrameric population, and that the presence of the monomeric protein population might account for the shorter half-life of the NPAS20h-CPB41 uricase-CPAS30 h sample as compared to the NPAS20 h-CPB41 uricase-CPAS20h sample observed in PK study 2 (see and compare FIGS. 4C and 4D).

Aliquots of the two purified soluble tetrameric uricase fusion protein samples respectively containing CPB40 uricase-CPAS20 h and NPAS20 h-CPB41 uricase-CPAS20 h for use in PK study 3 were likewise subjected to DLS to determine the hydrodynamic radii, polydispersity, and percent mass in mass range 2 (corresponding to 10-100 nm in hydrodynamic radius) of the uricase fusion proteins as described in the “Methods,” with the results shown in Table 9B. Each of the DLS parameters for each sample was measured one or two times (indicated as “1” and “2” in Table 9B), with the average of the two measurements of each parameter calculated and shown in Table 9B.

TABLE 9B
DLS analysis data of CPB40 uricase-CPAS20h
and NPAS20h-CPB41 uricase-CPAS20h

| | | | | | | | % Mass (M) in | | |
|---|---|---|---|---|---|---|---|---|---|
| | | | | | | | Mass Range 2 (10-100 nm | | |
| | Radius (nm) | | | Polydispersity (%) | | | hydrodynamic radius) | | |
| Sample | 1 | 2 | Average | 1 | 2 | Average | 1 | 2 | Average |
| CPB40 | 15.3 | 15.2 | 15.25 | 14.3 | 13.2 | 13.75 | 99.6 | 100 | 99.8 |
| uricase- | | | | | | | | | |
| CPAS20h | | | | | | | | | |
| NPAS20h- | 17.9 | — | 17.9 | 6.3 | — | 6.3 | 100 | — | 100 |
| CPB41 | | | | | | | | | |
| uricase- | | | | | | | | | |
| CPAS20h | | | | | | | | | |

The DLS data of Table 9B show that both of the CPB40 uricase-CPAS20 h and NPAS20 h-CPB41 uricase-CPAS20 h samples had >99% mass in mass range 2 corresponding to 10-100 nm in hydrodynamic radius, indicating <1% aggregation. Additionally, the finding that the hydrodynamic radius of NPAS20 h-CPB41 uricase-CPAS20 h was greater than that of CPB40 uricase-CPAS20 h indicates that the hydrodynamic radius increased with the increasing length of the PAS domain in the uricase fusion proteins.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A uricase conjugate comprising a first domain and a second domain, wherein the first domain comprises a uricase polypeptide, or an amino acid variant thereof, and the second domain is a first random coil polypeptide domain comprising at least about 100 amino acids.

2. The uricase conjugate of claim 1, wherein the uricase conjugate is a fusion protein of the first domain and the second domain.

3. The uricase conjugate of claim 2, wherein the first domain is C-terminal to the second domain.

4. The uricase conjugate of claim 2, wherein the first domain is N-terminal to the second domain.

5. The uricase conjugate of any one of claims 2-4, wherein an amino acid linker is present between the first domain and the second domain.

6. The uricase conjugate of claim 5, wherein the amino acid linker is from about two amino acids long to about 5 amino acids long.

7. The uricase conjugate of claim 6, wherein the amino acid linker is two amino acids long.

8. The uricase conjugate of claim 7, wherein the amino acid linker is Gly-Ser.

9. The uricase conjugate of any one of claims 2-8, further comprising a third domain, wherein the third domain comprises a second random coil polypeptide domain comprising at least about 100 amino acids.

10. The uricase conjugate of claim 9, wherein the first domain is N-terminal to the second domain and C-terminal to the third domain.

11. The uricase conjugate of claim 10, wherein an amino acid linker is present between the first domain and the second domain.

12. The uricase conjugate of claim 10 or 11, wherein an amino acid linker is present between the second domain and the third domain.

13. The uricase conjugate of claim 11 or 12, wherein the amino acid linker is from about two amino acids long to about 5 amino acids long.

14. The uricase conjugate of claim 13, wherein the amino acid linker is two amino acids long.

15. The uricase conjugate of claim 14, wherein the amino acid linker is Gly-Ser.

16. The uricase conjugate of any one of claims 1-15, wherein the second domain comprises a Pro-Ala-Ser (PAS) polypeptide.

17. The uricase conjugate of any one of claims 9-15, wherein the third domain comprises a Pro-Ala-Ser (PAS) polypeptide.

18. The uricase conjugate of claim 16 or 17, wherein the PAS polypeptide has the amino acid sequence set forth in SEQ ID NO: 60.

19. The uricase conjugate of claim 16 or 17, wherein the PAS polypeptide has the amino acid sequence set forth in SEQ ID NO: 61.

20. The uricase conjugate of claim 16 or 17, wherein the PAS polypeptide has the amino acid sequence set forth in SEQ ID NO: 62.

21. The uricase conjugate of claim 16 or 17, wherein the PAS polypeptide comprises an amino acid sequence set forth in SEQ ID NO: 48, SEQ ID NO: 50, SEQ ID NO: 52, SEQ ID NO: 54, SEQ ID NO: 56 or SEQ ID NO: 58.

22. The uricase conjugate of claim 21, wherein the PAS polypeptide comprises the amino acid sequence of SEQ ID NO: 48.

23. The uricase conjugate of claim 22, wherein the amino acid sequence of SEQ ID NO: 48 is encoded by a nucleotide sequence selected from SEQ ID NOs: 81-111.

24. The uricase conjugate of any one of claims 1-15, wherein the second domain comprises an extended recombinant (XTEN) polypeptide.

25. The uricase conjugate of any one of claims 9-15, wherein the third domain comprises an extended recombinant (XTEN) polypeptide.

26. The uricase conjugate of claim 24 or 25, wherein the XTEN polypeptide has the amino acid sequence set forth in SEQ ID NO:74.

27. The uricase conjugate of any one of claims 1-26, wherein the first random coil polypeptide comprises from about 100 amino acids to about 800 amino acids.

28. The uricase conjugate of any one of claims 9-27, wherein the second random coil polypeptide domain comprises from about 100 amino acids to about 800 amino acids.

29. The uricase conjugate of any one of claims 1-28, wherein the first random coil polypeptide comprises from about 100 amino acids to about 700 amino acids.

30. The uricase conjugate of any one of claims 9-29, wherein the second random coil polypeptide domain comprises from about 100 amino acids to about 700 amino acids.

31. The uricase conjugate of any one of claims 1-30, wherein the first random coil polypeptide comprises from about 100 amino acids to about 600 amino acids.

32. The uricase conjugate of any one of claims 9-31, wherein the second random coil polypeptide domain comprises from about 100 amino acids to about 600 amino acids.

33. The uricase conjugate of any one of claims 1-32, wherein the first random coil polypeptide comprises from about 100 amino acids to about 500 amino acids.

34. The uricase conjugate of any one of claims 9-33, wherein the second random coil polypeptide domain comprises from about 100 amino acids to about 500 amino acids.

35. The uricase conjugate of any one of claims 1-34, wherein the first random coil polypeptide comprises from about 100 amino acids to about 400 amino acids.

36. The uricase conjugate of any one of claims 9-35, wherein the second random coil polypeptide domain comprises from about 100 amino acids to about 400 amino acids.

37. The uricase conjugate of any one of claims 1-36, wherein the first random coil polypeptide comprises from about 100 amino acids to about 300 amino acids.

38. The uricase conjugate of any one of claims 9-37, wherein the second random coil polypeptide domain comprises from about 100 amino acids to about 300 amino acids.

39. The uricase conjugate of claim 1, comprising the amino acid sequence set forth in SEQ ID NO:63.

40. The uricase conjugate of claim 1, comprising the amino acid sequence set forth in SEQ ID NO:64.

41. The uricase conjugate of claim 1, comprising the amino acid sequence set forth in SEQ ID NO:65.

42. The uricase conjugate of claim 1, comprising the amino acid sequence set forth in SEQ ID NO:66.

43. The uricase conjugate of claim 1, comprising the amino acid sequence set forth in SEQ ID NO:67.

44. The uricase conjugate of claim 1, comprising the amino acid sequence set forth in SEQ ID NO:68.

45. The uricase conjugate of claim 1, comprising the amino acid sequence set forth in SEQ ID NO:69.

46. The uricase conjugate of claim 1, comprising the amino acid sequence set forth in SEQ ID NO:70.

47. The uricase conjugate of claim 1, comprising the amino acid sequence set forth in SEQ ID NO:75.

48. The uricase conjugate of claim 1, comprising the amino acid sequence set forth in SEQ ID NO:76.

49. The uricase conjugate of claim 1, comprising the amino acid sequence set forth in SEQ ID NO:77.

50. The uricase conjugate of claim 1, comprising the amino acid sequence set forth in SEQ ID NO:78.

51. The uricase conjugate of claim 1, comprising the amino acid sequence set forth in SEQ ID NO:79.

52. The uricase conjugate of claim 1, comprising the amino acid sequence set forth in SEQ ID NO:80.

53. The uricase conjugate of claim 1, comprising the amino acid sequence set forth in SEQ ID NO:112.

54. The uricase conjugate of claim 1, comprising the amino acid sequence set forth in SEQ ID NO:113.

55. The uricase conjugate of any one of claims 1-38, wherein the uricase domain comprises the amino acid sequence set forth in SEQ ID NO: 1.

56. The uricase conjugate of any one of claims 1-38, wherein the uricase domain comprises the amino acid sequence set forth in SEQ ID NO: 2.

57. The uricase conjugate of any one of claims 1-38, wherein the uricase domain comprises the amino acid sequence set forth in SEQ ID NO: 3.

58. The uricase conjugate of any one of claims 1-38, wherein the uricase domain comprises the amino acid sequence set forth in SEQ ID NO: 4.

59. The uricase conjugate of any one of claims 1-38, wherein the uricase domain comprises the amino acid sequence set forth in SEQ ID NO: 5.

60. The uricase conjugate of any one of claims 1-38, wherein the uricase domain comprises an amino acid sequence selected from SEQ ID NO: 6-39.

61. The uricase conjugate of any one of claims 1-38, wherein the uricase domain comprises an amino acid sequence selected from SEQ ID NO: 40-44.

62. The uricase conjugate of any one of claims 1-38, wherein the uricase domain comprises the amino acid sequence set forth in SEQ ID NO: 40.

63. The uricase conjugate of any one of claims 1-38, wherein the uricase domain comprises the amino acid sequence set forth in SEQ ID NO: 41.

64. The uricase conjugate of any one of claims 1-38, wherein the uricase domain comprises the amino acid sequence set forth in SEQ ID NO: 42.

65. The uricase conjugate of any one of claims 1-38, wherein the uricase domain comprises the amino acid sequence set forth in SEQ ID NO: 43.

66. The uricase conjugate of any one of claims 1-38, wherein the uricase domain comprises the amino acid sequence set forth in SEQ ID NO: 44.

67. The uricase conjugate of any one of claims 1-38, wherein the uricase domain comprises the amino acid sequence set forth in SEQ ID NO: 45.

68. The uricase conjugate of any one of claims 1-38, wherein the uricase domain comprises the amino acid sequence set forth in SEQ ID NO: 46.

69. The uricase conjugate of any one of claims 1-38, wherein the uricase domain comprises the amino acid sequence set forth in SEQ ID NO: 47.

70. The uricase conjugate of any one of claims 1-38, wherein the uricase domain comprises an amino acid variant of a uricase polypeptide.

71. The uricase conjugate of claim 70, wherein the amino acid variant has an amino acid sequence with at least about 75% identity to the uricase polypeptide.

72. The uricase conjugate of claim 70, wherein the amino acid variant has an amino acid sequence with at least about 80% identity to the uricase polypeptide.

73. The uricase conjugate of claim 70, wherein the amino acid variant has an amino acid sequence with at least about 85% identity to the uricase polypeptide.

74. The uricase conjugate of claim 70, wherein the amino acid variant has an amino acid sequence with at least about 90% identity to the uricase polypeptide.

75. The uricase conjugate of claim 70, wherein the amino acid variant has an amino acid sequence with at least about 95% identity to the uricase polypeptide.

76. The uricase conjugate of any one of claims 70-75, wherein the amino acid variant is a variant of the uricase polypeptide having the amino acid sequence set forth in SEQ ID NO: 40.

77. The uricase conjugate of any one of claims 70-75, wherein the amino acid variant is a variant of the uricase polypeptide having the amino acid sequence set forth in SEQ ID NO: 41.

78. The uricase conjugate of claim 70, wherein the amino acid variant comprises from about 10 to about 20 amino acid substitutions in the uricase polypeptide.

79. The uricase conjugate of claim 70, wherein the amino acid variant comprises from about 10 to about 18 amino acid substitutions in the uricase polypeptide.

80. The uricase conjugate of claim 70, wherein the amino acid variant comprises from about 10 to about 16 amino acid substitutions in the uricase polypeptide.

81. The uricase conjugate of claim 70, wherein the amino acid variant comprises from about 10 to about 14 amino acid substitutions in the uricase polypeptide.

82. The uricase conjugate of claim 70, wherein the amino acid variant comprises from about 10 to about 13 amino acid substitutions in the uricase polypeptide.

83. The uricase conjugate of claim 70, wherein the amino acid variant comprises from about 10 to about 12 amino acid substitutions in the uricase polypeptide.

84. The uricase conjugate of claim 70, wherein the amino acid variant comprises from about 11 to about 20 amino acid substitutions in the uricase polypeptide.

85. The uricase conjugate of claim 70, wherein the amino acid variant comprises from about 12 to about 20 amino acid substitutions in the uricase polypeptide.

86. The uricase conjugate of claim 70, wherein the amino acid variant comprises from about 13 to about 20 amino acid substitutions in the uricase polypeptide.

87. The uricase conjugate of claim 70, wherein the amino acid variant comprises from about 14 to about 20 amino acid substitutions in the uricase polypeptide.

88. The uricase conjugate of claim 70, wherein the amino acid variant comprises from about 15 to about 20 amino acid substitutions in the uricase polypeptide.

89. The uricase conjugate of any one of claims 78-88, wherein the amino acid variant is a variant of the uricase polypeptide having the amino acid sequence set forth in SEQ ID NO: 40.

90. The uricase conjugate of any one of claims 78-88, wherein the amino acid variant is a variant of the uricase polypeptide having the amino acid sequence set forth in SEQ ID NO: 41.

91. The uricase conjugate of any one of claims 2-90, wherein the uricase domain does not include an N-terminal methionine residue.

92. The uricase conjugate of any one of claims 2-91, wherein the first random coil polypeptide domain does not include an N-terminal methionine residue.

93. The uricase conjugate of any one of claims 9-92, wherein the second random coil polypeptide domain does not include an N-terminal methionine residue.

94. The uricase conjugate of any one of claims 2-93, further comprising a purification tag at the C-terminus.

95. The uricase conjugate of any one of claims 2-94, further comprising a purification tag at the N-terminus.

96. The uricase conjugate of claim 94 or 95, wherein the purification tag is a polyhistidine tag.

97. The uricase conjugate of any one of claims 1-96, wherein the uricase conjugate is a monomer.

98. The uricase conjugate of any one of claims 1-97, wherein the uricase conjugate is present within a homotetramer.

99. The uricase conjugate of any one of claims 1-96, wherein the uricase conjugate is a homotetramer.

100. A nucleic acid which encodes the uricase conjugate of any one of claims 1-99.

101. A nucleic acid vector comprising the nucleic acid of claim 100.

102. A host cell comprising the nucleic acid vector of claim 101.

103. A pharmaceutical composition comprising the uricase conjugate of any one of claims 1-99.

104. A method of treating hyperuricemia in a subject in need of treatment, comprising administering to the subject an effective amount of the uricase conjugate of any one of claims 1-99, or the pharmaceutical composition of claim 103.

105. The method of claim 104, wherein uric acid levels are reduced in the plasma of the subject.

106. The method of claim 104 or 105, wherein the subject is a gout patient.

107. The method of claim 106, wherein the subject is a refractory gout patient.

108. The method of any one of claims 104-107, wherein the subject has been diagnosed with tumor lysis syndrome.

109. A method of treating gout in a subject in need of treatment, comprising administering to the subject an effective amount of the uricase conjugate of any one of claims 1-99 or the pharmaceutical composition of claim 103.

110. The method of claim 109, wherein the gout is refractory gout.

111. A method of treating tumor lysis syndrome in a subject in need of treatment, comprising administering to the subject an effective amount of the uricase conjugate of any one of claims 1-99 or the pharmaceutical composition of claim 103.

112. The method of any one of claims 104-111, wherein the subject is a human patient.

113. The method of claim 112, wherein the human patient is an adult human patient.

114. The method of any one of claims 104-113, wherein administering comprises parenteral administering.

115. The method of claim 114, wherein the parenteral administering comprises intravenous administering.

116. The method of claim 114, wherein the parenteral administering comprises subcutaneous administering.

117. A method of recombinantly producing the uricase conjugate of any one of claims 1-99, comprising:
(i) culturing a host cell comprising a nucleic acid vector comprising a nucleic acid sequence encoding the uricase conjugate of any one of claims 1-99, wherein the nucleic acid sequence is operatively linked to a heterologous promoter under conditions to allow for expression of the nucleic acid sequence encoding the uricase conjugate and recombinant production of the uricase conjugate by the host cell; and
(ii) isolating the recombinantly produced uricase conjugate.
